SEQUENCE LISTING (73 sequences)

SEQ ID NO: 1 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 2 - protein, 2458 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAVKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

SEQ ID NO: 3 - protein, 2448 aa, Mus sp.
MVLLLFLTCLVFSCLTFSWLKIWGKMTDSKPLTNSKVEANLLSSEESLSASELSGEQLQEHGDHSCLSYRGPRDASQQRNSLPSSCQRPPRNPLSSNDTWPSPELQTNWTAAPGPEVPDANGLSFPARPPSQRTVSPSREDRKQAHIKRQLMTSFILGSLDDNSSDEDPSAGSFQNSSRKSSRASLGTLSQEAALNTSDPESHAPTMRPSMSGLHLVKRGREHKKLDLHRDFTVASPAEFVTRFGGNRVIEKVLIANNGIAAVKCMRSIRRWAYEMFRNERAIRFVVMVTPEDLKANAEYIKMADQYVPVPGGPNNNNYANVELIIDIAKRIPVQAVWAGWGHASENPKLPELLCKHEIAFLGPPSEAMWALGDKIASTIVAQTLQIPTLPWSGSGLTVEWTEDSRHQGKCISVPEDVYEQGCVKDVDEGLQAAEKIGFPLMIKASEGGGGKGIRKAESAEDFPMLFRQVQSEIPGSPIFLMKLAQNARHLEVQVLADQYGNAVSLFGRDCSIQRRHQKIIEEAPATIAAPAVFEFMEQCAVLLAKMVGYVSAGTVEYLYSQDGSFHFLELNPRLQVEHPCTEMIADVNLPAAQLQIAMGVPLHRLKDIRLLYGESPWGVTPIPFETPLSPPIARGHVIAARITSENPDEGFKPSSGTVQELNFRSNKNVWGYFSVAAAGGLHEFADSQFGHCFSWGENREEAISNMVVALKELSIRGDFRTTVEYLVNLLETESFQNNDIDTGWLDHLIAQRVQAEKPDIMLGVVCGALNVADAMFRTCMTEFLHSLERGQVLPADSLLNIVDVELIYGGIKYALKVARQSLTMFVLIMNGCHIEIDAHRLNDGGLLLSYNGSSYTTYMKEEVDSYRITIGNKTCVFEKENDPTVLRSPSAGKLMQYTVEDGDHVEAGSSYAEMEVMKMIMTLNVQESGRVKYIKRPGVILEAGCVVARLELDDPSKVHAAQPFTGELPAQQTLPILGEKLHQVFHGVLENLTNVMSGYCLPEPFFSMKLKDWVQKLMMTLRHPSLPLLELQEIMTSVAGRIPAPVEKAVRRVMAQYASNITSVLCQFPSQQIATILDCHAATLQRKADREVFFMNTQSIVQLVQRYRSGTRGYMKAVVLDLLRKYLNVEHHFQQAHYDKCVINLREQFKPDMTQVLDCIFSHSQVAKKNQLVTMLIDELCGPDPTLSDELTSILCELTQLSRSEHCKVALRARQVLIASHLPSYELRHNQVESIFLSAIDMYGHQFCPENLKKLILSETTIFDVLPTFFYHENKVVCMASLEVYVRRGYIAYELNSLQHRELPDGTCVVEFQFMLPSSHPNRMAVPISVSNPDLLRHSTELFMDSGFSPLCQRMGAMVAFRRFEEFTRNFDEVISCFANVQTDTLLFSKACTSLYSEEDSKSLREEPIHILNVAIQCADHMEDEALVPVFRAFVQSKKHILVDYGLRRITFLVAQEREFPKFFTFRARDEFAEDRIYRHLEPALAFQLELSRMRNFDLTAVPCANHKMHLYLGAAKVKEGLEVTDHRFFIRAIIRHSDLITKEASFEYLQNEGERLLLEAMDELEVAFNNTSVRTDCNHIFLNFVPTVIMDPLKIEESVRDMVMRYGSRLWKLRVLQAEVKINIRQTTSDSAIPIRLFITNESGYYLDISLYREVTDSRSGNIMFHSFGNKQGSLHGMLINTPYVTKDLLQAKRFQAQSLGTTYVYDFPEMFRQALFKLWGSPEKYPKDILTYTELVLDSQGQLVEMNRLPGCNEVGMVAFKMRFKTPEYPEGRDAVVIGNDITFQIGSFGIGEDFLYLRASEMARTEGIPQIYLAANSGARMGLAEEIKQIFQVAWVDPEDPHKGFRYLYLTPQDYTQISSQNSVHCKHIEDEGESRYVIVDVIGKDANLGVENLRGSGMIAGEASLAYEKTVTISMVTCRALGIGAYLVRLGQRVIQVENSHIILTGAGALNKVLGREVYTSNNQLGGVQIMHTNGVSHVTVPDDFEGVCTILEWLSFIPKDNRSPVPITTPSDPIDREIEFTPTKAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQVIRDFNKERLPLMIFANWRGFSGGMKDMYEQMLKFGAYIVDGLRLYEQPILIYIPPCAELRGGSWVVLDSTINPLCIEMYADKESRGGVLEPEGTVEIKFRKKDLVKTIRRIDPVCKKLVGQLGKAQLPDKDRKELEGQLKAREELLLPIYHQVAVQFADLHDTPGHMLEKGIISDVLEWKTARTFFYWRLRRLLLEAQVKQEILRASPELNHEHTQSMLRRWFVETEGAVKAYLWDSNQVVVQWLEQHWSAKDGLRSTIRENINYLKRDSVLKTIQSLVQEHPEVIMDCVAYLSQHLTPAERIQVAQLLSTTESPASS

SEQ ID NO: 4 - protein, 2456 aa, Rattus sp.
MVLLLFLTYLVFSCLTISWLKIWGKMTDSRPLSNSKVDASLLPSKEESFASDQSEEHGDCSCPLTTPDQEELASHGGPVDASQQRNSVPTSHQKPPRNPLSSNDTCSSPELQTNGVAAPGSEVPEANGLPFPARPQTQRTGSPTREDKKQAPIKRQLMTSFILGSLDDNSSDEDPSSNSFQTSSRKGSRDSLGTCSQEAALNTADPESHTPTMRPSMSGLHLVKRGREHKKLDLHRDFTVASPAEFVTRFGGNRVIETVLIANNGIAAVKWMRSIRRWAYEMFRNERAIRFVVMVTPEDLKANAEYYKMADPVLPVPGGPNNNNYANVELIIDIAKRIPVQAVWAGWGHASENPKLPELLCKHGIAFLGPRVRPMLGLGDRLSSTIVAQTLQIPTLPWSGSGLTVEWTEDSQHQGKCISVTEDVYEQGCVRDVDEGLQAAEKVGFPLMIKASEGGGGKGIRQAESAEDFPCFFRQVQSEIPGSPIFLMKLAQNARHLEVQVLADQYGNAVSLFGRDCSIQRRHQKIIEEAPANIAAPAVFEFMEQCAVLLAKTVVYVSAGTVGYLYSQDGSFHFLELNPRLQVEHPCTEMIADVNLPAAQLQIAMGVPLHRLKDIRLLYGESPWGVTPVSFETPLSPPIARGHVIAARITSENPDEAFKPSSGTVQELNFRSNKNVWGYFSVAAAGGLHEFPISQFGHCFSWGENQEEAISNMVVALKELSIRGDFRTTVEYLVNLLETESLQNNDIDTGWLDHLIAQRVQAEKPDIMLGVVFGALNVADAMFRTCITEFLHSLERGQVLPADSLLNIVDVELIYGGIKYVLKVARQSLTMFVLIMNGCHIEIDAHRPNDGGLLLSYNGSSYTTYMKEEVDSYRITIGNKTCVFEKENDPTVLRSPSAGKLMQYTVEDGQHVEVGSSYAEMEVMKMIMTLNVQESGRVNYIKRPGAVLEAGCVVAKLELDDPSKVHAAQPFTGELPAQQTLPILGERLHQVFHSVLENLTNVMNGYCLPEPFFSMKLKDWVEKPMMTLRHPSLPLLELQEIMTSVADRIPVPVEKAVRRVFAQDASNITSVLCQFPSQQIATILDCHAATLQRKVDREAFFMNTQSIVQLIQRYRSGTRGIMKAVVLDLLRRYLNVEHHFQQAHYDKCVINLREQFKADMTRVLDCIFSHSQVAKKNQLVTMLIDELCGPDPTLSEELTSILKELTQLSRSEHCKVALRARQVLIASHLPSYELRHNQVESSSCQPLTCNGHQFCPENLKKLILSETTIFDVLPTFFYHANKVVCMASLEVYVRRGYIAYELNSLQHRELPDGTCVVEFQFMLPSSHPNRMAMPINVSDPDLLRHSKELFMDSGFSPLCHQRMGAMVAFRRFEEFTRNFDEVISCFANVPTDTPLFSKACTSLYSEEDSKSLQEEPIHILNVAIQCADHMEDERLVPVFRAFVQSKKHILVDYGLRRITFLIAQEKEFPKFFTFRARDEFAEDRIYRHLEPGLAFQLELSRMRNFDLTAVPCANHKMHLYLGAAKVKEGLEVTDHRFFIRAIIRHSDLITKEASFEYLQNEGERLLLEAMDELEVAFNNTSVRTDCNHIFLNFVAHVIMDPLKIEESVRAMVMRYGSRLWKLRVLQAQVKINIRQTTSDCAVPIRLFITNESGYYLDISLYKEVTDSRSGNIMFHSFGNKQGSLHGMLINTPYVTKDLLQAKRFQAQSLGTTYVYDFPEMFRQALFKLWGSPEKYGPDILTYTELVLDSQGQLVEMNRLPGCNEVGMVVFKMRFKTPEYPEGRDTIVIGNDITFQIGSFGIGEDFLYLRASEMARTEGIPQIYLAANSGAVLGLSEEIKQIFQVAWVDPEDPYKGFRYLYLYLTPQDYTQISSQNSVHCKHIEDEGESGIIVDVIGKDSSLGVENLRGSGMIAGEASLAYEKNVTISMVDCRAIGIGAYLVRLGQRVIQVENSHIILTGAGALNKVLGREVYTSNNQLGGVQIMHTNGVSHVTVPDDFEGVCTILEWLSYIPKDNQSPVPIITPSDPIDREIEFTPTKAPYDPRWLLAGRPHPTLKGTWQSGFFDHGSFKEIMAPWDQTVVTGRARLGGIPVGVIAVETRSVEVAVPAHPANLDSEAKIIQQAGQVWFPDSAFKTAQVIRDFNQEHLLLMIFANWRGFSGGMKDMSEQMLKFGAYIVDSLRLSKQPVLIYIPPGAELRGGSWVVLDSSINPLCIEMYADKESRGGVLEPEGTVEIKFRKKDLVKTIRRIDPVCKKLLEPAGDTQLPDKDRKELESQLKAREDLLLPIYHQVAVQFADLHDTPGHMLKKGIISDVLEWKTTRTYFYWRLRRLLLEAQVKQEILRASPELSHEHTQSMLRRWFVETEGAVKAYLWDSNQVVVQWLEQHWSARDNLRSTIRENLNYLKRDSVLKTIQSLVQEHPEATMGLCGYLSQHLTPAEQMQVVQLLSTTESPASH

SEQ ID NO: 5 - protein, 2387 aa, Xenopus sp.
MEGDKEQLPKPPIAEAETPAESDDNLLRTQAEGTTSGQIQDTNSGVNSGTLPPRAASLSKPEQKQLKFAPSRGTEPVNPKPRKQPLSKFILGSSEDNSDDDEFACGSFKTTKRNSQASLGSQTPSLSSLPETESLPTMRSSMSGLHLVKKGRDHKKLDLHRDFTVASPHEFVTRFGGNRVIEKVLIANNGIAAVKCMRSIRRWSYEMFRNERAIRFVVMVTPEDLKANAEYIKMADHYVPVPGGPNNNNYANVELIVDIAKRIPVQAVWAGWGHASENPKLPELLQKQNIAFLGPPSQAMWALGDKIASTIVAQAVGIPTLSWSGDGLLLELKPDDKQQQNIICVPPEVYEKGCVKDADEGLEAAERIGYPVMIKASEGGGGKGIRMAERAEDFPSLFRQVQTEAPGSPIFVMKLAQHARHLEVQILADQYGHAVSLFGRDCSIQRRHQKIIEEAPATVATPSVFEYMEQCAVRLAKMVGYVSAGTVEYLYSEDGSFHFLELNPRLQVEHPCTEMICDVNLPAAQLQISMGVPLYRIKDIRVLYGETPWGDSPICFENPVNAPNPRGHVIAARITSENPDEGFKPSSGTVQELNFRSSKNVWGYFSVAAAGGLHEFADSQFGHCFSWGENREEAISNMVVALKELSIRGDFRTTVEYLIKLLETESFQNNEIDTGWLDHLIAEKVQAEKPDTMLGVVCGALNVADALFQTCMNEFLHCLERGQVLPAASLLNIVDVELISERVKYKLKVARQSLTTYVIILNNSHIEIDVHRLSDGGLLLSYDGNSYTTYMKEEVDRYRITIGNKTCVFEKENDPTVLRSPSTGKLLQYTVEDGSHVNAGECFAEIEVMKMVMALTVQEPGQIHYVKRPGAVLESGCMVAQIDLDDPSKVLQAEPYTGSLLPQQTLPIIGEKLHQVFHSVLENLINVMNGYCLPEPYFTVKIKEWVHKLMKTLRDPSLPLLELQEIMTSVSTRIPPTVERSIRKIMAQYASNITSVLCQFPSQQIASILDSHAATLQRKADREVFFMNTQSIVQLVQRYRSGIRGYMKSVVLDLLRRYLQVETQFQHSHYDKCVIHLREQYKPDMTPVLECIFSHAQVAKKNFLVTMLIDQLCGRDPTLTDELMAILNELTQLSKTEHSKVALRARQVLIASHLPSYELRHNQVESIFLSAIDLYGHQFCPDNLKKLILSETSIFDVLPNFFYHNNQVVRMAALEVYVRRGYIAYELNSLQHHQLRDCTCVVEFQFMLPSSHPNREISPTLSRMSLPISATHLEINRQSSELFMDSGFSPLCQRMGVMVAFNKFEDFTRNFDEVISCFADPPLDSPLFSEVRSSFYDEEDNKNIREEPIHILNVALKSVDRMEDEELVSVFRTFCQSKKNILVDYGLRRITFLIAQQREFPKFFTFRARDEFAEDRIYRHLEPALAFQLELNRMRNFDLNAVPCANHKMHLYLGAAKVAAGIEVTDYRFFVRAIIRHSDLITKEASFEYLQNEGERLLLEAMDELEVAFNNPSVRTDCNHIFLNFVPTVIMDPSKIEESVRSMVMRYGSRLWKLRVLQAEVKINIRLTPTGKAIPIRLFLTNESGYYLDISLYKEVTDPATGQIMFHSYGDKHGHMHGMLINTPYVTKDLLQSKRFQAQSLGTTYVYDFPEMFRQALFKLWRSGEKYPKDILTYTELVLDTQGQLVQLNRLPGGNEVGMVAFKMNLKTPEYPNGREIIVICNDITYKIGSFGPQEDLLFLKTSELARKEGIPRIYIAANSGARIGLAEELRHMFQVAWNNPSDPYKGFKYLYLRPQDYTKISSMNSAHCEHVEDEGESRYVLTDIIGKEEGIGVENLRGSGTIAGESSLAYKEIVTIGMVTCRAIGIGAYLVRLGQRVIQVENSHIILTGASALNKVLGREVYTSNNQLGGVQIMCNNGVSHTMVPDDFEGVYTILQWLSYMPKDNQSPVPVIPPMDPVDRQIEFMPTKAPYDPRWMLAGRPHPTIKGEWQRGFFDHGSFMEIMQRWAQTVVVGRARLGGIPVGVIAVETRSVEMAVPADPANLDSEAKIIQQAGQVWFPDSAFKTAQAIKDFNRERLPLLIFANWRGFSGGMKDMYDQVLKFGAYIVDSLREFKQPVLVYIPPYAELRGGSWVVIDPTINPLYMELYADKDSRGGVLEPEGTVEIRFRKKDLIKTMRRIDPVYTQIVEQLGSPELTEGERKELEKKLRLREEQLLPIYHQVAVRFADLHDTPGRMQEKGVITDILEWKDARSFLYWRLRRLLLEEMVKSEILHANSELSDIHIQSMLRRWFMETEGAVKTYLWDNNQVVVEWLEKHLQEEDEARSAIRENIKYLKKDYALKHIRGLVQANPEVAMDCIVHMTQHITPAQRAQLTRLLSTMDNTPPS

SEQ ID NO: 6 - protein, 8 aa, Unknown
  Description of Unknown ACC2 peptide
GFPLMIKS

SEQ ID NO: 7 - protein, 8 aa, Unknown
  Description of Unknown ACC2 peptide
GFPVMIKS

SEQ ID NO: 8 - protein, 16 aa, Unknown
  Description of Unknown ACC2 peptide
GFPLMIKSASEGGGGK

SEQ ID NO: 9 - protein, 16 aa, Unknown
  Description of Unknown ACC2 peptide
GFPVMIKSASEGGGGK

SEQ ID NO: 10 - protein, 8 aa, Artificial Sequence
  Description of Artificial Sequence Synthetic peptide
DYKDDDDK

SEQ ID NO: 11 - protein, 6 aa, Artificial Sequence
  Description of Artificial Sequence Synthetic 6xHis tag
HHHHHH

SEQ ID NO: 12 - protein, 9 aa, Artificial Sequence
  Description of Artificial Sequence Synthetic peptide
YPYDVPDYA

SEQ ID NO: 13 - DNA, 720 bp, Homo sapiens
atgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactga

SEQ ID NO: 14 - protein, 25 aa, Artificial Sequence
  Description of Artificial Sequence Synthetic peptide
RITSENPDEGFKPSSGTVQELNFRS

SEQ ID NO: 15 - protein, 15 aa, Artificial Sequence
  Description of Artificial Sequence Synthetic peptide
RDFTVASPAEFVTRF

SEQ ID NO: 16 - protein, 15 aa, Artificial Sequence
  Description of Artificial Sequence Synthetic peptide
KEASFEYLQNEGERL

SEQ ID NO: 17 - protein, 12 aa, Artificial Sequence
  Description of Artificial Sequence Synthetic peptide
RAIGIGAYLVRL

SEQ ID NO: 18 - protein, 10 aa, Artificial Sequence
  Description of Artificial Sequence Synthetic peptide
KDMYDQVLKF

SEQ ID NO: 19 - DNA, 42 bp, Artificial Sequence
  Description of Artificial Sequence Synthetic primer
agaagctttgatcatcaatgcaaaaccaattctttctgctgc

SEQ ID NO: 20 - DNA, 42 bp, Artificial Sequence
  Description of Artificial Sequence Synthetic primer
gcagcagaaagaattggttttgcattgatgatcaaagcttct

SEQ ID NO: 21 - DNA, 25 bp, Artificial Sequence
  Description of Artificial Sequence Synthetic primer
ccgcctgcacggcgattctcttggc

SEQ ID NO: 22 - DNA, 25 bp, Artificial Sequence
  Description of Artificial Sequence Synthetic primer
gccaagagaatcgccgtgcaggcgg

SEQ ID NO: 23 - DNA, 29 bp, Artificial Sequence
  Description of Artificial Sequence Synthetic primer
gtaggctgagtctgcgaaccacacctgtc

SEQ ID NO: 24 - DNA, 29 bp, Artificial Sequence
  Description of Artificial Sequence Synthetic primer
gacaggtgtggttcgcagactcagcctac

SEQ ID NO: 25 - DNA, 45 bp, Artificial Sequence
  Description of Artificial Sequence Synthetic primer
ggcagcagaaagaattggttttggattgatgatcaaagcttctga

SEQ ID NO: 26 - DNA, 45 bp, Artificial Sequence
  Description of Artificial Sequence Synthetic primer
tcagaagctttgatcatcaatccaaaaccaattctttctgctgcc

SEQ ID NO: 27 - DNA, 33 bp, Artificial Sequence
  Description of Artificial Sequence Synthetic primer
cagatcgtaggaacccagccgaagtgcagccct

SEQ ID NO: 28 - DNA, 33 bp, Artificial Sequence
  Description of Artificial Sequence Synthetic primer
agggctgcacttcggctgggttcctacgatctg

SEQ ID NO: 29 - DNA, 35 bp, Artificial Sequence
  Description of Artificial Sequence Synthetic primer
gccctcttacgcaaccaaatatgctatgactgtct

SEQ ID NO: 30 - DNA, 35 bp, Artificial Sequence
  Description of Artificial Sequence Synthetic primer
agacagtcatagcatatttggttgcgtaagagggc

SEQ ID NO: 31 - DNA, 21 bp, Artificial Sequence
  Description of Artificial Sequence Synthetic oligonucleotide
cacctgcatctactatctgaa

SEQ ID NO: 32 - DNA, 21 bp, Artificial Sequence
  Description of Artificial Sequence Synthetic oligonucleotide
gtggcttgctatccgggaaat

SEQ ID NO: 33 - DNA, 19 bp, Artificial Sequence
  Description of Artificial Sequence Synthetic primer
acgggctcgggtacgtaag

SEQ ID NO: 34 - DNA, 28 bp, Artificial Sequence
  Description of Artificial Sequence Synthetic primer
cccagttctgattcaggtaatagataca

SEQ ID NO: 35 - DNA, 22 bp, Artificial Sequence
  Description of Artificial Sequence Synthetic primer
gacctgatacgccactgtaacg

SEQ ID NO: 36 - DNA, 20 bp, Artificial Sequence
  Description of Artificial Sequence Synthetic primer
cccggataacaagcaaccat

SEQ ID NO: 37 - DNA, 20 bp, Artificial Sequence
  Description of Artificial Sequence Synthetic primer
atactacgtcaaggagaggt

SEQ ID NO: 38 - DNA, 19 bp, Artificial Sequence
  Description of Artificial Sequence Synthetic primer
tcagcatcaaagtaccaga

SEQ ID NO: 39 - DNA, 24 bp, Artificial Sequence
  Description of Artificial Sequence Synthetic primer
agatgagtatgcctgccgtgtgaa

SEQ ID NO: 40 - DNA, 24 bp, Artificial Sequence
  Description of Artificial Sequence Synthetic primer
tgctgcttacatgtctcgatccca

SEQ ID NO: 41 - DNA, 21 bp, Artificial Sequence
  Description of Artificial Sequence Synthetic primer
atcccgtaccttcttctactg

SEQ ID NO: 42 - DNA, 21 bp, Artificial Sequence
  Description of Artificial Sequence Synthetic primer
cccaaacataagccttcactg

SEQ ID NO: 43 - DNA, 20 bp, Artificial Sequence
  Description of Artificial Sequence Synthetic primer
ctctgaccatgttcgttctc

SEQ ID NO: 44 - DNA, 20 bp, Artificial Sequence
  Description of Artificial Sequence Synthetic primer
atcttcatcacctccatctc

SEQ ID NO: 45 - DNA, 20 bp, Artificial Sequence
  Description of Artificial Sequence Synthetic primer
gattttgctgtcggtcttgg

SEQ ID NO: 46 - DNA, 20 bp, Artificial Sequence
  Description of Artificial Sequence Synthetic primer
ctcttgctgcctgaatgtga

SEQ ID NO: 47 - DNA, 20 bp, Artificial Sequence
  Description of Artificial Sequence Synthetic primer
attcccaccgcggaaggtgc

SEQ ID NO: 48 - DNA, 20 bp, Artificial Sequence
  Description of Artificial Sequence Synthetic primer
gcagcctgggggcagtcttg

SEQ ID NO: 49 - DNA, 21 bp, Artificial Sequence
  Description of Artificial Sequence Synthetic primer
tcattgtggaagcagataccc

SEQ ID NO: 50 - DNA, 22 bp, Artificial Sequence
  Description of Artificial Sequence Synthetic primer
cagctccgtcaccaattaaaac

SEQ ID NO: 51 - DNA, 19 bp, Artificial Sequence
  Description of Artificial Sequence Synthetic primer
tgtggaaggagtttcgcag

SEQ ID NO: 52 - DNA, 21 bp, Artificial Sequence
  Description of Artificial Sequence Synthetic primer
gggatatgctggtgttctcag

SEQ ID NO: 53 - DNA, 21 bp, Artificial Sequence
  Description of Artificial Sequence Synthetic primer
ccacttgctgtgccaaatgga

SEQ ID NO: 54 - DNA, 21 bp, Artificial Sequence
  Description of Artificial Sequence Synthetic primer
gaaggactttaccttccagga

SEQ ID NO: 55 - DNA, 24 bp, Artificial Sequence
  Description of Artificial Sequence Synthetic primer
gattgtccgtaacattctcatcga

SEQ ID NO: 56 - DNA, 21 bp, Artificial Sequence
  Description of Artificial Sequence Synthetic primer
tgtcttgagccgctctgagat

SEQ ID NO: 57 - DNA, 24 bp, Artificial Sequence
  Description of Artificial Sequence Synthetic primer
accctgctgggtttatggatgtca

SEQ ID NO: 58 - DNA, 24 bp, Artificial Sequence
  Description of Artificial Sequence Synthetic primer
tacgatgaacagcaaagcgaccct

SEQ ID NO: 59 - DNA, 20 bp, Artificial Sequence
  Description of Artificial Sequence Synthetic primer
cagaccgcaggaatccacat

SEQ ID NO: 60 - DNA, 24 bp, Artificial Sequence
  Description of Artificial Sequence Synthetic primer
ttcagcatcgaagtaccagacagt

SEQ ID NO: 61 - protein, 18 aa, Artificial Sequence
  Description of Artificial Sequence Synthetic peptide
ERIGFPLMIKASEGGGGK

SEQ ID NO: 62 - protein, 15 aa, Artificial Sequence
  Description of Artificial Sequence Synthetic peptide
AGQVWFPDSAYKTAQ

SEQ ID NO: 63 - protein, 15 aa, Artificial Sequence
  Description of Artificial Sequence Synthetic peptide
ARLELDDPSKVHPAE

SEQ ID NO: 64 - protein, 21 aa, Artificial Sequence
  Description of Artificial Sequence Synthetic peptide
KRIPVQAVWAGWGHASENPKL

SEQ ID NO: 65 - protein, 21 aa, Artificial Sequence
  Description of Artificial Sequence Synthetic peptide
KDVDEGLEAAERIGFPLMIKA

SEQ ID NO: 66 - protein, 18 aa, Artificial Sequence
  Description of Artificial Sequence Synthetic peptide
KIIQQAGQVWFPDSAYKT

SEQ ID NO: 67 - protein, 18 aa, Homo sapiens
ERIGFPLMIKASEGGGGK

SEQ ID NO: 68 - protein, 18 aa, Rattus sp.
EKVGFPLMIKASEGGGGK

SEQ ID NO: 69 - protein, 18 aa, Mus sp.
EKIGFPLMIKASEGGGGK

SEQ ID NO: 70 - protein, 18 aa, Caenorhabditis elegans
HNIGFPLMIKASEGGGGK

SEQ ID NO: 71 - protein, 18 aa, Drosophila sp.
NKIGFPVMIKASEGGGGK

SEQ ID NO: 72 - protein, 18 aa, Saccharomyces cerevisiae
KRIGFPVMIKASEGGGGK

SEQ ID NO: 73 - protein, 19 aa, Artificial Sequence
  Description of Artificial Sequence Synthetic peptide
DVDEGLEAAERIGFPLMIK

(19) United States
(12) Patent Application Publication
(10) Pub. No.: US 2018/0306788 A1
(43) Pub. Date: Oct. 25, 2018

(54) COMPOSITIONS AND METHODS FOR MODULATING HYDROXYLATION OF ACC2 BY PHD3

(71) Applicant: President and Fellows of Harvard College, Cambridge, MA (US)

(72) Inventors: Marcia C. Haigis, Winchester, MA (US); Natatie J. German, Cambridge, MA (US)

(21) Appl. No.: 15/564,956

(22) PCT Filed: Apr. 7, 2016
(86) PCT No.: PCT/US2016/026461

§ 371 Date: Oct. 6, 2017

Related U.S. Application Data
(60) Provisional application No. 62/144,165, filed on Apr. 7, 2015.

(51) Int. Cl. G01N 33/574; A61K 31/7105; A61P 35/00; A61P 3/04

(52) U.S. Cl. CPC G01N 33/574; A61K 31/7105; A61P 35/00; G01N 2800/52; G01N 2800/60; G01N 2800/7028; A61P 3/04

(57) ABSTRACT

Compositions and methods useful for treating a number of human disorders including, but not limited to, cancer, cardiovascular disease, obesity, and metabolic disorders are provided. For example, the disclosure features compositions and methods for modulating the hydroxylation of ACC2 by PHD3 in vitro or in vivo. Also provided are methods for monitoring and/or detecting the expression of PHD3 and/or levels of ACC2 hydroxylation, which are useful for, inter alia, determining whether a cancer cell is sensitive to glycolytic pathway inhibitors or inhibitors of fatty acid metabolism.

### RELATED APPLICATION

This application is the U.S. National Stage of International Patent Application No. PCT/US2016/026461, filed Apr. 7, 2016, which claims the benefit of priority to U.S. Provisional Patent Application Ser. No. 62/144,165, filed Apr. 7, 2015, the contents of both of which are hereby incorporated herein by reference in their entirety.

### GOVERNMENT SUPPORT

This invention was made in part with government support under Grant No. T32 GM007306 awarded by the National Institutes of Health. The government may have certain rights in the invention.

### SEQUENCE LISTING

The instant application contains a Sequence Listing which has been submitted electronically in ASCII format and is hereby incorporated by reference in its entirety. Said ASCII copy, created on May 13, 2016, is named HMV-246.25_SL.txt and is 104,982 bytes in size.

### BACKGROUND

Glycolysis and glutaminolysis are fundamentally altered in cancer metabolism to drive biosynthetic pathways, such as lipid synthesis (7-9). However, a substantial subset of cancers, for reasons largely not understood, have a high capacity and a preference for fat oxidation (5).

### SUMMARY

The disclosure is based, at least in part, on the discovery that prolyl hydroxylase 3 (PHD3) interacts with, and hydroxylates, acetyl-CoA carboxylase 2 (ACC2). PHD3 hydroxylation of ACC2 activates ACC2 to repress long chain fatty acid oxidation. Thus, PHD3 is a regulator of fatty acid oxidation. Accordingly, the disclosure features a number of compositions, kits, and applications based on these discoveries. For example, detecting or monitoring the level of ACC2 hydroxylation is useful for applications, such as, but not limited to, methods for determining whether a cancer is more amenable to treatment with a FAO inhibitor or a glycolytic pathway inhibitor. Moreover, modulating hydroxylation of ACC2 is useful for treating a variety of conditions associated with fatty acid imbalance including, e.g., cardiovascular disease, metabolic disorders, obesity, diabetes, and the like.

The disclosure is also based on the discovery that repression of PHD3 expression by cancer cells is a mechanism by which such cells can amplify fatty acid consumption. While the disclosure is not limited by any particular theory or mechanism of action, elevated fatty acid catabolism can promote survival in certain cancers, by serving as a source of ATP or NADPH, a molecule with antioxidant functions generated upon channeling acetyl-CoA towards citrate-cycling reactions, or alternatively by maintaining the quality of the mitochondrial membrane. Thus, detecting or monitoring the level of PHD3 expression is useful for applications, such as, but in no way limited to, methods for determining whether a cancer is more sensitive to treatment with a FAO inhibitor or a glycolytic pathway inhibitor, and for treating cancer.

Thus, in one aspect, the disclosure features a method for treating a subject having a cancer comprising cancer cells with reduced PHD3 expression. The method comprises administering to the subject an inhibitor of fatty acid metabolism, such as a fatty acid oxidation (FAO) inhibitor, in an amount effective to treat the cancer.

In another aspect, the disclosure features a method for treating a subject having a cancer, the method comprising administering to the subject an inhibitor of fatty acid metabolism, such as a fatty acid oxidation (FAO) inhibitor, in an amount effective to treat the cancer, wherein the cancer has been identified as comprising cancer cells with reduced PHD3 expression.

In another aspect, the disclosure features a method for treating a subject having a cancer, which method includes: receiving the results of a test determining that the subject's cancer comprises cancer cells with reduced PHD3 expression; and ordering administration of an effective amount of an inhibitor of fatty acid metabolism, such as a fatty acid oxidation (FAO) inhibitor, to the subject.

In yet another aspect, the disclosure features a method for treating a subject having a cancer. The method comprises: requesting a test, or the results of a test, determining that the subject's cancer comprises cancer cells with reduced PHD3 expression; and ordering administration of an effective amount of an inhibitor of fatty acid metabolism, such as a fatty acid oxidation (FAO) inhibitor, to the subject.

In some embodiments, the cancer is a prostate cancer. In some embodiments, the cancer is a glioblastoma. In some embodiments, the cancer is of hematological origin, e.g., acute myeloid leukemia.

In some embodiments, the subject is a human.

In some embodiments, PHD3 expression by the cancer cells is less than or equal to 90% of normal cells of the same histological type from which the cancer cells are derived. In some embodiments, PHD3 expression by the cancer cells is less than or equal to 80% of normal cells of the same histological type from which the cancer cells are derived. In some embodiments, PHD3 expression by the cancer cells is less than or equal to 70% of normal cells of the same histological type from which the cancer cells are derived. In some embodiments, PHD3 expression by the cancer cells is less than or equal to 50% of normal cells of the same histological type from which the cancer cells are derived. In some embodiments, PHD3 expression by the cancer cells is less than or equal to 25% of normal cells of the same histological type from which the cancer cells are derived. In some embodiments, PHD3 expression by the cancer cells is less than or equal to 15% of normal cells of the same histological type from which the cancer cells are derived.

In some embodiments, any of the methods described herein further comprise determining whether the cancer cells have reduced PHD3 expression.

In some embodiments, the FAO inhibitor is a carnitine palmitoyl transferase (CPT-I) inhibitor, such as etomoxir, oxfenicine, or perhexiline. In some embodiments, the FAO inhibitor is a 3-ketoacyl-coenzyme A thiolase (3-KAT) inhibitor, such as trimetazidine or ranolazine. In some embodiments, the FAO inhibitor is a mitochondrial thiolase inhibitor, such as 4-bromocrotonic acid.

In another aspect, the disclosure features a method for treating a subject having a cancer comprising cancer cells with elevated PHD3 expression. The method comprises administering to the subject a glycolytic pathway inhibitor in an amount effective to treat the cancer.

In another aspect, the disclosure features a method for treating a subject having a cancer, which method comprises administering to the subject a glycolytic pathway inhibitor in an amount effective to treat the cancer, wherein the cancer has been identified as comprising cancer cells with elevated PHD3 expression.

In another aspect, the disclosure features a method for treating a subject having a cancer. The method comprises: receiving the results of a test determining that the subject's cancer comprises cancer cells with reduced PHD3 expression; and administering or ordering administration of an effective amount of a glycolytic pathway inhibitor to the subject.

In another aspect, the disclosure features a method for treating a subject having a cancer, which method comprises: requesting a test, or the results of a test, determining that the subject's cancer comprises cancer cells with elevated PHD3 expression; and ordering administration of an effective amount of a glycolytic pathway inhibitor to the subject.

In some embodiment, the cancer is a pancreatic cancer. In some embodiments, the cancer is a kidney cancer or bladder cancer. In some embodiments, the cancer is a melanoma, a lung cancer, a follicular lymphoma, a breast cancer, a colorectal cancer, or an ovarian cancer.

In some embodiments, the subject is a human.

In some embodiments, PHD3 expression by the cancer cells is at least 20% greater than that of normal cells of the same histological type from which the cancer cells are derived. In some embodiments, PHD3 expression by the cancer cells is at least 50% greater than that of normal cells of the same histological type from which the cancer cells are derived. In some embodiments, PHD3 expression by the cancer cells is at least 75% greater than that of normal cells of the same histological type from which the cancer cells are derived. In some embodiments, PHD3 expression by the cancer cells is at least 100% greater than that of normal cells of the same histological type from which the cancer cells are derived. In some embodiments, PHD3 expression by the cancer cells is at least 2.5 fold greater than that of normal cells of the same histological type from which the cancer cells are derived. In some embodiments, PHD3 expression by the cancer cells is at least 5 fold greater than that of normal cells of the same histological type from which the cancer cells are derived.

In some embodiments, any of the methods described herein can further comprise determining whether the cancer cells have elevated PHD3 expression.

In some embodiments, the glycolytic pathway inhibitor is a hexokinase inhibitor, such as 2-deoxyglucose, 3-bromopyruvate, or lonidamine. In some embodiments, the glycolytic pathway inhibitor is a transketolase inhibitor, such as oxythiamine. In some embodiments, the glycolytic pathway inhibitor is imatinib. In some embodiments, the glycolytic pathway inhibitor is a glucose transporter (GLUT) inhibitor. In some embodiments, the glycolytic pathway inhibitor is a phosphofructokinase (PFK) inhibitor. In some embodiments, the glycolytic pathway inhibitor is a glyceraldehyde-3-phosphate dehydrogenase (GAPDH) inhibitor. In some embodiments, the glycolytic pathway inhibitor is a pyruvate kinase (PK) inhibitor. In some embodiments, the glycolytic pathway inhibitor is a lactate dehydrogenase (LDH) inhibitor.

In yet another aspect, the disclosure features a method for treating a subject having a cancer characterized by cancer cells having a reduced level of hydroxylation of ACC2 at proline 450 relative to SEQ ID NO:2. The method comprises administering to the subject a fatty acid oxidation (FAO) inhibitor in an amount effective to treat the cancer.

In another aspect, the disclosure features a method for treating a subject having a cancer, which method comprises administering to the subject a fatty acid oxidation (FAO) inhibitor in an amount effective to treat the cancer, wherein the cancer has been identified as comprising cancer cells having a reduced level of hydroxylation of ACC2 at proline 450 relative to SEQ ID NO:2.

In another aspect, the disclosure features a method for treating a subject having a cancer. The method comprises: receiving the results of a test determining that the subject's cancer comprises cancer cells having a reduced level of hydroxylation of ACC2 at proline 450 relative to SEQ ID NO:2; and ordering administration of an effective amount of a fatty acid oxidation (FAO) inhibitor to the subject.

In yet another aspect, the disclosure features a method for treating a subject having a cancer. The method comprises: requesting a test, or the results of a test, determining that the subject's cancer comprises cancer cells having a reduced level of hydroxylation of ACC2 at proline 450 relative to SEQ ID NO:2; and administering or ordering administration of an effective amount of a fatty acid oxidation (FAO) inhibitor to the subject.

In some embodiments, the cancer is a prostate cancer. In some embodiments, the cancer is a glioblastoma. In some embodiments, the cancer is of hematological origin, e.g., acute myeloid leukemia.

In some embodiments, the subject is a human.

In some embodiments, the level of hydroxylation of ACC2 at proline 450 by the cancer cells is less than or equal to 90% of normal cells of the same histological type from which the cancer cells are derived. In some embodiments, the level of hydroxylation of ACC2 at proline 450 by the cancer cells is less than or equal to 80% of normal cells of the same histological type from which the cancer cells are derived. In some embodiments, the level of hydroxylation of ACC2 at proline 450 by the cancer cells is less than or equal to 70% of normal cells of the same histological type from which the cancer cells are derived. In some embodiments, the level of hydroxylation of ACC2 at proline 450 by the cancer cells is less than or equal to 50% of normal cells of the same histological type from which the cancer cells are derived. In some embodiments, the level of hydroxylation of ACC2 at proline 450 by the cancer cells is less than or equal to 25% of normal cells of the same histological type from which the cancer cells are derived. In some embodiments, the level of hydroxylation of ACC2 at proline 450 by the cancer cells is less than or equal to 15% of normal cells of the same histological type from which the cancer cells are derived.

In another aspect, the disclosure features a method for treating a subject having a cancer comprising cancer cells with an elevated level of hydroxylation of ACC2 at proline 450 relative to SEQ ID NO:2, the method comprising administering to the subject a glycolytic pathway inhibitor in an amount effective to treat the cancer.

In another aspect, the disclosure features a method for treating a subject having a cancer, the method comprising administering to the subject a glycolytic pathway inhibitor in an amount effective to treat the cancer, wherein the cancer has been identified as comprising cancer cells with an elevated level of hydroxylation of ACC2 at proline 450 relative to SEQ ID NO:2. In another aspect, the disclosure features a method for treating a subject having a cancer, the method comprising: receiving the results of a test determining that the subject's cancer comprises cancer cells with an elevated level of hydroxylation of ACC2 at proline 450 relative to SEQ ID NO:2; and administering or ordering administration of an effective amount of a glycolytic pathway inhibitor to the subject.

In yet another aspect, the disclosure features a method for treating a subject having a cancer, the method comprising: requesting a test, or the results of a test, determining that the subject's cancer comprises cancer cells with an elevated level of hydroxylation of ACC2 at proline 450 relative to SEQ ID NO:2; and administering or ordering administration of an effective amount of a glycolytic pathway inhibitor to the subject.

In some embodiment, the cancer is a pancreatic cancer. In some embodiments, the cancer is a kidney cancer or bladder cancer. In some embodiments, the cancer is a melanoma, a lung cancer, a follicular lymphoma, a breast cancer, a colorectal cancer, or an ovarian cancer.

In some embodiments, the subject is a human.

In some embodiments, the level of hydroxylation of ACC2 at proline 450 by the cancer cells is at least 20% greater than that of normal cells of the same histological type from which the cancer cells are derived. In some embodiments, the level of hydroxylation of ACC2 at proline 450 by the cancer cells is at least 50% greater than that of normal cells of the same histological type from which the cancer cells are derived. In some embodiments, the level of hydroxylation of ACC2 at proline 450 by the cancer cells is at least 75% greater than that of normal cells of the same histological type from which the cancer cells are derived. In some embodiments, the level of hydroxylation of ACC2 at proline 450 by the cancer cells is at least 100% greater than that of normal cells of the same histological type from which the cancer cells are derived. In some embodiments, the level of hydroxylation of ACC2 at proline 450 by the cancer cells is at least 2.5 fold greater than that of normal cells of the same histological type from which the cancer cells are derived. In some embodiments, the level of hydroxylation of ACC2 at proline 450 by the cancer cells is at least 5 fold greater than that of normal cells of the same histological type from which the cancer cells are derived.

In yet another aspect, the disclosure features an isolated antibody, or fragment thereof, that preferentially binds to an ACC2 polypeptide when hydroxylated at proline 450 relative to SEQ ID NO:2 over the ACC2 polypeptide when not hydroxylated at proline 450 relative to SEQ ID NO:2.

In some embodiments, the isolated antibody, or fragment thereof, only binds to an ACC2 polypeptide when hydroxylated at proline 450 relative to SEQ ID NO:2.

In another aspect, the disclosure features an isolated antibody, or fragment thereof, that only binds to an ACC2 polypeptide when not hydroxylated at proline 450 relative to SEQ ID NO:2.

In another aspect, the disclosure features an isolated antibody, or fragment thereof, that specifically binds to an ACC2 polypeptide that is hydroxylated at proline 450 relative to SEQ ID NO:2, wherein the antibody specifically binds to an epitope that is within the amino acid sequence of any one of SEQ ID NOs: 6-9.

In some embodiments, the fragment is a Fab, Fv, single-chain (scFv), Fab′, or F(ab′)₂.

In some embodiments, the antibody is a minibody or domain antibody.

In some embodiments, the antibody is a whole antibody.

In another aspect, the disclosure features a method for detecting P450-hydroxylated ACC2 in a biological sample, the method comprising: (a) contacting a biological sample with at least one of any of the antibodies described herein under conditions suitable for formation of a complex between the antibody and ACC2 that is hydroxylated at proline 450 relative to SEQ ID NO:2, if such hydroxylated ACC2 is present in the biological sample; and (b) detecting the presence of the complex in the biological sample, wherein the presence of the complex indicates the presence of hydroxylated ACC2 in the biological sample.

In another aspect, the disclosure features a method for detecting P450-hydroxylated ACC2 in a biological sample, the method comprising: (a) contacting a biological sample with at least one of any of the antibodies described herein under conditions suitable for formation of a complex between the antibody and ACC2 that is hydroxylated at proline 450 relative to SEQ ID NO:2, if such hydroxylated ACC2 is present in the biological sample; (b) contacting the complex of (a) with a detection reagent; and (c) detecting the presence or amount of the detection reagent as a measure of the presence or amount of the complex in the biological sample, wherein the presence of the complex indicates the presence of P450-hydroxylated ACC2 in the biological sample.

In yet another aspect, the disclosure features a method for detecting P450-hydroxylated ACC2 in a biological sample, the method comprising: (a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample; and (b) detecting the presence or amount of the detection reagent as a measure of the presence or amount of the complex in the biological sample, wherein the presence of the complex indicates the presence of hydroxylated ACC2 in the biological sample.

In another aspect, the disclosure features a nucleic acid encoding any one of the antibodies described herein. The disclosure also features a vector or an expression vector comprising the nucleic acid. Also featured is a cell (e.g., a host cell) comprising the vector, expression vector, or nucleic acid. The disclosure further features a method for producing an antibody by culturing the cell or a plurality of the cells under conditions suitable for expression of the antibody and, optionally, isolating the antibody from the cell(s) or from the medium in which the cell(s) is cultured.

In another aspect, the disclosure features a kit for the detection of hydroxylated ACC2 in a biological sample. The kit comprises: (a) at least one of any of the antibodies described herein or one of the nucleic acids, vectors expression vectors, or cells, and (b) at least one secondary reagent. The at least one secondary reagent can be, e.g., an antibody that binds to the at least one antibody of (a).

In another aspect, the disclosure features an isolated polypeptide comprising at least 10 consecutive amino acids of SEQ ID NO:2, but no more than 2000 consecutive amino acids of SEQ ID NO:2, wherein the polypeptide comprises proline 450 of SEQ ID NO:2.

In another aspect, the disclosure features an isolated polypeptide comprising at least 10 consecutive amino acids of SEQ ID NO:2, including proline 450 of SEQ ID NO:2, wherein the polypeptide comprises at most 98% of SEQ ID NO:2.

In another aspect, the disclosure features an isolated polypeptide comprising at least 10 consecutive amino acids of SEQ ID NO:2 inclusive of the proline residue at position 450 of SEQ ID NO:2, wherein the proline residue at position 450 is mutated, modified, or deleted.

In another aspect, the disclosure features a polypeptide comprising: (i) the amino acid sequence depicted in SEQ ID NO:2, wherein the proline residue at position 450 is mutated, modified, or deleted; (ii) a variant of the amino acid sequence depicted in SEQ ID NO:2 having not more than 100 amino acid substitutions, deletions, or insertions, and wherein the proline residue at position 450 is mutated, modified, or deleted; or (iii) an amino acid sequence that is at least 80% identical to any one of the amino acid sequences depicted in SEQ ID NO:2, wherein the proline residue at position 450 is mutated, modified, or deleted.

In some embodiments, any of the polypeptides described herein can be hydroxylated, e.g., the proline residue at position 450 is hydroxylated.

In some embodiments, any one of the polypeptides described herein further comprises a heterologous moiety.

In another aspect, the disclosure features a nucleic acid encoding any one of the polypeptides described herein. The disclosure also features a vector or an expression vector comprising the nucleic acid. Also featured is a cell (e.g., a host cell) comprising the vector, expression vector, or nucleic acid. The disclosure further features a method for producing a polypeptide by culturing the cell or a plurality of the cells under conditions suitable for expression of the polypeptide and, optionally, isolating the polypeptide from the cell(s) or from the medium in which the cell(s) is cultured.

In another aspect, the disclosure features a method for determining whether a cancer is susceptible to a fatty acid oxidation inhibitor. The method comprises: (a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and (b) detecting the presence or amount of the detection reagent as a measure of the presence or amount of the complex in the biological sample, wherein a reduced level of ACC2 hydroxylated at proline 450, relative to a control level, indicates that the cancer is susceptible to a fatty acid oxidation inhibitor.

In another aspect, the disclosure features a method for determining whether a cancer patient will benefit from treatment with a fatty acid oxidation inhibitor, the method comprising: (a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and (b) detecting the presence or amount of the detection reagent as a measure of the presence or amount of the complex in the biological sample, wherein a reduced level of ACC2 hydroxylated at proline 450, relative to a control level, indicates that the cancer patient will benefit from treatment with a fatty acid oxidation inhibitor.

In another aspect, the disclosure features a method for determining whether a cancer is susceptible to a glycolytic pathway inhibitor, the method comprising: (a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and (b) detecting the presence or amount of the detection reagent as a measure of the presence or amount of the complex in the biological sample, wherein an elevated level of ACC2 hydroxylated at proline 450, relative to a control level, indicates that the cancer is susceptible to a glycolytic pathway inhibitor.

In another aspect, the disclosure features a method for determining whether a cancer patient will benefit from treatment with a glycolytic pathway inhibitor, the method comprising: (a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and (b) detecting the presence or amount of the detection reagent as a measure of the presence or amount of the complex in the biological sample, wherein an elevated level of ACC2 hydroxylated at proline 450, relative to a control level, indicates that the cancer patient will benefit from treatment with a glycolytic pathway inhibitor.

In some embodiments, any of the methods described herein can further comprise communicating to a subject (e.g., a patient) or a medical professional (e.g., a doctor) the results of a determination as to whether the subject will benefit from a given therapy. In some embodiments, any of the methods described herein can comprise receiving a request (e.g., from a patient, medical professional or insurance provider) to perform a test to determine whether a subject will benefit from a given therapy.

In yet another aspect, the disclosure features a method for increasing fatty acid oxidation by a cell, the method comprising contacting the cell with a compound that inhibits the hydroxylation of ACC2 (e.g., at proline 450 relative to SEQ ID NO:2) by PHD3 in an amount effective to increase fatty acid oxidation by the cell.

In another aspect, the disclosure features a method for increasing fatty acid oxidation in a subject in need thereof, the method comprising administering to the subject a compound that inhibits the hydroxylation of ACC2 (e.g., at proline 450 relative to SEQ ID NO:2) by PHD3 in an amount effective to increase fatty acid oxidation in the subject.

In another aspect, the disclosure features a method for promoting weight loss in a subject, the method comprising administering to the subject a compound that inhibits the hydroxylation of ACC2 (e.g., at proline 450 relative to SEQ ID NO:2) by PHD3 in an amount effective to promote weight loss in the subject.

In another aspect, the disclosure features a method for treating cardiovascular disease in a subject, the method comprising administering to the subject a compound that inhibits the hydroxylation of ACC2 (e.g., at proline 450 relative to SEQ ID NO:2) by PHD3 in an amount effective to treat the cardiovascular disease in the subject.

In another aspect, the disclosure features a method for treating a subject afflicted with a metabolic syndrome, diabetes, obesity, atherosclerosis, or cardiovascular disease, the method comprising administering to the subject a compound that inhibits the hydroxylation of ACC2 at proline 450 relative to SEQ ID NO:2 by PHD3 in an amount effective to treat the metabolic syndrome, diabetes, obesity, atherosclerosis, or cardiovascular disease.

In some embodiments of any of the methods described herein, the subject is obese or is overweight. In some embodiments of any of the methods described herein, the subject has coronary artery disease. In some embodiments of any of the methods described herein, the subject has diabetes.

In yet another aspect, the disclosure features a method for treating or delaying the onset of an obesity-related disorder in a subject, the method comprising administering to the subject a compound that inhibits the hydroxylation of ACC2 (e.g., at proline 450 relative to SEQ ID NO:2) by PHD3 in an amount effective to treat or delay the onset of an obesity-related disorder in the subject.

In another aspect, the disclosure features a method for treating a subject having a cancer, the method comprising: administering to the subject an inhibitor of PHD3 to thereby sensitize the cancer to inhibition of fatty acid metabolism (e.g., a fatty acid oxidation (FAO) inhibitor); and administering to the subject an effective amount of inhibitor of fatty acid metabolism to treat the cancer, wherein the effective amount of the inhibitor of fatty acid metabolism is lower than the amount effective to treat the cancer in the absence of PHD3 inhibition.

In some embodiments, the inhibitor of PHD3 is administered first in time and the FAO inhibitor administered second in time. In some embodiments, the inhibitor of PHD3 and the FAO inhibitor are administered concurrently.

In some embodiments, the inhibitor of PHD3 binds to and inhibits the activity of PHD3. For example, the inhibitor of PHD3 can be, e.g., a small molecule, a macrocycle compound, a polypeptide, a nucleic acid, or a nucleic acid analog.

In some embodiments, the inhibitor of PHD3 reduces the expression or stability of an mRNA encoding PHD3 protein. The compound can be, e.g., an antisense oligonucleotide, an siRNA, an shRNA, or a ribozyme.

In some embodiments, the cancer is a prostate cancer, a glioblastoma, or a cancer is of hematological origin.

In some embodiments, PHD3 expression by the cancer cells is less than or equal to 90% of normal cells of the same histological type from which the cancer cells are derived.

In some embodiments, any one of the methods can further comprise determining whether the cancer cells have reduced PHD3 expression.

In yet another aspect, the disclosure features a method for identifying a modulator of PHD3 activity, the method comprising: contacting, in the presence of a substrate ACC2 protein, a PHD3 protein or an enzymatically-active fragment thereof with a candidate compound; and detecting hydroxylation of the substrate ACC2 protein by the PHD3 protein or enzymatically-active fragment thereof, wherein a difference in the amount of hydroxylation of the substrate ACC2 protein by the PHD3 protein or enzymatically-active fragment thereof in the presence of the candidate compound, as compared to the amount of hydroxylation of the substrate ACC2 protein by the PHD3 protein or enzymatically-active fragment thereof in the absence of the candidate compound, indicates that the candidate compound modulates PHD3 activity.

In another aspect, the disclosure features a method of screening for candidate compounds which are capable of modulating the activity of a PHD3 protein or enzymatically-active fragment thereof to hydroxylate a substrate ACC2 protein, the method comprising determining whether at least one candidate compound has the property of modulating the activity of a PHD3 protein or enzymatically-active fragment thereof to hydroxylate a substrate ACC2 protein under conditions in which the PHD3 protein or enzymatically-active fragment thereof is capable of hydroxylating the substrate ACC2 protein in the absence of the candidate compound. In some embodiments, the method comprises: (a) contacting at least one candidate compound, a substrate ACC2 protein and the PHD3 protein or enzymatically-active fragment thereof under conditions in which the PHD3 protein or enzymatically-active fragment thereof is capable of hydroxylating position P450 of the substrate ACC2 protein in the absence of the candidate compound; (b) determining whether the candidate compound modulates the hydroxylation of the substrate ACC2 protein at position P450 by the PHD3 protein or enzymatically-active fragment thereof; and (c) identifying the candidate compound as a modulator of PHD3 protein if the compound modulates the hydroxylation of the substrate ACC2 protein at position P450 by the PHD3 protein or enzymatically-active fragment thereof.

In some embodiments of any of the methods described herein, the candidate compound inhibits hydroxylation of the substrate ACC2 protein by the PHD3 protein or enzymatically-active fragment thereof.

In some embodiments, the contacting occurs in a cell. For example, in some embodiments, the cell comprises one or both of: (a) a transgene encoding the substrate ACC2 protein and (b) a transgene encoding the PHD3 protein or enzymatically-active fragment thereof.

In some embodiments, the contacting occurs in vitro (e.g., using recombinant proteins).

In another aspect, the disclosure features a method of identifying an agent which inhibits hydroxylation of a substrate ACC2 protein by a PHD3 protein or enzymatically-active fragment thereof, the method comprising: introducing into a cell that expresses a substrate ACC2 protein a vector that expresses a PHD3 protein or enzymatically-active fragment thereof, contacting the cell with a test compound under conditions in which P450 in the substrate ACC2 protein is hydroxylated by PHD3 in the absence of the test substance, and determining hydroxylation of the substrate, wherein a decrease in the hydroxylation of P450 of the substrate ACC2 protein in the presence of the test compound as compared to the hydroxylation of P450 of the substrate ACC2 protein in the absence of the test compound identifies the test substance as an agent that inhibits hydroxylation of ACC2 by PHD3.

“Polypeptide,” “peptide,” and “protein” are used interchangeably and mean any peptide-linked chain of amino acids, regardless of length or post-translational modification. As noted below, the polypeptides described herein can be, e.g., wild-type proteins, functional fragments of the wild-type proteins, or variants of the wild-type proteins or fragments.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for diagnosing a and treating a patient with cancer, will be apparent from the following description, the examples, and from the claims.

### BRIEF DESCRIPTION OF THE FIGURES

FIG. 1 includes 10 panels (Panels A-J), which show that PHD3 interacts with ACC and represses fatty acid oxidation (FAO). Panel A is an immunoblot showing the interaction between ACC and PHD3. An expression vector encoding a hemagluttanin (HA)-tagged PHD1, 2, or 3, or an empty expression vector (as a control), was transfected in 293T cells. HA-tagged proteins were immunoprecipitated with an anti-HA antibody affinity resin, and interactions were detected by immunoblotting for ACC. Panel B is a bar graph depicting basal fatty acid oxidation of palmitate by 293T cells transiently overexpressing HA-PHD2, HA-PHD3, or only transformed with the empty vector (n=3). Panel C is a bar graph depicting PHD1, 2 and 3 gene expression in 293T cells stably expressing shRNA against PHD3 (shPHD3.1 and shPHD3.2) or non-targeting control (shControl). Panel D is a bar graph depicting palmitate oxidation by 293T cells stably expressing shRNA against PHD3 or non-targeting control (n=4). Panel E is a bar graph depicting palmitate oxidation in HepG2 cells with PHD3 knockdown (n=3). shPHD3.2 was used here and for all further studies with one shRNA against PHD3. Panel F is a bar graph depicting the impact of PHD3 levels on long chain versus short chain FAO. Oxidation of long chain palmitic acid and short chain hexanoic acid was assessed in 293T cells stably expressing shPHD3 or non-targeting control shRNA (n=3). Panel G is a photograph of an immunoblot showing HIF1α and HIF2α levels in 293T cells with PHD3 knockdown or control. Bands representing HIF1/2α were made more visible following 4 hour treatment with 250 μM CoCl₂. Panel H is a bar graph depicting palmitate oxidation in 786-O VHL−/− cells, which have constitutively stabilized HIF. Cells were transiently transfected with Dharmacon siGENOME SMARTpool EGLN3 siRNA (siPHD3) or control Non-Targeting siRNA Pool #2 (siControl), and FAO was assess 48 hour later (n=3). Panels I and J are a pair of bar graphs depicting the effects of PHD3 levels on palmitate oxidation in complete media in ARNT −/− cells, which have constitutively inactive HIF. FAO was assessed in these cells following (Panel I) transient transfection with siPHD3 or siControl, as above, and (Panel J) transient transfection with human HA-PHD3 or vector (n=3). *<0.05, ***<0.001. Error bars indicate SEM.

FIG. 2 includes 10 panels (Panels A-J), which show that PHD3 modifies ACC2 by site-specific prolyl-hydroxylation. Panel A is a photograph of an immunoblot showing endogenous ACC hydroxylation was measured in 293T cells transiently overexpressing HA-PHD3 or vector. ACC was immunoprecipitated by ACC antibody and Protein G affinity resin. Hydroxylation was detected by immunoblot with hydroxyproline (OH-Pro) antibody. Panel B is a photograph of an immunoblot showing endogenous ACC hydroxylation was measured in 293T cell transiently overexpressing wild type PHD3 or two catalytically inactive PHD3 mutants (R206K and H196A). Hydroxyproline was assessed by immunoblot, as above. Panel C is a photograph of an immunoblot showing endogenous ACC hydroxylation was measured in 293T cells following stable PHD3 knockdown by two different shRNA or non-targeting control. Hydroxylation was assessed by immunoblot, as above. Panel D is a bar graph depicting lipid synthesis from acetate in HepG2 or (Panel E) 293T cells with stable PHD3 knockdown by shRNA or non-targeting control (n=3). C75=fatty acid synthase inhibitor (20 μM). Panel F is a photograph of an immunoblot depicting hydroxylation was assessed in endogenous ACC1 versus ACC2 by immunoprecipitation with isoform-specific antibodies and immunoblotting with OH-Pro antibody. Panel G depicts ACC2 hydroxyproline residues detected by mass spectrometry following transient overexpression of ACC2 in 293T cells and immunoprecipitation with ACC antibody (SEQ ID NOS 64-66, respectively, in order of appearance). Diagram shows the location of OH-Pro residues in ACC2 domains. #=modified prolines. Xcorr=cross correlation score. BT=biotin transferase domain. BCCP=biotin carboxyl carrier protein. Panel H depicts hydroxylation of transiently overexpressed wild type ACC2 or proline to alanine point mutants. Overexpressed ACC2 was immunoprecipitated with ACC antibody. Hydroxylation was assessed by immunoblot with OH-Pro antibody. Panel I depicts in vitro reconstituted hydroxylation assay with ACC2 peptides and recombinant PHD3 (n=2). Panel J depicts palmitate oxidation in complete media in 293T cells transiently overexpressing wild type ACC2 or ACC2 lacking the P450 hydroxylation site (n=3). Western blots show levels of overexpressed ACC2. **<0.01. Error bars indicate SEM.

FIG. 3 includes eight panels (Panels A-H), which show that PHD3 and the ACC2 hydroxylation site P450 promote ACC2 activity and ATP binding. Panel A depicts conservation of P450 in the ATP grasp domain. Alignment shows the ACC2 isoform in human (SEQ ID NO: 67), rat (SEQ ID NO: 68) and mouse (SEQ ID NO: 69), and ACC in C. elegans (SEQ ID NO: 70), drosophila (SEQ ID NO: 71) and S. cerevisiae (SEQ ID NO: 72), organisms lacking distinct ACC1/2 isoforms. Panel B, ACC activity was measured in 293T cell lysates overexpressing vector, wild type ACC2 (WT) or P450A mutant (n=3). Reactions were done ±the ACC allosteric activator citrate (2 mM). Western blots show overrexpressed ACC2 (Panel C), ACC activity in 293T cell lysates co-overexpressing vector, ACC2 or P450A along with either HA-PHD3 or empty vector (n=4). Reactions were done with citrate. Western blots show overexpressed ACC2 and HA-PHD3. Panel D, Model of the effect of PHD3 on FAO via ACC2 hydroxylation. Panel E, Molecular modeling to evaluate the location of P450 in the human ACC2 ATP-grasp domain relative to ATP and known nucleotide binding residues. Panel F, ATP-affinity of endogenous ACC2 from 293T cells stably expressing shRNA against PHD3 or non-targeting control. ATP-bound proteins were immunoprecipitated using ATP-affinity resin. Levels of immunoprecipitated ACC2 were analyzed by immunoblot with ACC2 antibody. Panel G, ATP-affinity of wild type and P450A ACC2 from transiently transfected 293T cells, as assessed by immunoprecipitation with ATP-affinity resin and immunoblot with ACC antibody. Panel H, ACC activity in 293T cell lysates overexpressing 2 μg ACC2 plasmid with PHD3 knockdown or control (n=3). Reactions were done with citrate. Western blots show loading controls. Knockdown was performed with shPHD3 #2. *<0.05, **<0.01, ***<0.001. Error bars indicate SEM.

FIG. 4 includes 13 panels (Panels A-M), which show that low PHD3 expression in AML correlates with greater sensitivity to treatment with FAO inhibitors. Panel A, Gene expression of PHD3 in patient samples across cancer types. Data obtained from the Ramaswamy multi-type cancer analysis on Oncomine. Panels B and C, Relative PHD3 gene expression in normal marrow versus AML patient samples. Data obtained from Valk and Andersson Leukemia Oncomine datasets. Panel D, PHD3 gene expression in leukemia cells. K562=CML cell line (black bar). MOLM14, KG1 and THP1=AML cell lines. Panel E, Palmitate oxidation by leukemia cell lines in complete RPMI media (n=3). Panel F, Viability of leukemia cells assessed by PI staining after 96 hr treatment with 0, 100, 200, 350 or 500 μM ranolazine (n=3). Panel G, Plot of data shown in (f) highlighting sensitivity to 500 μM ranolazine. Panel H, Viability of leukemia cells after 96 hr treatment with 0, 50, 100, 150 or 200 μM etomoxir (n=3). Panel I, Plot of data shown in (h) highlighting sensitivity to 150 μM etomoxir. Panel J, Viability of high PHD3 CML cell line (K562) compared to low PHD3 CML cell line (KU812) and low PHD3 AML cell lines (NB4) following 96 hr treatment with etomoxir (n=3). Panel K, Relative PHD3 gene expression in K562, KU812 and NB4 leukemia cell lines. ND=not detectable. Panel L, Endogenous ACC2 hydroxylation was measured in leukemia cell lines. ACC2 was immunoprecipitated with ACC2 antibody, and hydroxyproline was assessed by immunoblot with OH-Pro antibody. Because the ACC2 antibody cannot detect endogneous levels of ACC2 in whole cell lysates, an ACC antibody was used instead to show input. Panel M, ATP-affinity of endogenous ACC in leukemia cell lines, as assessed by immunoprecipitation with ATP-affinity resin and immunoblot with ACC antibody. **<0.01, ***<0.001. Error bars indicate SEM.

FIG. 5 includes five panels, A-E, which show the effects of PHD3 gene expression on fatty acid oxidation. Panel A is a photograph of a western blot showing knockdown of PHD3 gene expression in HepG2 cells. Panel B is a bar graph showing palmitate oxidation in HepG2 cells with PHD3 knockdown (n=3). Panel C is a bar graph showing palmitate oxidation in 786-O VHL−/− cells with constitutively stabilized HIF. Cells were transiently transfected with Dharmacon siGENOME SMARTpool EGLN3 siRNA (siPHD3) or Non-Targeting siRNA Pool #2 (siControl), and FAO was assessed 48 hr later (n=3). Panel D is a bar graph showing palmitate oxidation in 293T cells following 12 hr pre-incubation in normoxia or hypoxia (1% O). For 2 hr FAO analysis, cells were again maintained under normoxia or hypoxia (n=4). Panel E is a bar graph showing the effect of PHD3 levels on palmitate oxidation in complete media in ARNT-deficient cells, which have constitutively inactive HIF. FAO was assessed following transfection with human HA-PHD3 or vector alone.

FIG. 6, which includes two panels, A and B, provides representative mass spectra identifying the hydroxylated and non-hydroxylated versions of residue P450 in ACC2 peptides (SEQ ID NOS 73 and 73, respectively, in order of appearance). OH-Pro sites were identified by the expected +15.9949 molecular weight shift. ‘b’ fragments contain the N-terminal amino acid of the peptide and are labeled from the amino to the carboxyl terminus. ‘y’ fragments contain the C-terminal amino acid of the peptide are labeled from the carboxyl to the amino terminus.

FIG. 7 includes two panels, A and B, and depicts PHD3 repression of long chain fatty acid oxidation. Palmitate oxidation in complete media in 293T cells transiently overexpressing wild type ACC2 or ACC2 lacking the P450 hydroxylation sites (n=3). Western blots show levels of overexpressed ACC2 and/or variants.

FIG. 8 depicts the ATP affinity of wild type and P450G ACC2 point mutant from transiently transfected 293T cells, as assessed by immunoprecipitation with ATP-affinity resin and immunoblot with ACC antibody.

FIG. 9 depicts the structure of hydroxyproline.

FIG. 10 includes eight panels, A-H, and depicts that PHD3 represses fatty acid catabolism in response to nutrient abundance and in a manner independent of HIF and AMPK. Panel A is a bar graph showing palmitate oxidation in WT versus AMPKα KO MEFs expressing shRNA against PHD3 or non-silencing control (n=3). Panel B is a photograph of an immunoblot showing the impact of nutrient status on ACC hydroxylation. ACC hydroxylation in 293T cells was assessed following 12 h incubation in high versus low nutrient medium. High nutrient DMEM contains 4.5 g/L glucose and serum. Low nutrient DMEM contains 1 g/L glucose without serum. ACC was immunoprecipitated and hydroxylation was detected by immunoblot. With endogenous PHD3 (vector lanes), ACC is hydroxylated to a greater extent under a nutrient replete versus nutrient deprived state. Transient PHD3 overexpression enables hydroxylation under low nutrient conditions. Panel C is a photograph of an immunoblot showing 293T cells stably expressing shRNA against PHD3 or non-targeting control were incubated 12 h in high or low nutrient media prior to analyzing ACC hydroxylation by IP and immunoblot. Panel D is a photograph of an immunoblot showing ACC hydroxylation dynamically responds to cellular nutrient cues. WT immortalized MEFs were incubated in high (4.5 g/L glucose DMEM with serum) or low (1 g/L glucose DMEM without serum) nutrient medium for 6 h, or in low nutrient medium for 6 h followed by adding back high nutrient medium for 5 or 10 min. ACC was immunoprecipitated in lysis buffer containing the PHD inhibitor DMOG (1 mM) to minimize further hydroxylation in the lysis buffer. Hydroxylation was detected by immunoblot. Panel E is a bar graph showing the impact of PHD3 knockdown on the ability of immortalized MEFs to modulate palmitate oxidation levels in response to low or high nutrient medium (n=3). Panel F is a bar graph showing the impact of PHD3 knockdown on the ability of 293T cells to suppress FAO in response to supplementing low glucose, serum-free medium with dimethyl ketoglutarate (+kg, 5 mM) for 6 h prior to FAO analysis. Dimethyl ketoglutarate was also maintained in the medium during 2 h FAO analysis (n=3). Panel G is a bar graph showing short, medium and long chain acylcarnitine levels as measured by metabolomics analysis of 293T cells grown in high nutrient medium following stable knockdown with shRNA against PHD3 or control. Levels were normalized to cell count in parallel plates (n=6 for control, n=3 for shPHD3). Panel H is a schematic showing a two-part model of the bioenergetic-versus nutrient-sensitive modes of ACC2 regulation. Under low nutrient conditions, AMPK responds to the AMP/ATP ratio to phosphorylate and inhibit ACC2, thus promoting long chain fatty acid mitochondrial import and oxidation. Under high nutrient conditions, PHD3 hydroxylates and activates ACC2 to limit long chain FAO. *p<0.05, **p<0.01, ***p<0.001. Data represent mean±SEM.

FIG. 11 includes six panels, A-F, showing that PHD3 expression is repressed in AML, contributing to altered ACC and a dependency on FAO that can be pharmacologically targeted. Panel A, PHD3 gene expression across AML patient samples analyzed from datasets in The Cancer Genome Atlas (TCGA). Patients were classified as low PHD3 vs. high PHD3 based on performing univariate clustering on PHD3 expression levels using a Gaussian mixture model with two clusters (low and high). Panel B, Box plot showing stratification of low and high (PHD3 gene expression in TCGA AML patient samples, as calculated in (D). Nearly 80% of patients fell into the low PHD3 group. Panel C, Table of top curated gene set collections that are inversely correlated with the high-PHD3 cluster of AML patient samples, as determined by gene set enrichment analysis. Pathways were ranked by false discovery rate (FDR) q value and normalized enrichment score (NES). Panel D, qPCR analysis of PHD3 gene expression in leukemia cells using PPIA as a reference gene. K562=CML cell line (black bar). MOLM14, KG1, THP1, NB4 and U937=AML cell lines. Panel E, PHD3 gene expression in K562 cells stably expressing shRNA against PHD3 or non-silencing control (n=3). Panel F, Stable PHD3 knockdown boosts palmitate oxidation in K562 CML cells (n=3).

FIG. 12 includes sixteen panels, A-P, showing PHD3 overexpression in low-PHD3 AML cells limits FAO and decreases cell proliferation and colony formation. Panel A, Palmitate oxidation in MOLM14 and THP1 cells following stable overexpression of empty vector or PHD3 (n=3). Immunoblots show stable overexpression of HA-PHD3. Panel B, Growth curves of MOLM14 and THP1 cells stably overexpressing vector or PHD3 (n=3). Panel C, ATP CellTiter-Glo analysis in MOLM14 and THP1 cells stably overexpressing vector or PHD3 (n=4). Panel D, Colony formation assay with MOLM14 and THP1 cells stably overexpressing vector or PHD3. Colony forming units (CFU) were counted 8 days after plating MOLM14 and 20 days after plating THP1 (n=3). Panel E, Representative images from colony formation assays with MOLM14 or THP1 cells stably overexpressing vector or PHD3. MOLM14 colonies were imaged on day 8 and THP1 colonies imaged on day 20 using an inverted microscope (Nikon Eclipse Ti-U) at 200× magnification and SPOT camera software 5.0. Panel F, Immunoblot showing HA-PHD3 overexpression in K562 cells. Panels G and H, Colony formation assay and representative images from K562 cells stably expressing PHD3 or vector. CFU were counted and imaged 10 days after plating (n=3). Panel I, ACC inhibition restores cell growth following PHD3 overexpression in MOLM14 cells. Cells were treated with S2E (50 μM, Sigma), metformin (1 mM, Sigma) or vehicle following viral infection with PHD3 and throughout the duration of the experiment. Infected cells were selected with puromycin. Following FACS to collect PI-negative cells, 8000 cells were plated per well of a 96-well plate, and cell number at 72 h was determined by cell counting (n=3). Panel J, Metformin partially blocks the growth inhibitory effects of PHD3-overexpression in MOLM14 cells, as measured in soft agar assays. Colony formation was assessed in MOLM14 cells stably overexpressing vector or PHD3 in the presence or absence of metformin (Met, 1 mM). CFU were counted 8 days after plating (n=2). Panel K, qPCR analysis of PHD3 gene expression in primary human CD34+ cells from bone marrow filtrate of a healthy donor or AML patient samples (690a, 2093 and 2266). PPIA was used as a reference gene. Panel L, ATP CellTiter-Glo analysis of cell viability in AML patient samples following stable overexpression of empty vector or PHD3 (n=4). Panel M, qPCR analysis of PHD3 gene expression in primary mouse CD11b control cells or AML cells obtained from Hoxa9 Meis1 and MLL-AF9 mouse models. Panel N, PHD3 gene expression in primary mouse MLL-AF9 AML cells following stable overexpression of empty vector or PHD3 (n=2). Panel O, Colony formation assay with MLL-AF9 AML cells stably overexpressing vector or PHD3. Colonies were counted 10 days after plating (n=3). Panel P, Kaplan-Meier survival curves of NSG mice xenotransplanted with MOLM14 human AML cell line stably overexpressing vector or PHD3 (n=5). *p<0.05, **p<0.01, ***p<0.001. Data represent mean±SEM.

FIG. 13 includes twelve panels, A-L, showing PHD3 represses long chain FAO under nutrient-replete conditions. Panels A and B, Palmitate oxidation and ACC2 gene expression in 293T cells stably expressing shRNA against ACC2 or non-targeting control (n=3). Panel C, Immunoblot showing that ACC2 is present in both bands detected with the ACC antibody. Following stable knockdown of ACC2 in 293T cells, both the upper and lower bands are decreased upon blotting with antibodies against total ACC or ACC2. ACC1 and tubulin levels are shown as controls. Panel D, Validation of HIF deficiency in HIFβ-null mouse hepatoma cells. PHD3 gene expression and HIF target gene expression in HIFβ-deficient cells transiently transfected with siRNA against PHD3 or control, and also treated with or without the HIFα-stabilizing compound CoCl₂ (250 μM, 6 h) (n=4). Panel E, Immunoblot analysis of phospho-ACC, total ACC and AMPKα protein levels in wild type and AMPKα knockout MEFs in response to AICAR stimulation. MEFs were cultured in serum-free DMEM overnight and treated with or without AICAR (1 mM for 1 h, Cell Signaling Technology). Panel F, PHD3 gene expression in WT and AMPKα KO MEFs following stable expression of shRNA against PHD3 or non-silencing control (n=4). Panel G, Palmitate oxidation in AMPKα KO MEFs overexpressing PHD3 or vector (n=3). Western blot shows HA-PHD3 overexpression. Panel H, ACC can be phosphorylated by AMPK in a nutrient-sensitive manner independently of PHD3. Whole cell lysates were collected from stable shPHD3 or control MEFs which had been incubated in high or low nutrient medium for 6 h, or low nutrient medium for 6 h followed by 10 min of adding back high nutrient medium. Samples were analyzed by immunoblot for phospho-ACC, total ACC and tubulin. Panel I, PHD3 gene expression in WT MEFs following stable knockdown of PHD3 or non-silencing control. Panels J and K, ACC activity under different nutrient conditions in WT MEF lysates stably co-expressing ACC2 along with shRNA against PHD3 or control (n=3). Lysates were collected from MEFs that had been incubated 6 h in low nutrient medium, or 6 h in low nutrient medium followed by 10 min restoration of high nutrients. Panel L, Impact of PHD3 knockdown on the ability of 293T cells to modulate palmitate oxidation levels in response to low or high nutrient medium (n=3). *p<0.05, **p<0.01, ***p<0.001. Data represent mean±SEM.

FIG. 14 includes thirteen panels, A-M, showing links between Low PHD3 expression in AML and high oxidative metabolism. Panels A-G are a series of box plots showing expression of oxidative and bioenergetic gene sets or of the individual genes ACC2, LKB1 or AMPKα2 in low-PHD3 versus high-PHD3 AML patient samples available on TCGA. Gene sets were obtained from the Broad Institute's Molecular Signatures Database (MSigDB). FDR=false discovery rate. Panels H-I, PHD1 and PHD2 gene expression in K562 CML cells (black bar) and a panel of AML cell lines. In AML, neither PHD is silenced to the same extent as PHD3. Panel J, Viability of high-PHD3 CML cell line (K562) compared to low-PHD3 AML cell line (NB4) or low-PHD3 CML cell line (KU812) following 96 h treatment with indicated doses of etomoxir (n=3). Asterisks show significance compared to K562 cell response. Panel K, Viability of the high-PHD3 CML cell lines MEG01 and K562 compared to the low-PHD3 AML cell line (NB4) following 96 h treatment with indicated doses of ranolazine (n=3). MEG01 is less sensitive to a high dose of ranolazine compared to the low-PHD3 cells. Panel L, PHD3 gene expression in CML cell lines relative to K562. Panel M, In K562 CML cells that normally express high PHD3, stable PHD3 knockdown does not create the dependency on fatty acid catabolism that is observed in AML. K562 cells expressing shPHD3 or shControl were treated 96 h+/−ranolazine at the indicated doses, and viability was assessed by PI staining (n=3). *p<0.05, **p<0.01, ***p<0.001. Bar graphs and cell viability curves represent mean±SEM.

FIG. 15 includes 10 panels, A-J, showing PHD3 modulation in leukemia cell lines. Panel A, PHD3 expression in MOLM14 and THP1 cells following stable overexpression of vector or PHD3 (n=3). Panel B, Palmitate oxidation in HepG2 cells treated with etomoxir during the 2 h FAO assay (100 μM, n=3). Panels C and D, PHD3 expression and growth curves in K562 cells following stable overexpression of PHD3 or vector (n=3). Panel E, Growth curves of K562 cells stably expressing shRNA against PHD3 or control (n=3). Panel F, Colony formation assay with K562 cells stably expressing shRNA against PHD3 or non-silencing control. Colony forming units (CFU) were counted 10 days after plating (n=2). Panel G, Representative images from colony formation assays with K562 cells stably expressing shRNA against PHD3 or non-silencing control. Colonies were imaged on day 10 using an inverted microscope (Nikon Eclipse Ti-U) at 200× magnification and SPOT camera software 5.0. Panel H, The ACC inhibitor S2E increases palmitate oxidation. MOLM14 cells were incubated with S2E (50 μM) or vehicle for 3 days. FAO was measured following 3 h incubation with radiolabeled palmitate in the presence of S2E or vehicle. Panel I, PHD3 expression in MOLM14 cells following stable overexpression of vector or PHD3 (n=3). Cells with this lower level of PHD3 overexpression were used for the colony formation assay shown in FIG. 14, Panel I. Panel J, Representative images from colony formation assays with MOLM14 cells stably overexpressing vector or PHD3 in the presence of metformin (1 mM) or vehicle (n=2). Arrowheads in the lower panels indicate colonies. **p<0.01, ***p<0.001. ns=non-significant. Data represent mean±SEM.

FIG. 16 includes three panels, A-C, showing the sorting of live cells by FACS. Panels A-C, are a series of FACS plots showing gating for propidium iodide-negative MOLM14, THP1 and K562 cells with stable overexpression or knockdown of PHD3 or control.

### DETAILED DESCRIPTION

The present disclosure provides, among other things, compositions and methods useful for treating and diagnosing a number of conditions including, but not limited to, cancer, cardiovascular disease, obesity, and metabolic disorders. While in no way intended to be limiting, exemplary compositions, kits, and applications are elaborated on below.

### Polypeptides

The disclosure features polypeptides comprising a portion of ACC2 (e.g., any isoform from any species expressing an ACC2 polypeptide) containing the proline residue at positions 343, 450, and/or 2131 (relative to SEQ ID NO:2). An exemplary amino acid sequence for human ACC2 (isoform 1) is as follows (SEQ ID NO: 2):

| | |
|---|---|
| 1 | mvlllclscl ifscltfswl kiwgkmtdsk pitksksean lipsqepfpa sdnsgetpqr |
| 61 | ngeghtlpkt psqaepashk gpkdagrrrn slppshqkpp rnplsssdaa pspelqangt |
| 121 | gtqgleatdt nglsssarpq gqqagspske dkkganikrq lmtnfilgsf ddyssdedsv |
| 181 | agssrestrk gsraslgals leaylttgea etrvptmrps msglhlvkrg rehkkldlhr |
| 241 | dftvaspaef vtrfggdrvi ekvlianngi aavkcmrsir rwayemfrne rairfvvmvt |
| 301 | pedlkanaey ikmadhyvpv pggpnnnnya nvelivdiak ripvqavwag wghasenpkl |
| 361 | pellckngva flgppseamw algdkiastv vaqtlqvptl pwsgsgltve wteddlqqgk |
| 421 | risvpedvyd kgcvkdvdeg leaaerigfp lmikaseggg gkgirkaesa edfpilfrqv |
| 481 | qseipgspif lmklaqharh levqiladqy gnavslfgrd csiqrrhqki veeapatiap |
| 541 | laifefmeqc airlaktvgy vsagtveyly sqdgsfhfle lnprlqvehp ctemiadvnl |
| 601 | paaqlqiamg vplhrlkdir llygespwgv tpisfetpsn pplarghvia aritsenpde |
| 661 | gfkpssgtvq elnfrssknv wgyfsvaatg glhefadsqf ghcfswgenr eeaisnmvva |
| 721 | lkelsirgdf rttveylinl letesfqnnd idtgwldyli aekvqaekpd imlgvvcgal |
| 781 | nvadamfrtc mtdflhsler gqvlpadsll nlvdveliyg gvkyilkvar qsltmfvlim |
| 841 | ngchieidah rlndggllls yngnsyttym keevdsyrit ignktcvfek endptvlrsp |
| 901 | sagkltqytv edgghveags syaemevmkm imtlnvqerg rvkyikrpga vleagcvvar |
| 961 | lelddpskvh paepftgelp aqqtlpilge klhqvfhsvl enitnvmsgf clpepvfsik |
| 1021 | lkewvqklmm tlrhpslpll elqeimtsva gripapveks vrrvmaqyas nitsvlcqfp |
| 1081 | sqqiatildc haatlqrkad revffintqs ivqlvqryrs girgymktvv ldllrrylrv |
| 1141 | ehhfqqahyd kcvinlreqf kpdmsqvldc ifshaqvakk nqlvimlide lcgpdpslsd |
| 1201 | elisilnelt qlsksehckv alrarqilia shlpsyelrh nqvesiflsa idmyghqfcp |
| 1261 | enlkklilse ttifdvlptf fyhankvvcm aslevyvrrg yiayelnslq hrqlpdgtcv |
| 1321 | vefqfmlpss hpnrmtvpis itnpdllrhs telfmdsgfs plcqrmgamv afrrfedftr |
| 1381 | nfdeviscfa nvpkdtplfs eartslysed dckslreepi hilnvsiqca dhledealvp |
| 1441 | ilrtfvqskk nilvdyglrr itfliaqeke fpkfftfrar defaedriyr hlepalafql |
| 1501 | elnrmrnfdl tavpcanhkm hlylgaakvk egvevtdhrf firaiirhsd litkeasfey |
| 1561 | lqnegerlll eamdelevaf nntsvrtdcn hiflnfvptv imdpfkiees vrymvmrygs |
| 1621 | rlwklrvlqa evkinirqtt tgsavpirlf itnesgyyld islykevtds rsgnimfhsf |
| 1681 | gnkqgpqhgm lintpyvtkd llqakrfqaq tlgttyiydf pemfrqalfk lwgspdkypk |
| 1741 | diltytelvl dsqgqlvemn rlpggnevgm vafkmrfktq eypegrdviv ignditfrig |
| 1801 | sfgpgedlly lrasemarae gipkiyvaan sgarigmaee ikhmfhvawv dpedphkgfk |
| 1861 | ylyltpqdyt risslnsvhc khieeggesr ymitdiigkd dglgvenlrg sgmiagessl |
| 1921 | ayeeivtisl vtcraigiga ylvrlgqrvi qvenshiilt gasalnkvlg revytsnnql |
| 1981 | ggvqimhyng vshitvpddf egvytilewl sympkdnhsp vpiitptdpi dreieflpsr |
| 2041 | apydprwmla grphptlkgt wqsgffdhgs fkeimapwaq tvvtgrarlg gipvgviave |
| 2101 | trtvevavpa dpanldseak iiqqagqvwf pdsayktaqa vkdfnreklp lmifanwrgf |
| 2161 | sggmkdmydq vlkfgayivd glrqykqpil iyippyaelr ggswvvidat inplciemya |
| 2221 | dkesrggvle pegtveikfr kkdliksmrr idpaykklme qlgepdlsdk drkdlegrlk |
| 2281 | aredlllpiy hqvavqfadf hdtpgrmlek gvisdilewk tartflywrl rrllledqvk |
| 2341 | qeilqasgel shvhiqsmlr rwfvetegav kaylwdnnqv vvqwleqhwq agdgprstir |
| 2401 | enitylkhds vlktirglve enpevavdcv iylsqhispa eraqvvhlls tmdspast |

Proline residue 450 is emphasized in bold and underlining. One of skill in the artisan would appreciate that the exact position of amino acid residues in a given polypeptide varies from species to species and with truncations or extension of the naturally-occurring sequence. The artisan would therefore appreciate that references herein to a polypeptide (or a fragment thereof) comprising an amino acid substitution at position 450 relative to SEQ ID NO:2, include, e.g., an amino acid substitution at position 440 of SEQ ID NO:3 (murine ACC2):

| | |
|---|---|
| 1 | mvlllfltcl vfscltfswl kiwgkmtdsk pltnskvean llsseeslsa selsgeqlqe |
| 61 | hgdhsclsyr gprdasqqrn slpsscqrpp rnplssndtw pspelqtnwt aapgpevpda |
| 121 | nglsfparpp sqrtvspsre drkqahikrq lmtsfilgsl ddnssdedps agsfqnssrk |
| 181 | ssraslgtls qeaalntsdp eshaptmrps msglhlvkrg rehkkldlhr dftvaspaef |
| 241 | vtrfggnrvi ekvlianngi aavkcmrsir rwayemfrne rairfvvmvt pedlkanaey |
| 301 | ikmadqyvpv pggpnnnnya nveliidiak ripvqavwag wghasenpkl pellckheia |
| 361 | flgppseamw algdkiasti vaqtlqiptl pwsgsgltve wtedsrhqgk cisvpedvye |
| 421 | qgcvkdvdeg lqaaekigfp lmikaseggg gkgirkaesa edfpmlfrqv qseipgspif |
| 481 | lmklaqnarh levqvladqy gnayslfgrd csiqrrhqki ieeapatiaa pavfefmeqc |
| 541 | avllakmvgy vsagtveyly sqdgsfhfle lnprlqvehp ctemiadvnl paaglqiamg |
| 601 | vplhrlkdir llygespwgv tpipfetpls ppiarghvia aritsenpde gfkpssgtvq |
| 661 | elnfrsnknv wgyfsvaaag glhefadsqf ghcfswgenr eeaisnmvva lkelsirgdf |
| 721 | rttveylvnl letesfqnnd idtgwldhli aqrvqaekpd imlgvvcgal nvadamfrtc |
| 781 | mteflhsler gqvlpadsll nivdveliyg gikyalkvar qsltmfvlim ngchieidah |
| 841 | rlndggllls yngssyttym keevdsyrit ignktcvfek endptvlrsp sagklmqytv |
| 901 | edgdhveags syaemevmkm imtlnvqesg rvkyikrpgv ileagcvvar lelddpskvh |
| 961 | aaqpftgelp aqqtlpilge klhqvfhgvl enitnvmsgy clpepffsmk lkdwvqklmm |
| 1021 | tlrhpslpll elqeimtsva gripapveka vrrvmaqyas nitsvlcqfp sqqiatildc |
| 1081 | haatlqrkad revffmntqs ivqlvqryrs gtrgymkavv ldllrkylnv ehhfqqahyd |
| 1141 | kcvinlreqf kpdmtqvldc ifshsqvakk nqlvtmlide lcgpdptlsd eltsilcelt |
| 1201 | qlsrsehckv alrarqvlia shlpsyelrh nqvesiflsa idmyghqfcp enlkklilse |
| 1261 | ttifdvlptf fyhenkvvcm aslevyvrrg yiayelnslq hrelpdgtcv vefqfmlpss |
| 1321 | hpnrmavpis vsnpdllrhs telfmdsgfs plcqrmgamv afrrfeeftr nfdeviscfa |
| 1381 | nvqtdtllfs kactslysee dskslreepi hilnvaiqca dhmedealvp vfrafvqskk |
| 1441 | hilvdyglrr itflvaqere fpkfftfrar defaedriyr hlepalafql elsrmrnfdl |
| 1501 | tavpcanhkm hlylgaakvk eglevtdhrf firaiirhsd litkeasfey lgnegerlll |
| 1561 | eamdelevaf nntsvrtdcn hiflnfvptv imdplkiees vrdmvmrygs rlwklrvlqa |
| 1621 | evkinirqtt sdsaipirlf itnesgyyld islyrevtds rsgnimfhsf gnkqgslhgm |
| 1681 | lintpyvtkd llqakrfqaq slgttyvydf pemfrqalfk lwgspekypk diltytelvl |
| 1741 | dsqgqlvemn rlpgcnevgm vafkmrfktp eypegrdavv ignditfqig sfgigedfly |
| 1801 | lrasemarte gipqiylaan sgarmglaee ikqifqvawv dpedphkgfr ylyltpqdyt |
| 1861 | qissqnsvhc khiedegesr yvivdvigkd anlgvenlrg sgmiageasl ayektvtism |
| 1921 | vtcralgiga ylvrlgqrvi qvenshiilt gagalnkvlg revytsnnql ggvqimhtng |
| 1981 | vshvtvpddf egvctilewl sfipkdnrsp vpittpsdpi dreieftptk apydprwmla |
| 2041 | grphptlkgt wqsgffdhgs fkeimapwaq tvvtgrarlg gipvgviave trtvevavpa |
| 2101 | dpanldseak iiqqagqvwf pdsayktaqv irdfnkerlp lmifanwrgf sggmkdmyeq |
| 2161 | mlkfgayivd glrlyeqpil iyippcaelr ggswvvldst inplciemya dkesrggvle |
| 2221 | pegtveikfr kkdlvktirr idpvckklvg qlgkaqlpdk drkelegqlk areelllpiy |
| 2281 | hqvavqfadl hdtpghmlek giisdvlewk tartffywrl rrllleaqvk qeilraspel |
| 2341 | nhehtqsmlr rwfvetegav kaylwdsnqv vvqwleqhws akdglrstir eninylkrds |
| 2401 | vlktiqslvq ehpevimdcv aylsqhltpa eriqvaqlls ttespass |

Proline residue 440 is emphasized in bold and underlining. Likewise, one of skill in the art would recognize that references herein to a polypeptide (or a fragment thereof) comprising an amino acid substitution at position 450 relative to SEQ ID NO:2, include, e.g., an amino acid substitution at position 446 of SEQ ID NO:4 (rat ACC2):

| | |
|---|---|
| 1 | mvlllfltyl vfscltiswl kiwgkmtdsr plsnskvdas llpskeesfa sdqseehgdc |
| 61 | scplttpdqe elashggpvd asqqrnsvpt shqkpprnpl ssndtcsspe lqtngvaapg |
| 121 | sevpeanglp fparpqtqrt gsptredkkq apikrqlmts filgslddns sdedpssnsf |
| 181 | qtssrkgsrd slgtcsqeaa lntadpesht ptmrpsmsgl hlvkrgrehk kldlhrdftv |
| 241 | aspaefvtrf ggnrvietvl ianngiaavk wmrsirrway emfrnerair fvvmvtpedl |
| 301 | kanaeyykma dpvlpvpggp nnnnyanvel iidiakripv qavwagwgha senpklpell |
| 361 | ckhgiaflgp rvrpmlglgd rlsstivaqt lqiptlpwsg sgltvewted sqhqgkcisv |
| 421 | tedvyeqgcv rdvdeglqaa ekvgfplmik aseggggkgi rqaesaedfp cffrqvqsei |
| 481 | pgspiflmkl aqnarhlevq vladqygnav slfgrdcsiq rrhqkiieea paniaapavf |
| 541 | efmeqcavll aktvvyvsag tvgylysqdg sfhflelnpr lqvehpctem iadvnlpaaq |
| 601 | lqiamgvplh rlkdirllyg espwgvtpvs fetplsppia rghviaarit senpdeafkp |
| 661 | ssgtvqelnf rsnknvwgyf svaaagglhe fpisqfghcf swgenqeeai snmvvalkel |
| 721 | sirgdfrttv eylvnllete slqnndidtg wldhliaqrv qaekpdimlg vvfgalnvad |
| 781 | amfrtcitef lhslergqvl padsllnivd veliyggiky vlkvarqslt mfvlimngch |
| 841 | ieidahrpnd gglllsyngs syttymkeev dsyritignk tcvfekendp tvlrspsagk |
| 901 | lmqytvedgq hvevgssyae mevmkmimtl nvqesgrvny ikrpgavlea gcvvakleld |
| 961 | dpskvhaaqp ftgelpaqqt lpilgerlhq vfhsvlenlt nvmngyclpe pffsmklkdw |
| 1021 | vekpmmtlrh pslpllelqe imtsvadrip vpvekavrrv faqdasnits vlcqfpsqqi |
| 1081 | atildchaat lqrkvdreaf fmntqsivql iqryrsgtrg imkavvldll rrylnvehhf |
| 1141 | qqahydkcvi nlreqfkadm trvldcifsh sqvakknqlv tmlidelcgp dptlseelts |
| 1201 | ilkeltqlsr sehckvalra rqvliashlp syelrhnqve ssscqpltcn ghqfcpenlk |
| 1261 | klilsettif dvlptffyha nkvvcmasle vyvrrgyiay elnslqhrel pdgtcvvefq |
| 1321 | fmlpsshpnr mampinvsdp dllrhskelf mdsgfsplch qrmgamvafr rfeeftrnfd |
| 1381 | eviscfanvp tdtplfskac tslyseedsk slqeepihil nvaiqcadhm ederlvpvfr |
| 1441 | afvqskkhil vdyglrritf liaqekefpk fftfrardef aedriyrhle pglafqlels |
| 1501 | rmrnfdltav pcanhkmhly lgaakvkegl evtdhrffir aiirhsdlit keasfeylqn |
| 1561 | egerllleam delevafnnt svrtdcnhif lnfvahvimd plkieesvra mvmrygsrlw |
| 1621 | klrvlqaqvk inirqttsdc avpirlfitn esgyyldisl ykevtdsrsg nimfhsfgnk |
| 1681 | qgslhgmlin tpyvtkdllq akrfqaqslg ttyvydfpem frqalfklwg spekygpdil |
| 1741 | tytelvldsq gqlvemnrlp gcnevgmvvf kmrfktpeyp egrdtivign ditfqigsfg |
| 1801 | igedflylra semartegip qiylaansga vlglseeikg ifqvawvdpe dpykgfryly |
| 1861 | lyltpqdytq issqnsvhck hiedegesgi ivdvigkdss lgvenlrgsg miageaslay |
| 1921 | eknvtismvd craigigayl vrlgqrviqv enshiiltga galnkvlgre vytsnnqlgg |
| 1981 | vqimhtngvs hvtvpddfeg vctilewlsy ipkdngspvp iitpsdpidr eieftptkap |
| 2041 | ydprwllagr phptlkgtwq sgffdhgsfk eimapwdqtv vtgrarlggi pvgviavetr |
| 2101 | svevavpahp anldseakii qqagqvwfpd safktaqvir dfnqehlllm ifanwrgfsg |
| 2161 | gmkdmseqml kfgayivdsl rlskqpvliy ippgaelrgg swvvldssin plciemyadk |
| 2221 | esrggvlepe gtveikfrkk dlvktirrid pvckkllepa gdtqlpdkdr kelesqlkar |
| 2281 | edlllpiyhq vavqfadlhd tpghmlkkgi isdvlewktt rtyfywrlrr llleaqykqe |
| 2341 | ilraspelsh ehtqsmlrrw fvetegavka ylwdsnqvvv qwleqhwsar dnlrstiren |
| 2401 | lnylkrdsvl ktiqslvqeh peatmglcgy lsghltpaeq mqvvqllstt espash |

Proline residue 446 is emphasized in bold and underlining. Likewise, one of skill in the art would recognize that references herein to a polypeptide (or a fragment thereof) comprising an amino acid substitution at position 450 relative to SEQ ID NO:2, include, e.g., an amino acid substitution at position 371 of SEQ ID NO:5 (Xenopus ACC2):

| | |
|---|---|
| 1 | megdkeqlpk ppiaeaetpa esddnllrtq aegttsgqiq dtnsgvnsgt lppraaslsk |
| 61 | peqkqlkfap srgtepvnpk prkqplskfi lgssednsdd defacgsfkt tkrnsqaslg |
| 121 | sqtpslsslp eteslptmrs smsglhlvkk grdhkkldlh rdftvasphe fvtrfggnrv |
| 181 | iekvlianng iaavkcmrsi rrwsyemfrn erairfvvmv tpedlkanae yikmadhyvp |
| 241 | vpggpnnnny anvelivdia kripvqavwa gwghasenpk lpellqkqni aflgppsqam |
| 301 | walgdkiast ivaqavgipt lswsgdglll elkpddkqqq niicvppevy ekgcvkdade |
| 361 | gleaaerigy pvmikasegg ggkgirmaer aedfpslfrq vqteapgspi fvmklaqhar |
| 421 | hlevqiladq yghayslfgr dcsiqrrhqk iieeapatva tpsvfeymeq cavrlakmvg |
| 481 | yvsagtveyl ysedgsfhfl elnprlqveh pctemicdvn lpaaqlqism gvplyrikdi |
| 541 | rvlygetpwg dspicfenpv napnprghvi aaritsenpd egfkpssgtv qelnfrsskn |
| 601 | vwgyfsvaaa gglhefadsq fghcfswgen reeaisnmvv alkelsirgd frttveylik |
| 661 | lletesfqnn eidtgwldhl iaekvqaekp dtmlgvvcga lnvadalfqt cmneflhcle |
| 721 | rgqvlpaasl lnivdvelis ervkyklkva rqslttyvii lnnshieidv hrlsdgglll |
| 781 | sydgnsytty mkeevdryri tignktcvfe kendptvlrs pstgkllqyt vedgshvnag |
| 841 | ecfaeievmk mvmaltvqep gqihyvkrpg avlesgcmva qidlddpskv lqaepytgsl |
| 901 | lpqqtlpiig eklhqvfhsv lenlinvmng yclpepyftv kikewvhklm ktlrdpslpl |
| 961 | lelqeimtsv stripptver sirkimaqya snitsvlcqf psqqiasild shaatlqrka |
| 1021 | drevffmntq sivqlvqryr sgirgymksv vldllrrylq vetqfqhshy dkcvihlreq |
| 1081 | ykpdmtpvle cifshaqvak knflvtmlid qlcgrdptlt delmailnel tqlsktehsk |
| 1141 | valrarqvli ashlpsyelr hnqvesifls aidlyghqfc pdnlkklils etsifdvlpn |
| 1201 | ffyhnnqvvr maalevyvrr gyiayelnsl qhhqlrdctc vvefqfmlps shpnreispt |
| 1261 | lsrmslpisa thleinrqss elfmdsgfsp lcqrmgvmva fnkfedftrn fdeviscfad |
| 1321 | ppldsplfse vrssfydeed nknireepih ilnvalksvd rmedeelvsv frtfcqskkn |
| 1381 | ilvdyglrri tfliaqqref pkfftfrard efaedriyrh lepalafqle lnrmrnfdln |
| 1441 | avpcanhkmh lylgaakvaa gievtdyrff vraiirhsdl itkeasfeyl qnegerllle |
| 1501 | amdelevafn npsvrtdcnh iflnfvptvi mdpskieesv rsmvmrygsr lwklrvlqae |
| 1561 | vkinirltpt gkaipirlfl tnesgyyldi slykevtdpa tgqimfhsyg dkhghmhgml |
| 1621 | intpyvtkdl lqskrfqaqs lgttyvydfp emfrqalfkl wrsgekypkd iltytelvld |
| 1681 | tqgqlvqlnr lpggnevgmv afkmnlktpe ypngreiivi cnditykigs fgpqedllfl |
| 1741 | ktselarkeg ipriyiaans gariglaeel rhmfqvawnn psdpykgfky lylrpqdytk |
| 1801 | issmnsahce hvedegesry vltdiigkee gigvenlrgs gtiagessla ykeivtigmv |
| 1861 | tcraigigay lvrlgqrviq venshiiltg asalnkvlgr evytsnnqlg gvqimcnngv |
| 1921 | shtmvpddfe gvytilqwls ympkdngspv pvippmdpvd rqiefmptka pydprwmlag |
| 1981 | rphptikgew qrgffdhgsf meimgrwaqt vvvgrarlgg ipvgviavet rsvemavpad |
| 2041 | panldseaki iqqagqvwfp dsafktaqai kdfnrerlpl lifanwrgfs ggmkdmydqv |
| 2101 | lkfgayivds lrefkqpvlv yippyaelrg gswvvidpti nplymelyad kdsrggvlep |
| 2161 | egtveirfrk kdliktmrri dpvytqiveq lgspeltege rkelekklrl reeqllpiyh |
| 2221 | qvavrfadlh dtpgrmqekg vitdilewkd arsflywrlr rllleemvks eilhansels |
| 2281 | dihiqsmlrr wfmetegavk tylwdnnqvv vewlekhlqe edearsaire nikylkkdya |
| 2341 | lkhirglvqa npevamdciv hmtqhitpaq raqltrllst mdntpps |

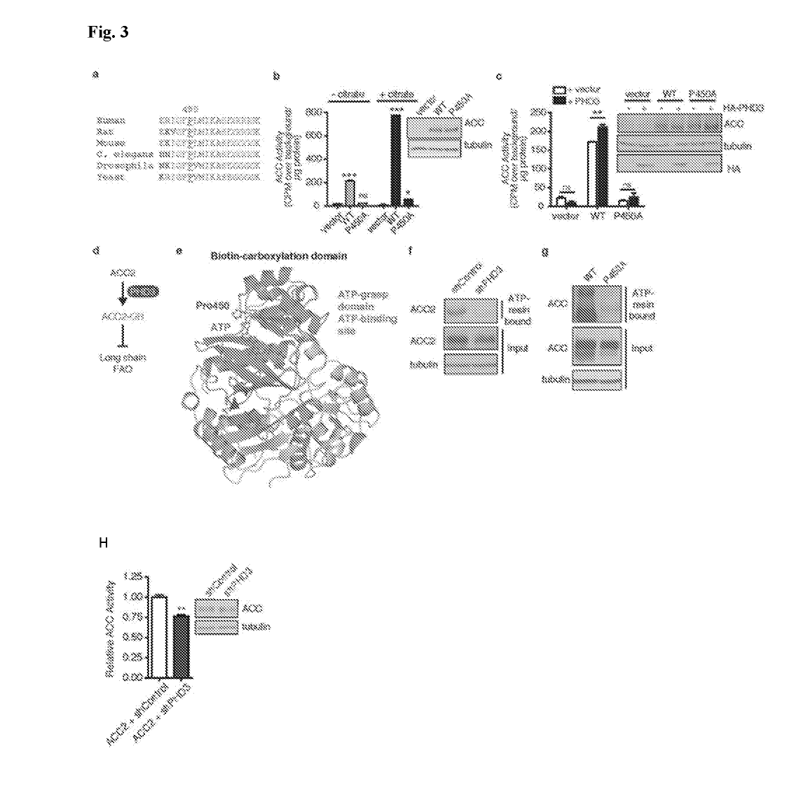

Proline residue 371 is emphasized in bold and underlining. Further examples of the relevant proline residue within the context of amino acid sequences from other species are set forth in FIG. 3, Panel A (e.g., C. elegans, Drosophila, and yeast sequences). It is well within the purview of the artisan to identify the corresponding proline residue in ACC2 amino acid sequences from other species, e.g., using publicly available software tools, such as Clustal W2 or BLAST.

In some embodiments, a polypeptide described herein comprises at least 8 (e.g., at least 9, 10, 11, 12, 13, 14, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1250, 1500, 1775, or 2000) consecutive amino acids of an ACC2 polypeptide (of any species), which consecutive amino acids include the proline residue at position 450 relative to SEQ ID NO:2, but the polypeptide does not comprise the entire amino acid sequence of ACC2.

In some embodiments a polypeptide described herein comprises at least 8 consecutive amino acids of an ACC2 polypeptide (of any species), which consecutive amino acids include the proline residue at position 450 relative to SEQ ID NO:2, but the polypeptide comprises no more than 2300 (e.g., no more than 2200, 2100, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, or 15) consecutive amino acids of ACC2.

In some embodiments a polypeptide described herein comprises at least 8 consecutive amino acids of an ACC2 polypeptide (of any species), which consecutive amino acids include the proline residue at position 450 relative to SEQ ID NO:2, but the polypeptide comprises no more than 98 (e.g., 95, 90, 85, 80, 75, 70, 65, 60, 55, 50, 45, 40, 35, 30, 25, 20 or 15) % of a full-length ACC2 polypeptide.

In some embodiments, the polypeptide described herein comprises the amino acid sequence GFPLMIKS (SEQ ID NO:6). In some embodiments, the polypeptide described herein comprises the amino acid sequence GFPVMIKS (SEQ ID NO:7). In some embodiments, the polypeptide described herein comprises the amino acid sequence GFPLMIKSASEGGGGK (SEQ ID NO:8). In some embodiments, the polypeptide described herein comprises the amino acid sequence GFPVMIKSASEGGGGK (SEQ ID NO:9). As noted above, in some embodiments of any of these polypeptides, the polypeptides including any one of SEQ ID NOs:6-9: (i) do not comprise a full-length ACC2 amino acid sequence (e.g., from any species); (ii) comprise no more than 2300 consecutive amino acids of an ACC polypeptide from any species); or (iii) comprises no more than 98% of a full-length ACC2 polypeptide.

In some embodiments, the polypeptide comprises an amino acid sequence that is at least 70 (e.g., at least 71, 72, 73, 74, 75, 76, 77, 78, 79, 80, 81, 82, 83, 84, 85, 86, 87, 88, 89, 90, 91, 92, 93, 94, 95, 96, 97, 98, or 99) % identical to at least 8 consecutive amino acids depicted in any one of SEQ ID NOs: 2-5, wherein the polypeptide comprises: (i) the proline at position 450 relative to SEQ ID NO:2 and/or (ii) the amino acid sequence depicted in any one of SEQ ID NOs:6-9. In some embodiments, the polypeptide does not comprise the amino acid sequence of a full-length ACC2 polypeptide (of any isoform from any species). In some embodiments, the polypeptide comprises no more than 98 (e.g., 95, 90, 85, 80, 75, 70, 65, 60, 55, 50, 45, 40, 35, 30, 25, 20 or 15) % of a full-length ACC2 polypeptide. In some embodiments, the polypeptide comprises no more than 2300 (e.g., no more than 2200, 2100, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, or 15) consecutive amino acids of ACC2.

Percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software, such as BLAST software or ClustalW2. Appropriate parameters for measuring alignment, including any algorithms needed to achieve maximal alignment over the full-length of the sequences being compared can be determined by known methods.

In some embodiments, the polypeptide is capable of being hydroxylated at proline 450 and/or proline 343 or 2131) relative to SEQ ID NO:2 by a PHD3 protein—i.e., is a substrate for PHD3. The substrate can be capable of being hydroxlyated by PHD3 in vitro (e.g., using a cell free system) or in cells. In vitro methods for hydroxylating a substrate using PHD3 are exemplified herein. Suitable methods are also described in, e.g., Xie et al. (2012)J Clin Invest 122(8):2827-2836 and Luo et al. (2014) Mol Biol Cell 25(18):2788-2796. For example, a substrate (e.g., a substrate conjugated to a solid support) can be incubated with recombinant PHD3 in a reaction buffer containing 10 μM FeSO₄, 40 μM 2-oxo-glutarate [1-¹⁴C], 1 mM ascorbate, 60 μg catalase, 100 μM dithiothreitol, 2 mg bovine serum albumin, and 50 μM Tris-HCl buffer, adjusted to pH 7.8. The released ¹⁴CO₂ can be detected as a measure of hydroxylation. Alternatively, a substrate, such as any of those described herein, can be incubated with recombinant PHD3 under conditions suitable for hydroxylating a full-length human ACC2 polypeptide. The substrate can be subjected to SDS polyacrylamide gel electrophoresis, followed by western blotting using an antibody that specifically binds to hydroxylated form of ACC2 (described herein).

The disclosure also provides polypeptides comprising all or a portion of ACC2 (e.g., any isoform and from any species, as above), wherein the polypeptide comprises a substitution, modification, or deletion at proline residue 450 relative to SEQ ID NO:2. In some embodiments, the polypeptide comprising all or a portion of ACC2, wherein the proline at position 450 relative to SEQ ID NO:2 is replaced with a different amino acid. In some embodiments, the different amino acid is a non-canonical amino acid. In some embodiments, the different amino acid is a conservative substitution relative to proline. In some embodiments, the different amino acid is a non-conservative substitution relative to proline.

As used herein, the term “conservative substitution” refers to the replacement of an amino acid present in the native sequence in a given polypeptide with a naturally or non-naturally occurring amino acid having similar steric properties. Where the side-chain of the native amino acid to be replaced is either polar or hydrophobic, the conservative substitution should be with a naturally occurring amino acid, a non-naturally occurring amino acid that is also polar or hydrophobic, and, optionally, with the same or similar steric properties as the side-chain of the replaced amino acid. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. One letter amino acid abbreviations are as follows: alanine (A); arginine (R); asparagine (N); aspartic acid (D); cysteine (C); glycine (G); glutamine (Q); glutamic acid (E); histidine (H); isoleucine (I); leucine (L); lysine (K); methionine (M); phenylalanine (F); proline (P); serine (S); threonine (T); tryptophan (W), tyrosine (Y); and valine (V).

The phrase “non-conservative substitution” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted.

In some embodiments, the polypeptide comprises all or part of an ACC2 amino acid sequence in which at least one (e.g., at least two, three, four, five, six, seven, eight, nine, 10, 15, 20, 25, 30, 25, 30, 35, 40, 45, 50, 60, 70, 80, 90, 100, 150, 200, 250, 300, 400, 500, 600, 700, 800, 900, 1000, 1250, 1500, 1750, or 2000) amino acids have been deleted, including the proline at position 450 relative to SEQ ID NO:2. In some embodiments, the polypeptide comprises an ACC2 amino acid sequence comprising at least one amino acid deletion, but no more than 500 (e.g., no more than 450, 400, 350, 300, 250, 200, 150, 100, 90, 80, 70, 60, 50, 40, 30, 20, 15, or 10) deleted consecutive amino acids of the ACC amino acid sequence, wherein the proline at position 450 relative to SEQ ID NO:2 is deleted. The deletion can be at the carboxy-terminus, internal (e.g., one or more amino acid deletions around proline 450 relative to SEQ ID NO:2), or at the amino-terminus of the ACC2 polypeptide.

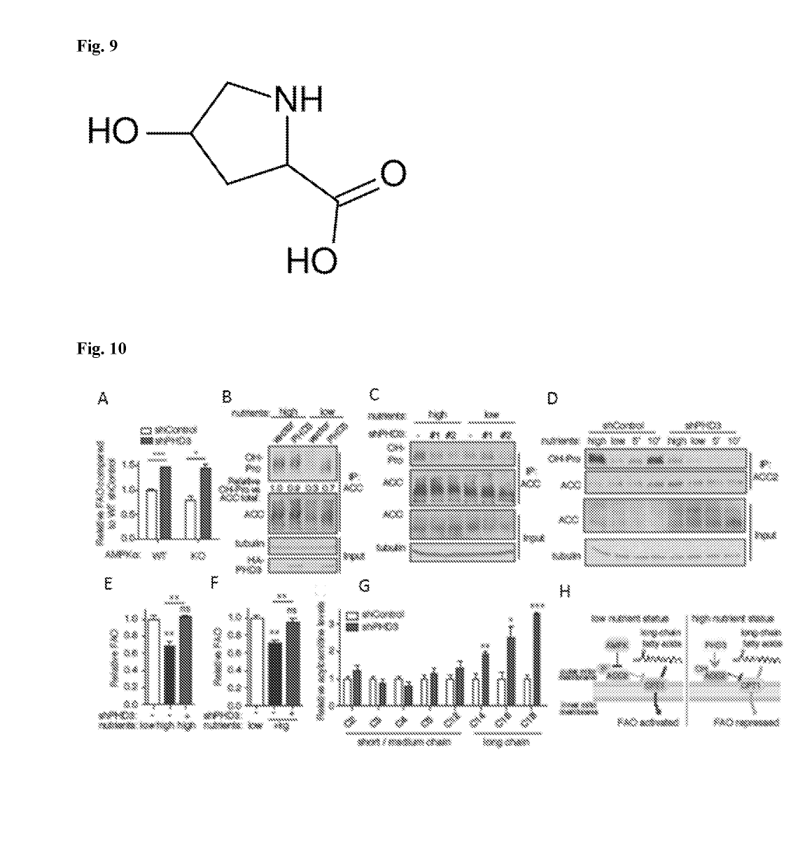

In some embodiments, the polypeptide comprises all or part of an ACC2 amino acid sequence, wherein the proline at position 450 relative to SEQ ID NO:2 is modified. In some embodiments, the proline is hydroxylated (i.e., the gamma carbon atom contains a hydroxyl group) relative to unmodified proline. See FIG. 9.

As noted above, a polypeptide described herein can comprise at least 8 consecutive amino acids of an ACC2 polypeptide (e.g., any isoform from any species), which consecutive amino acids include the modified proline residue at position 450 relative to SEQ ID NO:2, but the polypeptide comprises no more than 2300 (e.g., no more than 2200, 2100, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, or 15) consecutive amino acids of ACC2.

In some embodiments a polypeptide described herein comprises at least 8 consecutive amino acids of an ACC2 polypeptide (of any species), which consecutive amino acids include the modified proline residue at position 450 relative to SEQ ID NO:2, but the polypeptide comprises no more than 98 (e.g., 95, 90, 85, 80, 75, 70, 65, 60, 55, 50, 45, 40, 35, 30, 25, 20 or 15) % of a full-length ACC2 polypeptide.

In some embodiments, the polypeptide described herein comprises the amino acid sequence GFPLMIKS (SEQ ID NO:6) in which the proline is modified. In some embodiments, the polypeptide described herein comprises the amino acid sequence GFPVMIKS (SEQ ID NO:7) in which the proline is modified. In some embodiments, the polypeptide described herein comprises the amino acid sequence GFPLMIKSASEGGGGK (SEQ ID NO:8) in which the proline is modified. In some embodiments, the polypeptide described herein comprises the amino acid sequence GFPVMIKSASEGGGGK (SEQ ID NO:9) in which the proline is modified. In some embodiments of any of these polypeptides, the polypeptides including any one of SEQ ID NOs:6-9: (i) do not comprise a full-length ACC2 amino acid sequence (e.g., from any species); (ii) comprise no more than 2300 consecutive amino acids of an ACC polypeptide from any species); or (iii) comprises no more than 98% of a full-length ACC2 polypeptide.

In some embodiments, the polypeptide comprises or consists of the full-length amino acid sequence of an ACC2 polypeptide (e.g., any isoform from any species), wherein the proline at position 450 relative to SEQ ID NO:2 is modified, e.g., hydroxylated. For example, the polypeptide can comprise or consist of the amino acid sequence depicted in SEQ ID NO:2 in which proline 450 is hydroxylated; the amino acid sequence depicted in SEQ ID NO:3 in which the proline at position 440 is hydroxylated; the amino acid sequence depicted in SEQ ID NO:4 in which the proline at position 446 is hydroxylated; or the amino acid sequence depicted in SEQ ID NO:5 in which the proline at position 371 is hydroxylated.

The disclosure also features polypeptides comprising a portion of ACC2 (e.g., any isoform from any species expressing an ACC2 polypeptide) containing the proline residue at position 343 and/or 2131 (relative to SEQ ID NO:2). Exemplary amino acid sequences for ACC2 polypeptides are set forth herein. The position of prolines 343 and 2131 are set forth below in the context of SEQ ID NO:2:

| | |
|---|---|
| 1 | mvlllclscl ifscltfswl kiwgkmtdsk pitksksean lipsqepfpa sdnsgetpqr |
| 61 | ngeghtlpkt psqaepashk gpkdagrrrn slppshqkpp rnplsssdaa pspelqangt |
| 121 | gtqgleatdt nglsssarpq gqqagspske dkkqanikrq lmtnfilgsf ddyssdedsv |
| 181 | agssrestrk gsraslgals leaylttgea etrvptmrps msglhlvkrg rehkkldlhr |
| 241 | dftvaspaef vtrfggdrvi ekvlianngi aavkcmrsir rwayemfrne rairfvvmvt |
| 301 | pedlkanaey ikmadhyvpv pggpnnnnya nvelivdiak ripvqavwag wghasenpkl |
| 361 | pellckngva flgppseamw algdkiastv vaqtlqvptl pwsgsgltve wteddlqqgk |
| 421 | risvpedvyd kgcvkdvdeg leaaerigfp lmikaseggg gkgirkaesa edfpilfrqv |
| 481 | qseipgspif lmklaqharh levqiladqy gnayslfgrd csiqrrhqki veeapatiap |
| 541 | laifefmeqc airlaktvgy vsagtveyly sqdgsfhfle lnprlqvehp ctemiadvnl |
| 601 | paaqlqiamg vplhrlkdir llygespwgv tpisfetpsn pplarghvia aritsenpde |
| 661 | gfkpssgtvq elnfrssknv wgyfsvaatg glhefadsqf ghcfswgenr eeaisnmvva |
| 721 | lkelsirgdf rttveylinl letesfqnnd idtgwldyli aekvqaekpd imlgvvcgal |
| 781 | nvadamfrtc mtdflhsler gqvlpadsll nlvdveliyg gvkyilkvar qsltmfvlim |
| 841 | ngchieidah rindggllls yngnsyttym keevdsyrit ignktcvfek endptvlrsp |
| 901 | sagkltqytv edgghveags syaemevmkm imtlnvqerg rvkyikrpga vleagcvvar |
| 961 | lelddpskvh paepftgelp aqqtlpilge klhqvfhsvl enltnvmsgf clpepvfsik |
| 1021 | lkewvqklmm tlrhpslpll elqeimtsva gripapveks vrrvmaqyas nitsvlcqfp |
| 1081 | sqqiatildc haatlqrkad revffintqs ivqlvqryrs girgymktvv ldllrrylrv |
| 1141 | ehhfqqahyd kcvinlreqf kpdmsqvldc ifshaqvakk nqlvimlide lcgpdpslsd |
| 1201 | elisilnelt qlsksehckv alrarqilia shlpsyelrh nqvesiflsa idmyghqfcp |
| 1261 | enlkklilse ttifdvlptf fyhankvvcm aslevyvrrg yiayelnslq hrqlpdgtcv |
| 1321 | vefqfmlpss hpnrmtvpis itnpdllrhs telfmdsgfs plcqrmgamv afrrfedftr |
| 1381 | nfdeviscfa nvpkdtplfs eartslysed dckslreepi hilnvsiqca dhledealvp |
| 1441 | ilrtfvqskk nilvdyglrr itfliaqeke fpkfftfrar defaedriyr hlepalafql |
| 1501 | elnrmrnfdl tavpcanhkm hlylgaakvk egvevtdhrf firaiirhsd litkeasfey |
| 1561 | lqnegerlll eamdelevaf nntsvrtdcn hiflnfvptv imdpfkiees vrymvmrygs |
| 1621 | rlwklrvlqa evkinirqtt tgsavpirlf itnesgyyld islykevtds rsgnimfhsf |
| 1681 | gnkqgpqhgm lintpyvtkd llqakrfqaq tlgttyiydf pemfrqalfk lwgspdkypk |
| 1741 | diltytelvl dsqgqlvemn rlpggnevgm vafkmrfktq eypegrdviv ignditfrig |
| 1801 | sfgpgedlly lrasemarae gipkiyvaan sgarigmaee ikhmfhvawv dpedphkgfk |
| 1861 | ylyltpqdyt risslnsvhc khieeggesr ymitdiigkd dglgvenlrg sgmiagessl |
| 1921 | ayeeivtisl vtcraigiga ylvrlgqrvi qvenshiilt gasalnkvlg revytsnnql |
| 1981 | ggvqimhyng vshitvpddf egvytilewl sympkdnhsp vpiitptdpi dreieflpsr |
| 2041 | apydprwmla grphptlkgt wqsgffdhgs fkeimapwaq tvvtgrarlg gipvgviave |
| 2101 | trtvevavpa dpanldseak iiqqagqvwf pdsayktaqa vkdfnreklp lmifanwrgf |
| 2161 | sggmkdmydq vlkfgayivd glrqykqpil iyippyaelr ggswvvidat inplciemya |
| 2221 | dkesrggvle pegtveikfr kkdliksmrr idpaykklme qlgepdlsdk drkdlegrlk |
| 2281 | aredlllpiy hqvavqfadf hdtpgrmlek gvisdilewk tartflywrl rrllledqvk |
| 2341 | qeilqasgel shvhiqsmlr rwfvetegav kaylwdnnqv vvqwleqhwq agdgprstir |
| 2401 | enitylkhds vlktirglve enpevavdcv iylsqhispa eraqvvhlls tmdspast |

In some embodiments, a polypeptide described herein comprises at least 8 (e.g., at least 9, 10, 11, 12, 13, 14, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1250, 1500, 1775, or 2000) consecutive amino acids of an ACC2 polypeptide (of any species), which consecutive amino acids include the proline residue at position 343, 450, and/or 2131 relative to SEQ ID NO:2, but the polypeptide does not comprise the entire amino acid sequence of ACC2.

In some embodiments a polypeptide described herein comprises at least 8 consecutive amino acids of an ACC2 polypeptide (of any species), which consecutive amino acids include the proline residue at position 343, 450, and/or 2131 relative to SEQ ID NO:2, but the polypeptide comprises no more than 2300 (e.g., no more than 2200, 2100, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, or 15) consecutive amino acids of ACC2.

In some embodiments a polypeptide described herein comprises at least 8 consecutive amino acids of an ACC2 polypeptide (of any species), which consecutive amino acids include the proline residue at position 343, 450, and/or 2131 relative to SEQ ID NO:2, but the polypeptide comprises no more than 98 (e.g., 95, 90, 85, 80, 75, 70, 65, 60, 55, 50, 45, 40, 35, 30, 25, 20 or 15) % of a full-length ACC2 polypeptide.

As above, in some embodiments, the polypeptide comprising the proline residue at position 343, 450, and/or 2131 relative to SEQ ID NO:2 can be capable of being hydroxylated at by a PHD3 protein—i.e., is a substrate for PHD3. The substrate can be hydroxlyated by PHD3 in vitro (e.g., using a cell free system) or in cells. In vitro and in vivo methods for hydroxylating a substrate using PHD3 are described herein.

The disclosure also provides polypeptides comprising all or a portion of ACC2 (e.g., any isoform and from any species, as above), wherein the polypeptide comprises a substitution (replacement), modification, or deletion of the proline residue at one or more prolines at position 343, 450, and 2131 relative to SEQ ID NO:2. In some embodiments, the polypeptide comprising all or a portion of ACC2, wherein the proline one or more of positions 343, 450, and 2131 relative to SEQ ID NO:2 are replaced with a different amino acid. In some embodiments, the different amino acid is a non-canonical amino acid. In some embodiments, the different amino acid is a conservative substitution relative to proline. In some embodiments, the different amino acid is a non-conservative substitution relative to proline.

In some embodiments, the polypeptide comprises all or part of an ACC2 amino acid sequence in which at least one (e.g., at least two, three, four, five, six, seven, eight, nine, 10, 15, 20, 25, 30, 25, 30, 35, 40, 45, 50, 60, 70, 80, 90, 100, 150, 200, 250, 300, 400, 500, 600, 700, 800, 900, 1000, 1250, 1500, 1750, or 2000) amino acids have been deleted, including one or more of the prolines at positions 343, 450, and/or 2131 relative to SEQ ID NO:2. In some embodiments, the polypeptide comprises an ACC2 amino acid sequence comprising at least one amino acid deletion, but no more than 500 (e.g., no more than 450, 400, 350, 300, 250, 200, 150, 100, 90, 80, 70, 60, 50, 40, 30, 20, 15, or 10) deleted consecutive amino acids of the ACC amino acid sequence, wherein the proline at position 343, 450, and/or 2131 relative to SEQ ID NO:2 are deleted. The deletion can be at the carboxy-terminus, internal (e.g., one or more amino acid deletions around one or more prolines at positions 343, 450, and 2131 relative to SEQ ID NO:2), or at the amino-terminus of the ACC2 polypeptide.

In some embodiments, the polypeptide comprises all or part of an ACC2 amino acid sequence, wherein the proline at one or more of positions 343, 450, and 2131 relative to SEQ ID NO:2 is modified. In some embodiments, the proline is hydroxylated (i.e., the gamma carbon atom contains a hydroxyl group) relative to unmodified proline. See FIG. 9.

As noted above, a polypeptide described herein can comprise at least 8 consecutive amino acids of an ACC2 polypeptide (e.g., any isoform from any species), which consecutive amino acids include the modified proline residue at one or more positions 343, 450, and 2131 relative to SEQ ID NO:2, but the polypeptide comprises no more than 2300 (e.g., no more than 2200, 2100, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, or 15) consecutive amino acids of ACC2.

In some embodiments a polypeptide described herein comprises at least 8 consecutive amino acids of an ACC2 polypeptide (of any species), which consecutive amino acids include the modified proline residue at one or more positions 343, 450, and/or 2131 relative to SEQ ID NO:2, but the polypeptide comprises no more than 98 (e.g., 95, 90, 85, 80, 75, 70, 65, 60, 55, 50, 45, 40, 35, 30, 25, 20 or 15) % of a full-length ACC2 polypeptide.

In some embodiments, the polypeptide comprises or consists of the full-length amino acid sequence of an ACC2 polypeptide (e.g., any isoform from any species), wherein the proline at position 343, 450, and/or 2131 relative to SEQ ID NO:2 is modified, e.g., hydroxylated. For example, the polypeptide can comprise or consist of the amino acid sequence depicted in SEQ ID NO:2 in which proline 343, 450, and/or 2131 is hydroxylated.

In some embodiments, a polypeptide described herein can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK (SEQ ID NO:10)), polyhistidine (6-His; HHHHHH (SEQ ID NO:11), hemagglutinin (HA; YPYDVPDYA (SEQ ID NO:12)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins can be cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of a protein agent. Alternatively, the radioactive label can be included as part of a larger molecule (e.g., ¹²⁵I in meta-[¹²⁵I]iodophenyl-N-hydroxysuccinimide ([¹²⁵I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein, or fragment thereof, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the agents can be modified, e.g., with a moiety that improves the stabilization and/or retention of the antibodies in circulation, e.g., in blood, serum, or other tissues. For example, a polypeptide described herein can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476 or HESylated (Fresenius Kabi, Germany; see, e.g., Pavisić et al. (2010) Int J Pharm 387(1-2): 110-119). The stabilization moiety can improve the stability, or retention of, the polypeptide by at least 1.5 (e.g., at least 2, 5, 10, 15, 20, 25, 30, 40, or 50 or more) fold.

In some embodiments, the polypeptides can be fusion proteins having at least a portion of an ACC2 polypeptide and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. As another example, a fusion domain may be selected so as to facilitate detection of the polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some embodiments, the fusion proteins comprise a linker moiety of one or more amino acids separating the ACC2 polypeptide (variant or functional fragment) portion and the heterologous portion (e.g., the Fc region or albumin molecule). In some embodiments, the linker region comprises a polyglycine sequence or poly (GS) sequence. In some cases, the fusion domains have a protease cleavage site, such as for Factor Xa, Thrombin, or Tobacco Etch Virus (TEV) protease, which allows the relevant protease to partially digest the fusion proteins and thereby liberate the recombinant proteins therefrom. The liberated proteins can then be isolated from the fusion domain by subsequent chromatographic separation. In some embodiments, a polypeptide described herein (e.g., comprising all of part of an ACC2 polypeptide, optionally with a modification, substitution, or deletion at proline 450 relative to SEQ ID NO:2) can be fused with a domain that stabilizes the ACC2 polypeptide in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains.

Fc regions may be derived from antibodies belonging to each of the immunoglobulin classes referred to as IgA, IgD, IgE, IgG (e.g., subclasses IgG1, IgG2, IgG3, and IgG4), and IgM. The choice of appropriate Fc regions is discussed in detail in U.S. Pat. Nos. 5,541,087, and 5,726,044, the disclosures of which are incorporated herein by reference in their entirety. It may be useful, in some circumstances, to modify the immunoglobulin heavy chain constant region, for example, by mutation, deletion or other changes mediated by genetic engineering or other approaches, so that certain activities, such as complement fixation or stimulation of antibody-dependent cell-mediated cytotoxicity (ADCC) are reduced or eliminated, while preferably preserving the Fc regions' ability to bind an Fc receptor (e.g., FcRn).

In some embodiments, the Fc region (including those of an antibody or antigen-binding fragment described herein) can be an altered Fc constant region having reduced (or no) effector function relative to its corresponding unaltered constant region. Effector functions involving the Fc constant region may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent. For example, an altered Fc constant region that displays modulated ADCC and/or CDC activity may exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the Fc constant region. An altered Fc region described herein may exhibit reduced or no measurable ADCC and/or CDC activity.

In certain embodiments, the altered constant region has at least one amino acid substitution, insertion, and/or deletion, compared to a native sequence constant region or to the unaltered constant region, e.g. from about one to about one hundred amino acid substitutions, insertions, and/or deletions in a native sequence constant region or in the constant region of the parent polypeptide. In some embodiments, the altered constant region herein will possess at least about 70% homology (similarity) or identity with the unaltered constant region and in some instances at least about 75% and in other instances at least about 80% homology or identity therewith, and in other embodiments at least about 85%, 90% or 95% homology or identity therewith. The altered constant region may also contain one or more amino acid deletions or insertions. Additionally, the altered constant region may contain one or more amino acid substitutions, deletions, or insertions that results in altered post-translational modifications, including, for example, an altered glycosylation pattern (e.g., the addition of one or more sugar components, the loss of one or more sugar components, or a change in composition of one or more sugar components relative to the unaltered constant region).

### Polypeptide Expression

A recombinant polypeptide (e.g., fragments or fusion proteins) can be produced using a variety of techniques known in the art of molecular biology and protein chemistry. For example, a nucleic acid encoding a fusion protein can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of recombinant polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of recombinant proteins include yeast, bacteria, insect, plant, and mammalian cells (e.g., rodent cell lines, such as Chinese Hamster Ovary (CHO) cells). Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, a recombinant protein can be expressed in, and purified from, transgenic animals (e.g., transgenic mammals). For example, a recombinant protein can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999) J Immunol Methods 231(1-2): 147-157.

A polypeptide can be produced from the cells by culturing a host cell transformed with the expression vector containing nucleic acid encoding the polypeptide, under conditions, and for an amount of time, sufficient to allow expression of the proteins. Such conditions for protein expression will vary with the choice of the expression vector and the host cell, and will be easily ascertained by one skilled in the art through routine experimentation. For example, proteins expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. A fusion protein described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the recombinant proteins can be isolated. The term “purified” or “isolated” as applied to any of the proteins described herein refers to a polypeptide that has been separated or purified from components (e.g., proteins or other naturally-occurring biological or organic molecules) which naturally accompany it, e.g., other proteins, lipids, and nucleic acid in a prokaryotic or eukaryotic cell expressing the proteins. Typically, a polypeptide is purified when it constitutes at least 60 (e.g., at least 65, 70, 75, 80, 85, 90, 92, 95, 97, or 99) %, by weight, of the total protein in a sample.

The recombinant proteins can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed proteins will be necessary.

Methods for determining the yield or purity of a purified protein are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some embodiments, endotoxin can be removed from the protein preparations. Methods for removing endotoxin from a protein sample are known in the art and exemplified in the working examples. For example, endotoxin can be removed from a protein sample using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Mirus), or Acrodisc™-Mustang® E membrane (Pall Corporation).

Methods for detecting and/or measuring the amount of endotoxin present in a sample (both before and after purification) are known in the art and commercial kits are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and CSE kits available from the Associates of Cape Cod Incorporated.

### Antibodies

Also featured herein are antibodies that bind to ACC2 polypeptides that are modified at proline 343, 450, and/or 2131 relative to SEQ ID NO:2, e.g., an ACC2 polypeptide hydroxylated at proline 450 relative to SEQ ID NO:2. As used herein, the term “antibody” refers to whole antibodies including antibodies of different isotypes, such as IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

Antibodies also include antigen-binding fragments (referred to herein as “antibody fragment” and “antigen-binding fragment,” or similar terms) which are fragments of an antibody that retain the ability to bind to an target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12) 1121-1123; Rondon and Marasco (1997) Annual Review of Microbiology 51:257-283, the disclosures of each of which are incorporated herein by reference in their entirety. Bispecific antibodies (including DVD-Ig antibodies; see below) are also embraced by the term “antibody.” Bispecific antibodies are monoclonal, preferably human or humanized, antibodies that have binding specificities for at least two different antigens.

As used in herein, the term “antibody” also includes, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed.

Suitable methods for producing an antibody, or antigen-binding fragments thereof, in accordance with the disclosure are known in the art and described herein. For example, monoclonal antibodies may be generated using cells that express a target antigen of interest, a target antigen (e.g., all or part of an ACC2 polypeptide containing hydroxylated proline at position 450 relative to SEQ ID NO:2) of interest itself, or an antigenic fragment of the target antigen, as an immunogen, thus raising an immune response in animals from which antibody-producing cells and in turn monoclonal antibodies may be isolated. The sequence of such antibodies may be determined and the antibodies or variants thereof produced by recombinant techniques. Recombinant techniques may be used to produce chimeric, CDR-grafted, humanized and fully human antibodies based on the sequence of the monoclonal antibodies as well as polypeptides capable of binding to the target antigen. The amino acid sequences for exemplary ACC2 polypeptides are known in the art and described herein.

Moreover, antibodies derived from recombinant libraries (“phage antibodies”) may be selected using target antigen-expressing cells, or polypeptides derived therefrom, as bait to isolate the antibodies or polypeptides on the basis of target specificity. The production and isolation of non-human and chimeric antibodies are well within the purview of the skilled artisan.

Recombinant DNA technology can be used to modify one or more characteristics of the antibodies produced in non-human cells. Thus, chimeric antibodies can be constructed in order to decrease the immunogenicity thereof in diagnostic or therapeutic applications. Moreover, immunogenicity can be minimized by humanizing the antibodies by CDR grafting and, optionally, framework modification. See, U.S. Pat. Nos. 5,225,539 and 7,393,648, the contents of each of which are incorporated herein by reference.

Antibodies can be obtained from animal serum or, in the case of monoclonal antibodies or fragments thereof, produced in cell culture. Recombinant DNA technology can be used to produce the antibodies according to established procedure, including procedures in bacterial or preferably mammalian cell culture. The selected cell culture system preferably secretes the antibody product.

In another embodiment, a process for the production of an antibody disclosed herein includes culturing a host, e.g., E. coli or a mammalian cell, which has been transformed with a hybrid vector. The vector includes one or more expression cassettes containing a promoter operably linked to a first DNA sequence encoding a signal peptide linked in the proper reading frame to a second DNA sequence encoding the antibody protein. The antibody protein is then collected and isolated. Optionally, the expression cassette may include a promoter operably linked to polycistronic (e.g., bicistronic) DNA sequences encoding antibody proteins each individually operably linked to a signal peptide in the proper reading frame.

Multiplication of hybridoma cells or mammalian host cells in vitro is carried out in suitable culture media, which include the customary standard culture media (such as, for example Dulbecco's Modified Eagle Medium (DMEM) or RPMI 1640 medium), optionally replenished by a mammalian serum (e.g. fetal calf serum), or trace elements and growth sustaining supplements (e.g. feeder cells such as normal mouse peritoneal exudate cells, spleen cells, bone marrow macrophages, 2-aminoethanol, insulin, transferrin, low density lipoprotein, oleic acid, or the like). Multiplication of host cells which are bacterial cells or yeast cells is likewise carried out in suitable culture media known in the art. For example, for bacteria suitable culture media include medium LE, NZCYM, NZYM, NZM, Terrific Broth, SOB, SOC, 2×YT, or M9 Minimal Medium. For yeast, suitable culture media include medium YPD, YEPD, Minimal Medium, or Complete Minimal Dropout Medium.

In vitro production provides relatively pure antibody preparations and allows scale-up production to give large amounts of the desired antibodies. Techniques for bacterial cell, yeast, plant, or mammalian cell cultivation are known in the art and include homogeneous suspension culture (e.g. in an airlift reactor or in a continuous stirrer reactor), and immobilized or entrapped cell culture (e.g. in hollow fibers, microcapsules, on agarose microbeads or ceramic cartridges).

Large quantities of the desired antibodies can also be obtained by multiplying mammalian cells in vivo. For this purpose, hybridoma cells producing the desired antibodies are injected into histocompatible mammals to cause growth of antibody-producing tumors. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane. After one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, the disclosures of which are all incorporated herein by reference. Techniques for the preparation of recombinant antibody molecules are described in the above references and also in, e.g.: WO97/08320; U.S. Pat. No. 5,427,908; U.S. Pat. No. 5,508,717; Smith (1985) Science 225:1315-1317; Parmley and Smith (1988) Gene 73:305-318; De La Cruz et al. (1988) J Biol Chem 263:4318-4322; U.S. Pat. No. 5,403,484; U.S. Pat. No. 5,223,409; WO88/06630; WO92/15679; U.S. Pat. No. 5,780,279; U.S. Pat. No. 5,571,698; U.S. Pat. No. 6,040,136; Davis et al. (1999) Cancer Metastasis Rev 18(4):421-5; Taylor et al. (1992) Nucleic Acids Res 20: 6287-6295; and Tomizuka et al. (2000) Proc Natl Acad Sci USA 97(2): 722-727, the contents of each of which are incorporated herein by reference in their entirety.

The cell culture supernatants are screened for the desired antibodies, e.g., by immunofluorescent staining of target antigen-expressing cells, by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g., by precipitation with ammonium sulfate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with one or more surface polypeptides derived from a target antigen-expressing cell line, or with Protein-A or -G.

Another embodiment provides a process for the preparation of a bacterial cell line secreting antibodies directed against a target antigen in a suitable mammal. For example, a rabbit is immunized with pooled samples from target antigen-expressing tissue or cells or the target antigen itself (or fragments thereof). A phage display library produced from the immunized rabbit is constructed and panned for the desired antibodies in accordance with methods well known in the art (such as, e.g., the methods disclosed in the various references incorporated herein by reference).

Hybridoma cells secreting the monoclonal antibodies are also disclosed. The preferred hybridoma cells are genetically stable, secrete monoclonal antibodies described herein of the desired specificity, and can be expanded from deep-frozen cultures by thawing and propagation in vitro or as ascites in vivo.

In another embodiment, a process is provided for the preparation of a hybridoma cell line secreting monoclonal antibodies against a target antigen of interest. In that process, a suitable mammal, for example a Balb/c mouse, is immunized with, e.g., a target antigen of interest (or an antigenic fragment thereof) as described. Antibody-producing cells of the immunized mammal are grown briefly in culture or fused with cells of a suitable myeloma cell line. The hybrid cells obtained in the fusion are cloned, and cell clones secreting the desired antibodies are selected. The obtained hybrid cells are then screened for secretion of the desired antibodies and positive hybridoma cells are cloned.

Methods for preparing a hybridoma cell line include immunizing Balb/c mice by injecting subcutaneously and/or intraperitoneally an immunogenic composition several times, e.g., four to six times, over several months, e.g., between two and four months. Spleen cells from the immunized mice are taken two to four days after the last injection and fused with cells of the myeloma cell line PAI in the presence of a fusion promoter, preferably polyethylene glycol. Preferably, the myeloma cells are fused with a three- to twenty-fold excess of spleen cells from the immunized mice in a solution containing about 30% to about 50% polyethylene glycol of a molecular weight around 4000. After the fusion, the cells are expanded in suitable culture media as described supra, supplemented with a selection medium, for example HAT medium, at regular intervals in order to prevent normal myeloma cells from overgrowing the desired hybridoma cells.

The antibodies and fragments thereof can be “chimeric.” Chimeric antibodies and antigen-binding fragments thereof comprise portions from two or more different species (e.g., mouse and human). Chimeric antibodies can be produced with mouse variable regions of desired specificity spliced onto human constant domain gene segments (for example, U.S. Pat. No. 4,816,567). In this manner, non-human antibodies can be modified to make them more suitable for human clinical application (e.g., methods for treating or preventing an immune associated disorder in a human subject).

The monoclonal antibodies of the present disclosure include “humanized” forms of the non-human (e.g., mouse) antibodies. Humanized or CDR-grafted mAbs are particularly useful as therapeutic agents for humans because they are not cleared from the circulation as rapidly as mouse antibodies and do not typically provoke an adverse immune reaction. Methods of preparing humanized antibodies are generally well known in the art. For example, humanization can be essentially performed following the method of Winter and co-workers (see, e.g., Jones et al. (1986) Nature 321:522-525; Riechmann et al. (1988) Nature 332:323-327; and Verhoeyen et al. (1988) Science 239:1534-1536), by substituting rodent CDRs or CDR sequences for the corresponding sequences of a human antibody. Also see, e.g., Staelens et al. (2006) Mol Immunol 43:1243-1257. In some embodiments, humanized forms of non-human (e.g., mouse) antibodies are human antibodies (recipient antibody) in which hypervariable (CDR) region residues of the recipient antibody are replaced by hypervariable region residues from a non-human species (donor antibody) such as a mouse, rat, rabbit, or non-human primate having the desired specificity, affinity, and binding capacity. In some instances, framework region residues of the human immunoglobulin are also replaced by corresponding non-human residues (so called “back mutations”). In addition, phage display libraries can be used to vary amino acids at chosen positions within the antibody sequence. The properties of a humanized antibody are also affected by the choice of the human framework. Furthermore, humanized and chimerized antibodies can be modified to comprise residues that are not found in the recipient antibody or in the donor antibody in order to further improve antibody properties, such as, for example, affinity or effector function.

Fully human antibodies are also provided in the disclosure. The term “human antibody” includes antibodies having variable and constant regions (if present) derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences (i.e., humanized antibodies). Fully human or human antibodies may be derived from transgenic mice carrying human antibody genes (carrying the variable (V), diversity (D), joining (J), and constant (C) exons) or from human cells. For example, it is now possible to produce transgenic animals (e.g., mice) that are capable, upon immunization, of producing a full repertoire of human antibodies in the absence of endogenous immunoglobulin production. (See, e.g., Jakobovits et al. (1993) Proc Natl Acad Sci USA 90:2551; Jakobovits et al. (1993) Nature 362:255-258; Bruggemann et al. (1993) Year in Immunol 7:33; and Duchosal et al. (1992) Nature 355:258.) Transgenic mice strains can be engineered to contain gene sequences from unrearranged human immunoglobulin genes. The human sequences may code for both the heavy and light chains of human antibodies and would function correctly in the mice, undergoing rearrangement to provide a wide antibody repertoire similar to that in humans. The transgenic mice can be immunized with the target antigen to create a diverse array of specific antibodies and their encoding RNA. Nucleic acids encoding the antibody chain components of such antibodies may then be cloned from the animal into a display vector. Typically, separate populations of nucleic acids encoding heavy and light chain sequences are cloned, and the separate populations then recombined on insertion into the vector, such that any given copy of the vector receives a random combination of a heavy and a light chain. The vector is designed to express antibody chains so that they can be assembled and displayed on the outer surface of a display package containing the vector. For example, antibody chains can be expressed as fusion proteins with a phage coat protein from the outer surface of the phage. Thereafter, display packages can be screened for display of antibodies binding to a target.

In addition, human antibodies can be derived from phage-display libraries (Hoogenboom et al. (1991) J Mol Biol 227:381; Marks et al. (1991) J Mol Biol 222:581-597; and Vaughan et al. (1996) Nature Biotech 14:309 (1996)). Synthetic phage libraries can be created which use randomized combinations of synthetic human antibody V-regions. By selection on antigen fully human antibodies can be made in which the V-regions are very human-like in nature. See, e.g., U.S. Pat. Nos. 6,794,132; 6,680,209; and 4,634,666, and Ostberg et al. (1983) Hybridoma 2:361-367, the contents of each of which are incorporated herein by reference in their entirety.

For the generation of human antibodies, also see Mendez et al. (1998) Nature Genetics 15:146-156 and Green and Jakobovits (1998) J Exp Med 188:483-495, the disclosures of which are hereby incorporated by reference in their entirety. Human antibodies are further discussed and delineated in U.S. Pat. Nos. 5,939,598; 6,673,986; 6,114,598; 6,075,181; 6,162,963; 6,150,584; 6,713,610; and 6,657,103 as well as U.S. Patent Application Publication Nos. 20030229905 A1, 20040010810 A1, 20040093622 A1, 20060040363 A1, 20050054055 A1, 20050076395 A1, and 20050287630 A1. See also International Patent Application Publication Nos. WO 94/02602, WO 96/34096, and WO 98/24893, and European Patent No. EP 0 463 151 B1. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in, e.g., U.S. Pat. Nos. 5,545,807; 5,545,806; 5,625,825; 5,625,126; 5,633,425; 5,661,016; 5,770,429; 5,789,650; 5,814,318; 5,591,669; 5,612,205; 5,721,367; 5,789,215; 5,643,763; 5,569,825; 5,877,397; 6,300,129; 5,874,299; 6,255,458; and 7,041,871, the disclosures of which are hereby incorporated by reference. See also European Patent No. 0 546 073 B1, International Patent Application Publication Nos. WO 92/03918, WO 92/22645, WO 92/22647, WO 92/22670, WO 93/12227, WO 94/00569, WO 94/25585, WO 96/14436, WO 97/13852, and WO 98/24884, the disclosures of each of which are hereby incorporated by reference in their entirety. See further Taylor et al. (1992) Nucleic Acids Res 20: 6287; Chen et al. (1993) Int Immunol 5:647; Tuaillon et al. (1993) Proc Natl Acad Sci USA 90: 3720-4; Choi et al. (1993) Nature Genetics 4: 117; Lonberg et al. (1994) Nature 368: 856-859; Taylor et al. (1994) Int Immunol 6: 579-591; Tuaillon et al. (1995) J Immunol 154: 6453-65; Fishwild et al. (1996) Nature Biotechnol 14: 845; and Tuaillon et al. (2000) Eur J Immunol 10: 2998-3005, the disclosures of each of which are hereby incorporated by reference in their entirety.

In certain embodiments, de-immunized antibodies or antigen-binding fragments thereof are provided. De-immunized antibodies or antigen-binding fragments thereof are antibodies that have been modified so as to render the antibody or antigen-binding fragment thereof non-immunogenic, or less immunogenic, to a given species (e.g., to a human). De-immunization can be achieved by modifying the antibody or antigen-binding fragment thereof utilizing any of a variety of techniques known to those skilled in the art (see, e.g., PCT Publication Nos. WO 04/108158 and WO 00/34317). For example, an antibody or antigen-binding fragment thereof may be de-immunized by identifying potential T cell epitopes and/or B cell epitopes within the amino acid sequence of the antibody or antigen-binding fragment thereof and removing one or more of the potential T cell epitopes and/or B cell epitopes from the antibody or antigen-binding fragment thereof, for example, using recombinant techniques. The modified antibody or antigen-binding fragment thereof may then optionally be produced and tested to identify antibodies or antigen-binding fragments thereof that have retained one or more desired biological activities, such as, for example, binding affinity, but have reduced immunogenicity. Methods for identifying potential T cell epitopes and/or B cell epitopes may be carried out using techniques known in the art, such as, for example, computational methods (see e.g., PCT Publication No. WO 02/069232), in vitro or in silico techniques, and biological assays or physical methods (such as, for example, determination of the binding of peptides to MHC molecules, determination of the binding of peptide:MHC complexes to the T cell receptors from the species to receive the antibody or antigen-binding fragment thereof, testing of the protein or peptide parts thereof using transgenic animals with the MHC molecules of the species to receive the antibody or antigen-binding fragment thereof, or testing with transgenic animals reconstituted with immune system cells from the species to receive the antibody or antigen-binding fragment thereof, etc.). In various embodiments, the de-immunized antibodies described herein include de-immunized antigen-binding fragments, Fab, Fv, scFv, Fab′ and F(ab′)₂, monoclonal antibodies, murine antibodies, engineered antibodies (such as, for example, chimeric, single chain, CDR-grafted, humanized, and artificially selected antibodies), synthetic antibodies and semi-synthetic antibodies.

In some embodiments, a recombinant DNA comprising an insert coding for a heavy chain variable domain and/or for a light chain variable domain of an antibody is produced. The term DNA includes coding single stranded DNAs, double stranded DNAs consisting of said coding DNAs and of complementary DNAs thereto, or these complementary (single stranded) DNAs themselves.

Furthermore, a DNA encoding a heavy chain variable domain and/or a light chain variable domain of antibodies can be enzymatically or chemically synthesized to contain the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted, inserted, or exchanged with one or more other amino acids. Preferably said modification(s) are outside the CDRs of the heavy chain variable domain and/or the CDRs of the light chain variable domain of the antibody in humanization and expression optimization applications. The term mutant DNA also embraces silent mutants wherein one or more nucleotides are replaced by other nucleotides with the new codons coding for the same amino acid(s). The term mutant sequence also includes a degenerate sequence. Degenerate sequences are degenerate within the meaning of the genetic code in that an unlimited number of nucleotides are replaced by other nucleotides without resulting in a change of the amino acid sequence originally encoded. Such degenerate sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

Recombinant DNAs including an insert coding for a heavy chain murine variable domain of an antibody-expressing cell line fused to a human constant domain IgG, for example γ1, γ2, γ3 or γ4, in particular embodiments γ1 or γ4, may be used. Recombinant DNAs including an insert coding for a light chain murine variable domain of an antibody fused to a human constant domain κ or λ, preferably κ, are also provided.

Another embodiment pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA sequence encoding a peptide facilitating the purification of the antibody and/or a cleavage site and/or a peptide spacer and/or an agent.

Accordingly, the monoclonal antibodies or antigen-binding fragments of the disclosure can be naked antibodies or antigen-binding fragments that are not conjugated to other agents, for example, a therapeutic agent or detectable label. Alternatively, the monoclonal antibody or antigen-binding fragment can be conjugated to an agent such as, for example, a cytotoxic agent, a small molecule, a hormone, an enzyme, a growth factor, a cytokine, a ribozyme, a peptidomimetic, a chemical, a prodrug, a nucleic acid molecule including coding sequences (such as antisense, RNAi, gene-targeting constructs, etc.), or a detectable label (e.g., an NMR or X-ray contrasting agent, fluorescent molecule, etc.). In certain embodiments, an antibody or antigen-binding fragment (e.g., Fab, Fv, single-chain (scFv), Fab′, and F(ab′)₂) is linked to a molecule that increases the half-life of the antibody or antigen-binding fragment (see above).

Several possible vector systems are available for the expression of cloned heavy chain and light chain genes in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA, 78:2072-2076) or Tn5 neo (Southern and Berg (1982) J Mol Appl Genet 1:327-341). The selectable marker gene can be either linked to the DNA gene sequences to be expressed, or introduced into the same cell by co-transfection (Wigler et al. (1979) Cell 16:777-785). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147-7151), polyoma virus (Deans et al. (1984) Proc Natl Acad Sci USA 81:1292-1296), or SV40 virus (Lusky and Botchan (1981) Nature 293:79-81).

Since an immunoglobulin cDNA is comprised only of sequences representing the mature mRNA encoding an antibody protein, additional gene expression elements regulating transcription of the gene and processing of the RNA are required for the synthesis of immunoglobulin mRNA. These elements may include splice signals, transcription promoters, including inducible promoters, enhancers, and termination signals. cDNA expression vectors incorporating such elements include those described by Okayama and Berg (1983) Mol Cell Biol 3:280-289; Cepko et al. (1984) Cell 37:1053-1062; and Kaufman (1985) Proc Natl Acad Sci USA 82:689-693.

Bispecific antibodies are monoclonal, preferably human or humanized, antibodies that have binding specificities for at least two different epitopes. Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello (1983) Nature 305:537-539). Antibody variable domains with the desired binding specificities (antibody-antigen combining sites) can be fused to immunoglobulin constant domain sequences. The fusion preferably is with an immunoglobulin heavy-chain constant domain, including at least part of the hinge, C_{H}2, and C_{H}3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, e.g., Suresh et al. (1986)Methods Enzymol 121:210-228; PCT Publication No. WO 96/27011; Brennan et al. (1985) Science 229:81-83; Shalaby et al. J Exp Med(1992) 175:217-225; Kostelny et al. (1992) J Immunol 148(5):1547-1553; Hollinger et al. (1993) Proc Natl Acad Sci USA 90:6444-6448; Gruber et al. (1994) J Immunol 152:5368-5474; and Tutt et al. (1991) J Immunol 147:60-69. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368-5374. Alternatively, the antibodies can be “linear antibodies” as described in, e.g., Zapata et al. (1995) Protein Eng 8(10): 1057-1062. Briefly, these antibodies comprise a pair of tandem Fd segments (V_{H}-C_{H}1-V_{H}-C_{H}1) which form a pair of antigen binding regions. Linear antibodies can be bispecific or monospecific.

The disclosure also embraces variant forms of bispecific antibodies such as the tetravalent dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. The light chain is paired to a corresponding heavy chain containing the VH regions from the parent antibodies. Methods for generating DVD-Ig molecules from two parent antibodies are further described in, e.g., PCT Publication Nos. WO 08/024188 and WO 07/024715, the disclosures of each of which are incorporated herein by reference in their entirety.

In some embodiments, an antibody, or antigen-binding fragment thereof, described herein can comprise an altered or variant Fc constant region (as discussed above), e.g., one which has reduced or no ADCC/CDC activity or increased affinity for FcRn.

In some embodiments, an antibody specifically binds to a protein of interest. The terms “specific binding,” “specifically binds,” and like grammatical terms, as used herein, refer to two molecules forming a complex that is relatively stable under physiologic conditions. Typically, binding is considered specific when the association constant (kₐ) is higher than 10⁶ M⁻¹s⁻¹. Thus, an antibody can specifically bind to a protein with a kₐ of at least (or greater than) 10⁶ (e.g., at least or greater than 10⁷, 10⁸, 10⁹, 10¹⁰, 10¹¹, 10¹², 10¹³, 10¹⁴, or 10¹⁵ or higher) M⁻¹s⁻¹. In some embodiments, an antibody described herein has a dissociation constant (k_{d}) of less than or equal to 10³ (e.g., 8×10⁻⁴, 5×10⁻⁴, 2×10⁻⁴, 10⁻⁴, or 10⁻⁵) s⁻¹.

In some embodiments, an antibody described herein has a K_{D} of less than 10⁻⁸, 10⁻⁹, 10⁻¹⁰, 10⁻¹¹, or 10⁻¹² M. The equilibrium constant KD is the ratio of the kinetic rate constants—k_{d}/kₐ. In some embodiments, an antibody described herein has a K_{D} of less than 1×10⁻⁹ M.

Methods for determining whether an antibody binds to a target antigen and/or the affinity for an antibody to a target antigen are known in the art. For example, the binding of an antibody to a protein antigen can be detected and/or quantified using a variety of techniques such as, but not limited to, Western blot, dot blot, plasmon surface resonance method (e.g., BIAcore system; Pharmacia Biosensor AB, Uppsala, Sweden and Piscataway, N.J.), or enzyme-linked immunosorbent assays (ELISA). See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2^{nd} Edition, Oxford University Press, NY, Oxford; Johne et al. (1993) J. Immunol. Meth. 160:191-198; Jonsson et al. (1993) Ann. Biol. Clin. 51:19-26; and Jonsson et al. (1991) Biotechniques 11:620-627. See also, U.S. Pat. No. 6,355,245.

The disclosure also features non-antibody, scaffold proteins that bind to modified ACC2 polypeptides (e.g., all of part of an ACC2 polypeptide comprising a modification of proline 450 relative to SEQ ID NO:2). These proteins are, generally, obtained through combinatorial chemistry-based adaptation of pre-existing antigen-binding proteins. For example, the binding site of human transferrin for human transferrin receptor can be modified using combinatorial chemistry to create a diverse library of transferrin variants, some of which have acquired affinity for different antigens. Ali et al. (1999) J Biol Chem 274:24066-24073. The portion of human transferrin not involved with bind the receptor remains unchanged and serves as a scaffold, like framework regions of antibodies, to present the variant binding sites. The libraries are then screened, as an antibody library is, against a target antigen of interest to identify those variants having optimal selectivity and affinity for the target antigen. Non-antibody scaffold proteins, while similar in function to antibodies, are touted as having a number of advantages as compared to antibodies, which advantages include, among other things, enhanced solubility and tissue penetration, less costly manufacture, and ease of conjugation to other molecules of interest. Hey et al. (2005) TRENDS Biotechnol 23(10):514-522.

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an akyrin repeat protein, a human lipocalin, human crystallin, human ubiquitin, or a trypsin inhibitor from E. elaterium. Id.

In some embodiments, an antibody or antigen-binding fragment thereof described herein is cross-reactive. The term “cross-reactive antibody,” as used herein, refers to an antibody capable of binding to a cross-reactive antigenic determinant. In some embodiments, an antibody or antigen-binding fragment thereof is cross-reactive for modified ACC2 polypeptides of different species. For example, an antibody described herein can bind to a human ACC2 containing a hydroxylated proline at position 450 relative to SEQ ID NO:2, as well as bind to a ACC2 protein from a non-human primate, such as Rhesus or Cynomolgus macaque, which also contains the hydroxylated proline residue. In some embodiments, an antibody or antigen-binding fragment thereof described herein can bind to a modified ACC2 polypeptide from human and rodent (e.g., mouse or rat) origin.

In some embodiments, the antibody preferentially binds to an ACC2 polypeptide when hydroxylated at proline 450 relative to SEQ ID NO:2 over the ACC2 polypeptide when not hydroxylated at proline 450 relative to SEQ ID NO:2. As used herein, “preferentially binding” is at least a 2 (e.g., at least 3, 4, 5, 6, 7, 8, 9, 10, 20, 30, 40, 50, 100, 200, 300, 400, 500, or 1000) fold greater affinity for an ACC2 polypeptide hydroxylated at proline 450 as compared to the affinity of the antibody for ACC2 that is not hydroxylated at proline 450.

In some embodiments, the antibody or antigen-binding fragment thereof binds to P450-hydroxylated ACC2 polypeptide with a K_{D} that is less than 2 nM. In some embodiments, the antibody or antigen-binding fragment thereof binds to P450-hydroxylated ACC2 polypeptide with a K_{D} that is less than 1 nM [also referred to herein as “subnanomolar affinity” ].

In some embodiments, the antibody or antigen-binding fragment thereof binds to P450-hydroxylated ACC2 polypeptide with a subnanomolar affinity [e.g., a K_{D} of less than or equal to 9.9×10⁻¹⁰ (e.g., less than or equal to 9×10⁻¹⁰, 8×10⁻¹⁰, 7×10⁻¹⁰, 6×10⁻¹⁰, 5×10⁻¹⁰, 4×10⁻¹⁰, 3×10⁻¹⁰, 2.5×10⁻¹⁰, 2×10⁻¹⁰, 1×10⁻¹⁰, 8.0×10⁻¹¹, 7.0×10⁻¹¹, 6.0×10⁻¹¹, 5.0×10⁻¹¹, 4.0×10⁻¹¹, or 3.0×10⁻¹¹) M] in the presence of a molar excess of ACC2 that is not hydroxylated at proline 450. In some embodiments, any of the antibodies or antigen-binding fragments thereof described herein have at least a 100 (e.g., at least 110, 120, 130, 140, 150, 160, 170, 180, 190, 200, 225, 250, 275, 300, 400, 500, 600, 700, 800, 900, 1000, 2000, 3000, 4000, 5000, 6000, 7000, 8000, 9000, or 10000)-fold greater affinity (e.g., represented by its K_{D}) for P450-hydroxylated ACC2 polypeptide than for unmodified ACC2.

In some embodiment, an antibody or antigen-binding fragment thereof: (a) binds to P450-hydroxylated ACC2 polypeptide with a subnanomolar affinity and (b) binds to P450-hydroxylated ACC2 polypeptide with an affinity that is at least 100 (e.g., at least 110, 120, 130, 140, 150, 160, 170, 180, 190, 200, 225, 250, 275, 300, 400, 500, 600, 700, 800, 900, 1000, 2000, 3000, 4000, 5000, 6000, 7000, 8000, 9000, or 10000)-fold greater than its corresponding affinity for unmodified ACC2. For example, an antibody or antigen-binding fragment thereof described herein can, in some embodiments, bind to P450-hydroxylated ACC2 polypeptide with a K_{D} of 100 nM and to at least a subpopulation of unmodified ACC2 polypeptide with a K_{D} that is at least 100-fold higher (e.g., at least 10 nM).

In some embodiment, the antibody or antigen-binding fragment thereof that binds to a ACC2 polypeptide having the amino acid sequence depicted in any one of SEQ ID NOs:2-9 in which the proline at position 450 is hydroxylated, wherein the antibody or antigen-binding fragment thereof binds to the P450-hydroxylated ACC2 polypeptide with a K_{D} that is less than 1.25×10⁻⁹ M in the presence of a molar excess (e.g., a 2, 3, 4, 5, 6, 7, 8, 9, 10, 15, 20, 25, 30, 35, 40, 45, 50, 60, 70, 80, 90, 100, 150, 200, 300, 400, or 500-fold molar excess) of unmodified ACC2 polypeptide. In some embodiments, the antibody or antigen-binding fragment thereof binds to a P450-hydroxylated ACC2 polypeptide with a subnanomolar affinity (e.g., any of the subnanomolar K_{D}'s recited herein) in the presence of at least, or greater than, a 2-fold molar excess, but no greater or less than a 500 (e.g., 500, 450, 400, 350, 300, 250, 200, 150, 100, 90, 80, 70, 60, 50, 40, 30, 25, 20, or 15)-fold molar excess of unmodified ACC2 polypeptide over P450-hydroxylated ACC2 polypeptide. Such measurements can be in vitro measurements using, e.g., standard affinity determination techniques, many of which are recited and/or described herein.

In some embodiments, the isolated antibody, or fragment thereof, only binds to an ACC2 polypeptide when hydroxylated at proline 450 relative to SEQ ID NO:2 (e.g., no detectable binding of the antibody to unmodified ACC2 above background levels observed with a control antibody).

In some embodiments, the isolated antibody, or fragment thereof, that specifically binds to an ACC2 polypeptide that is hydroxylated at proline 450 relative to SEQ ID NO:2, wherein the antibody specifically binds to an epitope that is within any one of the amino acid sequences depicted in SEQ ID NOs: 2-9.

In some embodiments, the antibody preferentially binds to an ACC2 polypeptide when hydroxylated at proline 343, 450, and/or 2131 relative to SEQ ID NO:2 over the ACC2 polypeptide when not hydroxylated at proline 343, 450, and/or 2131 relative to SEQ ID NO:2. As used herein, “preferentially binding” is at least a 2 (e.g., at least 3, 4, 5, 6, 7, 8, 9, 10, 20, 30, 40, 50, 100, 200, 300, 400, 500, or 1000) fold greater affinity for an ACC2 polypeptide hydroxylated at proline 343, 450, and/or 2131 as compared to the affinity of the antibody for ACC2 that is not hydroxylated at proline 343, 450, and/or 2131.

In some embodiments, the antibody or antigen-binding fragment thereof binds to hydroxylated ACC2 polypeptide with a K_{D} that is less than 2 nM. In some embodiments, the antibody or antigen-binding fragment thereof binds to P450-hydroxylated ACC2 polypeptide with a K_{D} that is less than 1 nM.

In some embodiments, the antibody or antigen-binding fragment thereof binds to hydroxylated ACC2 polypeptide with a subnanomolar affinity [e.g., a K_{D} of less than or equal to 9.9×10⁻¹⁰ (e.g., less than or equal to 9×10⁻¹⁰, 8×10⁻¹⁰, 7×10⁻¹⁰, 6×10⁻¹⁰, 5×10⁻¹⁰, 4×10⁻¹⁰, 3×10⁻¹⁰, 2.5×10⁻¹⁰, 2×10⁻¹⁰, 1×10⁻¹⁰, 8.0×10⁻¹¹, 7.0×10⁻¹¹, 6.0×10⁻¹¹, 5.0×10⁻¹¹, 4.0×10⁻¹¹, or 3.0×10⁻¹¹) M] in the presence of a molar excess of ACC2 that is not hydroxylated at proline 343, 450, and/or 2131. In some embodiments, any of the antibodies or antigen-binding fragments thereof described herein have at least a 100 (e.g., at least 110, 120, 130, 140, 150, 160, 170, 180, 190, 200, 225, 250, 275, 300, 400, 500, 600, 700, 800, 900, 1000, 2000, 3000, 4000, 5000, 6000, 7000, 8000, 9000, or 10000)-fold greater affinity (e.g., represented by its K_{D}) for hydroxylated ACC2 polypeptide than for unmodified ACC2.

In some embodiment, an antibody or antigen-binding fragment thereof: (a) binds to hydroxylated ACC2 polypeptide with a subnanomolar affinity and (b) binds to hydroxylated ACC2 polypeptide with an affinity that is at least 100 (e.g., at least 110, 120, 130, 140, 150, 160, 170, 180, 190, 200, 225, 250, 275, 300, 400, 500, 600, 700, 800, 900, 1000, 2000, 3000, 4000, 5000, 6000, 7000, 8000, 9000, or 10000)-fold greater than its corresponding affinity for unmodified ACC2. For example, an antibody or antigen-binding fragment thereof described herein can, in some embodiments, bind to hydroxylated ACC2 polypeptide with a K_{D} of 100 nM and to at least a subpopulation of unmodified ACC2 polypeptide with a K_{D} that is at least 100-fold higher (e.g., at least 10 nM).

In some embodiment, the antibody or antigen-binding fragment thereof that binds to a ACC2 polypeptide having the amino acid sequence depicted in any one of SEQ ID NOs:2-5 in which the proline at position 343, 450, and/or 2131 is hydroxylated, wherein the antibody or antigen-binding fragment thereof binds to the hydroxylated ACC2 polypeptide with a K_{D} that is less than 1.25×10⁻⁹ M in the presence of a molar excess (e.g., a 2, 3, 4, 5, 6, 7, 8, 9, 10, 15, 20, 25, 30, 35, 40, 45, 50, 60, 70, 80, 90, 100, 150, 200, 300, 400, or 500-fold molar excess) of unmodified ACC2 polypeptide. In some embodiments, the antibody or antigen-binding fragment thereof binds to a hydroxylated ACC2 polypeptide with a subnanomolar affinity (e.g., any of the subnanomolar K_{D}'s recited herein) in the presence of at least, or greater than, a 2-fold molar excess, but no greater or less than a 500 (e.g., 500, 450, 400, 350, 300, 250, 200, 150, 100, 90, 80, 70, 60, 50, 40, 30, 25, 20, or 15)-fold molar excess of unmodified ACC2 polypeptide over hydroxylated ACC2 polypeptide. Such measurements can be in vitro measurements using, e.g., standard affinity determination techniques, many of which are recited and/or described herein.

In some embodiments, the isolated antibody, or fragment thereof, only binds to an ACC2 polypeptide when hydroxylated at proline 343, 450, and/or 2131 relative to SEQ ID NO:2 (e.g., no detectable binding of the antibody to unmodified ACC2 above background levels observed with a control antibody).

In some embodiments, the isolated antibody, or fragment thereof, only binds to an ACC2 polypeptide (or preferentially binds to an ACC2 polypeptide) when not hydroxylated at proline 343, 450, and/or 2131 relative to SEQ ID NO:2 (e.g., no detectable binding of the antibody to modified ACC2 above background levels observed with a control antibody).

### Diagnostic Methods

As noted above, the instant disclosure provides the discovery that prolyl hydroxylase 3 (PHD3) specifically hydroxylates acetyl-CoA carboxylase 2 (ACC2) at position 450 (relative to SEQ ID NO:2). PHD3-dependent hydroxylation enhances the activity of ACC2, the result of which is reduced fatty acid oxidation (FAO). Also discovered was that cancer cells with lower levels of PHD3 expression are more sensitive to FAO inhibitors; conversely, cancer cells with higher levels of PHD3 expression, and thus lower levels of FAO, are more reliant on glycolysis and thus more sensitive to glycolytic pathway inhibitors. Accordingly, detecting or monitoring the level of PHD3 expression or ACC2 hydroxylation is useful for a number of diagnostic and therapeutic indications, such as the following.

The disclosure also provides the discovery PHD3 can hydroxylate ACC2 at positions 343 and 2131 (relative to SEQ ID NO:2).

Methods for detecting or measuring the expression level of a protein, or mRNA encoding the protein, in a biological sample are well known in the art, the specification exemplifies methods for detecting the expression of PHD3. For example, mRNA expression can be determined using Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res 6(10):995-1001; and Zhang et al. (2005) Environ Sci Technol 39(8):2777-2785; U.S. Patent Application Publication No. 2004086915; European Patent No. 0543942; and U.S. Pat. No. 7,101,663; the disclosures of each of which are incorporated herein by reference in their entirety.

In one example, the presence or amount of one or more discrete mRNA populations in a biological sample can be determined by isolating total mRNA from the biological sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled polynucleotide probes, complementary to the mRNA sequence of interest, which bind to and thus render detectable their corresponding mRNA populations. Detectable labels include, e.g., fluorescent (e.g., fluorescein, fluorescein isothiocyanate, rhodamine, dichlorotriazinylamine fluorescein, dansyl chloride, allophycocyanin (APC), or phycoerythrin), luminescent (e.g., europium, terbium, Qdot™ nanoparticles supplied by the Quantum Dot Corporation, Palo Alto, Calif.), radiological (e.g., 125I, 131I, 35S, 32P, 33P, or 3H), and enzymatic (horseradish peroxidase, alkaline phosphatase, beta-galactosidase, or acetylcholinesterase) labels.

In another example, the presence or amount of discrete populations of mRNA in a biological sample can be determined using nucleic acid (or oligonucleotide) arrays. For example, isolated mRNA from a biological sample can be amplified using RT-PCR with random hexamer or oligo(dT)-primer mediated first strand synthesis. The RT-PCR step can be used to detectably-label the amplicons, or, optionally, the amplicons can be detectably labeled subsequent to the RT-PCR step. For example, the detectable label can be enzymatically (e.g., by nick translation or a kinase such as T4 polynucleotide kinase) or chemically conjugated to the amplicons using any of a variety of suitable techniques (see, e.g., Sambrook et al., supra). The detectably-labeled amplicons are then contacted to a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence or amount of a target mRNA in the biological sample. Additional methods for detecting mRNA expression using nucleic acid arrays are described in, e.g., U.S. Pat. Nos. 5,445,934; 6,027,880; 6,057,100; 6,156,501; 6,261,776; and 6,576,424; the disclosures of each of which are incorporated herein by reference in their entirety.

Methods of detecting and/or for quantifying a detectable label depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

RNA can be extracted from the tissue sample by a variety of methods, e.g., the guanidium thiocyanate lysis followed by CsCl centrifugation (Chirgwin et al. 1979, Biochemistry 18:5294-5299). RNA from single cells can be obtained as described in methods for preparing cDNA libraries from single cells, such as those described in Dulac (1998) Curr Top Dev Biol 36:245 and Jena et al. (1996) J Immunol Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc Natl Acad Sci USA 86:9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by Marshall et al., (1994) PCR Methods and Applications 4: 80-84. Real time PCR may also be used.

Other known amplification methods which can be utilized herein include but are not limited to the so-called “NASBA” or “3 SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace (1989) Genomics 4: 560; Landegren et al. (1988) Science 241:1077); self-sustained sequence replication (SSR) (see, e.g., Guatelli et al. (1990) Proc Nat Acad Sci USA 87:1874); and transcription amplification (see, e.g., Kwoh et al. (1989) Proc Natl Acad Sci USA 86:1173).

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

In other embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

The expression of a protein can also be determined by detecting and/or measuring expression of a protein. Methods of determining protein expression generally involve the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan et al., eds. (1995) Current Protocols in Immunology. Wiley, New York), or antibody array analysis (see, e.g., U.S. Patent Application Publication Nos. 20030013208 and 2004171068, the disclosures of each of which are incorporated herein by reference in their entirety). Further description of many of the methods above and additional methods for detecting protein expression can be found in, e.g., Sambrook et al. (supra).

In one example, the presence or amount of protein expression can be determined using a western blotting technique. For example, a lysate can be prepared from a biological sample, or the biological sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In another example, an immunoassay can be used for detecting and/or measuring the protein expression of a protein. As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here as above, the presence or amount of bound detectably-labeled antibody indicates the presence or amount of protein in the biological sample.

Methods for generating antibodies or antibody fragments specific for a protein can be generated by immunization, e.g., using an animal, or by in vitro methods such as phage display (see above under the section titled “Antibodies”). A polypeptide that includes all or part of a target protein can be used to generate an antibody or antibody fragment. The antibody can be a monoclonal antibody or a preparation of polyclonal antibodies.

Methods for detecting or measuring gene expression can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

The phrase “elevated level of expression” is used interchangeably with “overexpression” and means an increase in the expression level of protein or nucleic acid molecule, relative to a control level. For example, a putative cancer cell may overexpress a protein (e.g., PHD3) relative to a normal cell of the same histological type from which the cancer cell evolved. Overexpression includes an increased expression of a given gene, relative to a control level, of at least 5 (e.g., at least 10, 15, 20, 25, 30, 35, 40, 45, 50, 55, 60, 65, 70, 75, 80, 85, 90, 95, 100, 110, 120, 130 140 150, 160 170, 180, 190, 200, or more) %. Overexpression includes an increased expression, relative to a control level, of at least 1.5 (e.g., at least 2, 2.5, 3, 4, 5, 6, 7, 8, 9, 10, 20, 30, 40, 50, 100, 1000 or more) fold.

Conversely, the phrase “reduced level of expression” or like grammatical phrases means an decrease in the expression level of protein or nucleic acid molecule, relative to a control level. For example, a putative cancer cell may have reduced expression of a protein (e.g., PHD3) relative to a normal cell of the same histological type from which the cancer cell evolved. In some embodiments, the level of mRNA or protein expression by a cell of interest (e.g., a cancer cell) is less than or equal to 99 (e.g., less than or equal to 98, 97, 96, 95, 94, 93, 92, 91, 90, 85, 80, 75, 70, 65, 60, 55, 50, 45, 40, 35, 30, 25, 20, 15, 10, 9, 8, 7, 6, or 5) % of a control level, e.g., the level in normal cells of the same histological type.

Also featured are methods for detecting the presence of an ACC2 polypeptide, or portion thereof, comprising a modification at proline 450 relative to SEQ ID NO:2 (e.g., a hydroxylated proline 450) in a biological sample. The biological sample can be, e.g., cells (e.g., cancer cells) or a lysate prepared from such cells. The method includes: (a) contacting the biological sample with a detection reagent under conditions suitable for formation of a complex between the detection reagent and ACC2 that is hydroxylated at proline 450 relative to SEQ ID NO:2, if such hydroxylated ACC2 is present in the biological sample; and (b) detecting the presence or amount of the detection reagent as a measure of the presence or amount of the complex in the biological sample, wherein the presence of the complex indicates the presence of hydroxylated ACC2 in the biological sample. In some embodiments, the detection reagent is an antibody or non-antibody scaffold protein that binds to ACC2 when hydroxylated at position 450 relative to SEQ ID NO:2. The antibody or non-antibody scaffold protein can be, e.g., any of those described herein. In some embodiments, the antibody or non-antibody scaffold protein is detectably-labeled, e.g., with an enzymatic label, a radioactive label, or a fluorescent label.

In some embodiments, the methods include: (a) contacting the biological sample with at least one antibody (or non-antibody scaffold protein) under conditions suitable for formation of a complex between the antibody and ACC2 that is hydroxylated at proline 450 relative to SEQ ID NO:2, if such hydroxylated ACC2 is present in the biological sample; and (b) detecting the presence of the complex in the biological sample, wherein the presence of the complex indicates the presence of hydroxylated ACC2 in the biological sample.

In some embodiments, the methods include: (a) contacting a biological sample with at least one antibody (or non-antibody scaffold protein) under conditions suitable for formation of a complex between the antibody and ACC2 that is hydroxylated at proline 450 relative to SEQ ID NO:2, if such hydroxylated ACC2 is present in the biological sample; (b) contacting the complex of (a) with a detection reagent; and (c) detecting the presence or amount of the detection reagent as a measure of the presence or amount of the complex in the biological sample, wherein the presence of the complex indicates the presence of P450-hydroxylated ACC2 in the biological sample. In some embodiments, the detection reagent is a binding agent that specifically binds to the antibody or non-antibody scaffold protein of the complex. In some embodiments, e.g., where the antibody or non-antibody scaffold protein comprises a first member of a specific binding pair (streptavidin or biotin), the detection reagent can be a detectably-labeled second member of the binding pair.

Methods for detecting or quantifying a detection agent are known in the art. For example, an antibody-ACC2 complex can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass spectrometry based methods. A variety of immunoassay techniques, including competitive and non-competitive immunoassays, can be used. The term “immunoassay” encompasses techniques including, without limitation, flow cytometry, FACS, enzyme immunoassays (EIA), such as enzyme multiplied immunoassay technique (EMIT), enzyme-linked immunosorbent assay (ELISA), IgM antibody capture ELISA (MAC ELISA) and microparticle enzyme immunoassay (MEIA), furthermore capillary electrophoresis immunoassays (CEIA), radio-immunoassays (RIA), immunohistochemistry, immunoradiometric assays (IRMA), fluorescence polarization immunoassays (FPIA) and chemiluminescence assays (CL). If desired, such immunoassays can be automated.

Immunoassays can also be used in conjunction with laser induced fluorescence. Liposome immunoassays, such as flow-injection liposome immunoassays and liposome immunosensors, are also suitable for use in the present invention. In addition, nephelometry assays, in which, for example, the formation of protein/antibody complexes results in increased light scatter that is converted to a peak rate signal as a function of the marker concentration, are suitable for use in the methods of the present invention. In a preferred embodiment of the present invention, the incubation products are detected by ELISA, RIA, fluoro immunoassay (FIA) or soluble particle immune assay (SPIA).

In some embodiments, a reduced level of ACC2 hydroxylated at proline 450 by cancer cells of a subject's cancer, relative to a control level, indicates that the cancer cells are susceptible to a fatty acid oxidation inhibitor. In some embodiments, an elevated level of ACC2 hydroxylated at proline 450, relative to a control level, indicates that the cancer is susceptible to a glycolytic pathway inhibitor.

In some embodiments, a reduced level of PHD3 expression by cancer cells of a subject's cancer, relative to a control level, indicates that the cancer cells are susceptible to a fatty acid oxidation inhibitor. In some embodiments, an elevated level of PHD3 expression, relative to a control level, indicates that the cancer is susceptible to a glycolytic pathway inhibitor.

The term “control” refers to any reference standard suitable to provide a comparison to the test sample. As described above, the methods described herein can involve comparing the expression level of PHD3 and/or the level of hydroxylation of ACC2 to a control amount. In some embodiments, the control is a control sample obtained from a normal, healthy subject of the same species who does not have, is not suspected of having, and/or is not at risk for developing a cancer. For example, the control can be the expression level or level of hydroxylated ACC2 found in normal cells of the same histological type from which the cancer evolved and from the same species as the subject. In some embodiments, the control can be (or can be based on), e.g., a collection of samples obtained from two or more (e.g., two, three, four, five, six, seven, eight, nine, 10, 15, 20, 25, 30, 35, or 40 or more) healthy individuals (e.g., a mean or median level). In some embodiments, the control can be (or can be based on), e.g., one sample or a collection of samples obtained from two or more (e.g., two, three, four, five, six, seven, eight, nine, 10, 15, 20, 25, 30, 35, or 40 or more) individuals (e.g., a mean or median level) determined to be in clinical remission of an autoimmune disease (e.g., MS). In some embodiments, the control amount is detected or measured concurrently with the test sample. In some embodiments, the control level or amount is a pre-determined range or threshold based on, e.g., average levels from a control group (e.g., normal healthy volunteer subjects). Thus, a normal control PHD3 expression level in a prostate cancer can be the expression level determined from cells of a prostate obtained from a healthy subject of the same species. A normal control expression level or level of hydroxylated ACC2 can be the mean, or a range of values around the mean, of obtained from measurements from two or more normal healthy subjects of the same species as the subject of interest. In some embodiments, the normal control expression level or level of hydroxylated ACC2 is a threshold value (e.g., determined based on the average levels from subjects with a particular cancer or a particular form of cancer, above or below which is indicative of a certain phenotype, e.g., sensitivity to an FAO inhibitor or a glycolytic pathway inhibitor.

In some embodiments, the control is a control sample obtained from a subject of the same species who has, is suspected of having, and/or is at risk for developing a cancer of the same type as that of the subject. In some embodiments, the control can be (or can be based on), e.g., a collection of samples obtained from two or more (e.g., two, three, four, five, six, seven, eight, nine, 10, 15, 20, 25, 30, 35, or 40 or more) individuals of the same species (e.g., a mean or median level) who have a cancer of the same type.

As demonstrated by the data below, the methods of the present invention are not limited to use of a specific cut-point in comparing a level of expression of PHD3 or level of hydroxylated ACC2 polypeptide in the test sample to the control.

### Kits

A “kit” is any manufacture (e.g., a package or container) comprising at least one reagent described herein, e.g., one or more of the polypeptides, antibodies, non-antibody scaffold proteins, vectors, expression vectors, cells, or detection reagents provided herein, e.g., useful in diagnostic, research, and/or therapeutic applications, such as determining PHD3 expression levels by cells, the level of modified ACC2 in cells, or whether a cancer cell is sensitive to a glycolytic pathway inhibitor or a FAO inhibitor. The kit may be promoted, distributed, or sold as a unit for performing the methods of the present disclosure. In certain embodiments, the kit may further comprise a reference standard (normal cells or lysate of normal cells) and/or one or more suitable buffers. In addition, instructional materials which describe the use of the compositions within the kit can be included.

In some embodiments, the kit comprises a means for obtaining a biological sample from a subject (e.g., a syringe).

### Test Compounds and Methods for Screening

The disclosure also feature methods for identifying a modulator of PHD3 activity (or methods for identifying a modulator of P343, P450, or P2131 hydroxylation of ACC2). The methods can include: contacting, in the presence of all or part of an ACC2 polypeptide that contains the proline at position 450 relative to SEQ ID NO:2 (also referred to herein as a substrate ACC2 protein), a PHD3 protein or an enzymatically-active fragment thereof with a candidate compound; and detecting hydroxylation of the substrate ACC2 protein by the PHD3 protein or enzymatically-active fragment thereof. A difference in the amount of hydroxylation of the substrate ACC2 protein by the PHD3 protein or enzymatically-active fragment thereof in the presence of the candidate compound, as compared to the amount of hydroxylation of the substrate ACC2 protein by the PHD3 protein or enzymatically-active fragment thereof in the absence of the candidate compound, indicates that the candidate compound modulates PHD3 activity. In some embodiments, the candidate compound inhibits the hydroxylation by PHD3 of the substrate ACC2 protein. In some embodiments, the candidate compounds enhances the hydroxylation by PHD3 of substrate ACC2 protein.

In some embodiments, the substrate ACC2 protein comprises or consists of the amino acid sequence depicted in any one of SEQ ID NOs: 2-9.

In some embodiments, the methods can include: contacting, in the presence of all or part of an ACC2 polypeptide that contains the proline at positions 343, 450, and 2131 relative to SEQ ID NO:2, a PHD3 protein or an enzymatically-active fragment thereof with a candidate compound, and detecting hydroxylation of the substrate ACC2 protein by the PHD3 protein or enzymatically-active fragment thereof. A difference in the amount of hydroxylation of the substrate ACC2 protein by the PHD3 protein or enzymatically-active fragment thereof in the presence of the candidate compound, as compared to the amount of hydroxylation of the substrate ACC2 protein by the PHD3 protein or enzymatically-active fragment thereof in the absence of the candidate compound, indicates that the candidate compound modulates PHD3 activity. In some embodiments, the candidate compound inhibits the hydroxylation by PHD3 of the substrate ACC2 protein. In some embodiments, the candidate compounds enhances the hydroxylation by PHD3 of substrate ACC2 protein.

As used herein, a PHD3 protein includes wild-type PHD3 polypeptides from any species (e.g., human, rodent, or non-human primate origin) as well as variants of such polypeptides containing amino acid insertions, deletions, or substitutions (e.g., conservative or non-conservative substitutions). The PHD3 polypeptides, including variants and enzymatically-active fragments of PHD3 polypeptides or variants, retain at least 5 (e.g., at least 10, 15, 20, 25, 30, 35, 40, 45, 50, 55, 60, 65, 70, 75, 80, 85, 90, 95, or 100) % of the ability of the corresponding full-length, wild-type PHD3 polypeptide from which the variant or fragment was derived to hydroxylate ACC2 at proline 450 relative to SEQ ID NO:2. In vitro hydroxylation methods are described herein and exemplified in the working examples. An exemplary amino acid sequence for human a PHD3 polypeptide is as follows (SEQ ID NO: 1):

| | |
|---|---|
| 1 | mplghimrld lekialeyiv pclhevgfcy ldnflgevvg dcvlervkql hctgalrdgq |
| 61 | lagpragvsk rhlrgdqitw iggneegcea isfllslidr lvlycgsrlg kyyvkerska |
| 121 | mvacypgngt gyvrhvdnpn gdgrcitciy ylnknwdakl hggilrifpe gksfiadvep |
| 181 | ifdrllffws drrnphevqp syatryamtv wyfdaeerae akkkfrnltr ktesalted |

In vivo hydroxylation assays are known in the art and exemplified herein. For example, a cell can be transfected with one or more expression vectors encoding one or both of a PHD3 polypeptide (or variant or biologically-active fragment thereof) and a substrate ACC2 polypeptide. The cells expressing the proteins can be cultured in the presence or absence of a test compound. The cells can optionally be cultured under a stress condition (e.g., hypoxia, low sugar conditions, or in the presence of citrate) that stimulates hydroxylation of ACC2 by PHD3. The presence or amount of P450-hydroxylated substrate ACC2 protein can be measured in situ, e.g., by immunohistochemistry and/or FACS (see above). Alternatively, lysates can be prepared from cells and subjected to, e.g., Western blotting, dot blotting, or the like to determine the presence or amount of hydroxylated substrate ACC2 protein.

In some embodiments, cells for use in the methods described herein express PHD3 and ACC2 in amounts suitable to detect the presence or amount of a change in P450-hydroxylation of ACC2 in the presence of a test compound, e.g., under a stress condition.

A test compound described herein can be, e.g., a small molecule, a protein, a protein fragment, a polypeptide, a peptide, a polypeptide analog, a peptidomimetic, a nucleic acid, a nucleic acid analog, a macrocyle compound, an aptamer including but not limited to an RNA aptamer including an L-RNA aptamer, a spiegelmer, a locked nucleic acid (LNA), a peptide nucleic acid (PNA), or an antibody. In some embodiments, the small molecule can be a non-antibody antigen-binding protein, e.g., one of the antibody-related scaffold protein constructs as described in Hey et al. (2005) TRENDS in Biotechnology 23(1):514-522.

In some embodiments, the candidate or test compound binds to PHD3 or ACC2. Methods for determining whether a compound binds to a target protein, such as PHD3 or ACC2, and/or the affinity for an agent for a target protein are known in the art. For example, the binding of an agent to a target protein can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass spectrometry based methods. In situ methods for detecting PHD3-dependent hydroxylation of

In some embodiments, binding of test compounds to a PHD3 or ACC2 polypeptide can be assayed using thermodenaturation methods involving differential scanning fluorimetry (DSF) and differential static light scattering (DSLS).

In some embodiments, binding of test compounds to a PHD3 or ACC2 polypeptide can be assayed using a mass spectrometry based method such as, but not limited to, an affinity selection coupled to mass spectrometry (AS-MS) platform. This is a label-free method where the protein and test compound are incubated, unbound molecules are washed away and protein-ligand complexes are analyzed by MS for ligand identification following a decomplexation step.

In some embodiments, binding of test compounds to a PHD3 or ACC2 polypeptide can be quantitated using, for example, detectably labeled proteins such as radiolabeled (e.g., ³²P, ³⁵S, ¹⁴C or ³H), fluorescently labeled (e.g., FITC), or enzymatically labeled polypeptide or test compound, by immunoassay, or by chromatographic detection.

In some embodiments, the present invention contemplates the use of fluorescence polarization assays and fluorescence resonance energy transfer (FRET) assays in measuring, either directly or indirectly, the degree of interaction between a polypeptide and a test compound.

All of the above embodiments are suitable for development into high-throughput platforms.

In some embodiments, a compound that is determined to bind to PHD3 and/or inhibit PHD3-dependent hydroxylation of ACC2 can be further evaluated for its biological effect in cells. For example, the compound can be screened for its ability to inhibit ACC2 activity in a cell. As described above, ACC2 catalyzes the carboxylation of acetyl-CoA to malonyl-CoA. Methods for measuring the enzymatic activity of ACC2 are known in the art and exemplified in the working examples. In some embodiments, other indicia of FAO are measured.

Thus, in some embodiments, cells (e.g., comprising expression vectors encoding one or both of PHD3 and ACC2) are cultured in the presence or absence of the compound for a time sufficient to allow conversion of acetyl-CoA to malonyl-CoA by ACC2 in the absence of the compound. A difference in the amount of malonyl-CoA produced in the presence of the candidate compound, as compared to the amount of malonyl-CoA produced in the absence of the candidate compound, indicates that the candidate compound modulates the activity of ACC2. In some embodiments, the candidate compound inhibits the production of malonyl-CoA. In some embodiments, the candidate compounds enhances the production of malonyl-CoA.

### Inhibitors

As used herein, “inhibition” or the action of an “inhibitor” of a gene or gene product (e.g., PHD3) can be inhibition of: (i) the transcription of a coding sequence for one of the gene products, (ii) the translation of an mRNA encoding one of the gene products, (iii) the stability of an mRNA encoding one of the gene products, (iv) the intracellular trafficking of one of the gene products, (v) the stability of the gene products (i.e., protein stability or turnover), (vi) the interaction of the gene product with another protein (e.g., inhibition of the interaction between PHD3 and ACC2), and/or (vii) the activity of one of the gene products (e.g., inhibition of the enzymatic activity of PHD3). The compound can be, e.g., a small molecule, a nucleic acid or nucleic acid analog, a peptidomimetic, a polypeptide, a macrocycle compound, or a macromolecule that is not a nucleic acid or a protein. These compounds include, but are not limited to, small organic molecules, RNA aptamers, L-RNA aptamers, Spiegelmers, nucleobase, nucleoside, nucleotide, antisense compounds, double stranded RNA, small interfering RNA (siRNA), locked nucleic acid inhibitors, peptide nucleic acid inhibitors, and/or analogs of any of the foregoing. In some embodiments, a compound may be a protein or protein fragment.

As used herein, the term “inhibiting” and grammatical equivalents thereof refer to a decrease, limiting, and/or blocking of a particular action, function, or interaction. In one embodiment, the term refers to reducing the level of a given output or parameter to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The disclosure does not require, and is not limited to, methods that wholly eliminate the output or parameter.

As used herein, the term “interaction”, when referring to an interaction between two molecules, refers to the physical contact (e.g., binding) of the molecules with one another. Generally, such an interaction results in an activity (which produces a biological effect) of one or both of said molecules. To inhibit such an interaction results in the disruption of the activity of one or more molecules involved in the interaction.

Small Molecules and Peptides

“Small molecule” as used herein, is meant to refer to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc (1998) 120:8565-8566). It is within the scope of this application that such a library may be used to screen for inhibitors (e.g., hydroxylase inhibitors, kinase inhibitors) of any one of the gene products described herein, e.g., cyclin dependent kinases. There are numerous commercially available compound libraries, such as the Chembridge DIVERSet. Libraries are also available from academic investigators, such as the Diversity set from the NCI developmental therapeutics program. Rational drug design may also be employed.

Compounds useful in the methods of the present invention may be obtained from any available source, including systematic libraries of natural and/or synthetic compounds. Compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85, which is expressly incorporated by reference); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145, which is expressly incorporated by reference).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, each of which is expressly incorporated by reference.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra., each of which is expressly incorporated by reference).

Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of humans or animals. It should be noted that peptidomimetics may or may not have similar two-dimensional chemical structures, but share common three-dimensional structural features and geometry. Each peptidomimetic may further have one or more unique additional binding elements.

Nucleic Acids

Nucleic acid inhibitors can be used to decrease expression of an endogenous gene encoding one of the gene products described herein. The nucleic acid antagonist can be, e.g., an siRNA, a dsRNA, a ribozyme, a triple-helix former, an aptamer, or an antisense nucleic acid. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. The siRNA sequences can be, in some embodiments, exactly complementary to the target mRNA. dsRNAs and siRNAs in particular can be used to silence gene expression in mammalian cells (e.g., human cells). See, e.g., Clemens et al. (2000) Proc Natl Acad Sci USA 97:6499-6503; Billy et al. (2001) Proc Natl Acad Sci USA 98:14428-14433; Elbashir et al. (2001) Nature 411:494-8; Yang et al. (2002) Proc Natl Acad Sci USA 99:9942-9947, and U.S. Patent Application Publication Nos. 20030166282, 20030143204, 20040038278, and 20030224432. Antisense agents can include, for example, from about 8 to about 80 nucleobases (i.e. from about 8 to about 80 nucleotides), e.g., about 8 to about 50 nucleobases, or about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

siRNA molecules can be prepared by chemical synthesis, in vitro transcription, or digestion of long dsRNA by Rnase III or Dicer. These can be introduced into cells by transfection, electroporation, intracellular infection or other methods known in the art. See, for example, each of which is expressly incorporated by reference: Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Cur. Open. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9); 6047-6052, PCT publications WO2006/066048 and WO2009/029688, U.S. published application U.S. 2009/0123426, each of which is incorporated by reference in its entirety.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. Exemplary antisense compounds include DNA or RNA sequences that specifically hybridize to the target nucleic acid, e.g., the mRNA encoding one of the gene products described herein. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C₅-propynyl pyrimidines such as C₅-propynylcytosine and C₅-propynyluracil. Other suitable modified nucleobases include, e.g., 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. See, e.g., U.S. Pat. Nos. 4,987,071; 5,116,742; and U.S. Pat. No. 5,093,246; “Antisense RNA and DNA,” D. A. Melton, Ed., Cold Spring Harbor Laboratory, Cold Spring Harbor, N.Y. (1988); Haselhoff and Gerlach (1988) Nature 334:585-59; Helene, C. (1991) Anticancer Drug D 6:569-84; Helene (1992) Ann NY Acad Sci 660:27-36; and Maher (1992) Bioassays 14:807-15.

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol 173:305-326).

Antisense or RNA interference molecules can be delivered in vitro to cells or in vivo. Typical delivery means known in the art can be used. Any mode of delivery can be used without limitation, including: intravenous, intramuscular, intraperitoneal, intraarterial, local delivery during surgery, endoscopic, or subcutaneous. Vectors can be selected for desirable properties for any particular application. Vectors can be viral, bacterial or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g. plasmid, viral and bacterial vectors. Any nucleic acid delivery method known in the art can be used in the present invention. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In an embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53, which is expressly incorporated by reference. In addition, the reduced uptake by the RES lowers the toxicity of stealth liposomes by preventing significant accumulation of the liposomes in the liver and spleen.

The nucleotide sequences encoding the gene products described herein (from multiple species, including human), from which exemplary nucleic acid inhibitors can be designed, are known in the art and are publicly available. For example, an exemplary nucleotide sequence encoding human PHD3 is as follows:

| | |
|---|---|
| 1 | atgcccctgg gacacatcat gaggctggac ctggagaaaa ttgccctgga gtacatcgtg |
| 61 | ccctgtctgc acgaggtggg cttctgctac ctggacaact tcctgggcga ggtggtgggc |
| 121 | gactgcgtcc tggagcgcgt caagcagctg cactgcaccg gggccctgcg ggacggccag |
| 181 | ctggcggggc cgcgcgccgg cgtctccaag cgacacctgc ggggcgacca gatcacgtgg |
| 241 | atcgggggca acgaggaggg ctgcgaggcc atcagcttcc tcctgtccct catcgacagg |
| 301 | ctggtcctct actgcgggag ccggctgggc aaatactacg tcaaggagag gtctaaggca |
| 361 | atggtggctt gctatccggg aaatggaaca ggttatgttc gccacgtgga caaccccaac |
| 421 | ggtgatggtc gctgcatcac ctgcatctac tatctgaaca agaattggga tgccaagcta |
| 481 | catggtggga tcctgcggat atttccagag gggaaatcat tcatagcaga tgtggagccc |
| 541 | atttttgaca gactcctgtt cttctggtca gatcgtagga acccacacga agtgcagccc |
| 601 | tcttacgcaa ccagatatgc tatgactgtc tggtactttg atgctgaaga aagggcagaa |
| 661 | gccaaaaaga aattcaggaa tttaactagg aaaactgaat ctgccctcac tgaagactga |

(SEQ ID NO:13; NCBI reference no. NM_022073). In some embodiments, the siRNA is selective for PHD3 over other PHD forms, e.g., PHD1 and/or PHD2.

Antibodies

Although antibodies are most often used to inhibit the activity of extracellular proteins (e.g., receptors and/or ligands), the use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al., each of which is expressly incorporated by reference). Therefore, antibodies specific for any of the gene products described herein are useful as biological agents for the methods of the present invention.

### Biological Samples and Sample Collection

Suitable biological samples for use in the methods described herein include, e.g., any biological fluid. A biological sample can be, for example, a specimen obtained from a subject (e.g., a mammal such as a human) or can be derived from such a subject. A biological sample can also be a biological fluid such as urine, whole blood or a fraction thereof (e.g., plasma or serum), saliva, semen, sputum, cerebrospinal fluid, tears, or mucus. A biological sample can be further fractionated, if desired, to a fraction containing particular analytes (e.g., proteins) of interest. For example, a whole blood sample can be fractionated into serum or into fractions containing particular types of proteins. If desired, a biological sample can be a combination of different biological samples from a subject such as a combination of two different fluids.

Biological samples suitable for the invention may be fresh or frozen samples collected from a subject, or archival samples with known diagnosis, treatment and/or outcome history. The biological samples can be obtained from a subject, e.g., a subject having, suspected of having, or at risk of developing, a cancer. Any suitable methods for obtaining the biological samples can be employed, although exemplary methods include, e.g., phlebotomy, swab (e.g., buccal swab), lavage, or fine needle aspirate biopsy procedure. Biological samples can also be obtained from bone marrow or spleen.

In some embodiments, a protein extract may be prepared from a biological sample. In some embodiments, a protein extract contains the total protein content. Methods of protein extraction are well known in the art. See, e.g., Roe (2001) “Protein Purification Techniques: A Practical Approach”, 2nd Edition, Oxford University Press. Numerous different and versatile kits can be used to extract proteins from bodily fluids and tissues, and are commercially-available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.).

Methods for obtaining and/or storing samples that preserve the activity or integrity of cells in the biological sample are well known to those skilled in the art. For example, a biological sample can be further contacted with one or more additional agents such as appropriate buffers and/or inhibitors, including protease inhibitors, the agents meant to preserve or minimize changes (e.g., changes in osmolarity or pH) in protein structure. Such inhibitors include, for example, chelators such as ethylenediamine tetraacetic acid (EDTA), ethylene glycol tetraacetic acid (EGTA), protease inhibitors such as phenylmethylsulfonyl fluoride (PMSF), aprotinin, and leupeptin. Appropriate buffers and conditions for storing or otherwise manipulating whole cells are described in, e.g., Pollard and Walker (1997), “Basic Cell Culture Protocols,” volume 75 of Methods in molecular biology, Humana Press; Masters (2000) “Animal cell culture: a practical approach,” volume 232 of Practical approach series, Oxford University Press; and Jones (1996) “Human cell culture protocols,” volume 2 of Methods in molecular medicine, Humana Press.

A sample also can be processed to eliminate or minimize the presence of interfering substances. For example, a biological sample can be fractionated or purified to remove one or more materials (e.g., cells) that are not of interest. Methods of fractionating or purifying a biological sample include, but are not limited to, flow cytometry, fluorescence activated cell sorting, and sedimentation.

### Therapeutic Methods

Also featured herein are therapeutic methods for treating subjects with a variety of conditions associated with fatty acid metabolism, including cancer, a metabolic syndrome, diabetes, obesity, atherosclerosis, or cardiovascular disease. For example, the disclosure features a method for treating a subject having a cancer comprising cancer cells with reduced PHD3 expression, methods for detection of which are described herein. The method comprises administering to the subject a compound that inhibits fatty acid metabolism, e.g., a fatty acid oxidation (FAO) inhibitor, in an amount effective to treat the cancer. In some embodiments, the cancer is one identified as having reduced PHD3 expression prior to administration of the FAO inhibitor. In some embodiments, the cancer is identified after treatment with the FAO inhibitor has been initiated and, in such embodiments, the methods can include reauthorizing or an affirmation of an order to administer the FAO inhibitor to the subject.

In some embodiments, the methods include receiving the results of a test determining that the subject's cancer comprises cancer cells with reduced PHD3 expression and, in view of this information, ordering administration of an effective amount of a compound that inhibits fatty acid metabolism, such as a fatty acid oxidation (FAO) inhibitor, to the subject. For example, a physician treating a subject can request that a third party (e.g., a CLIA-certified laboratory) to perform a test to determine whether a subject's cancer expresses PHD3 and the degree to which the cancer expresses PHD3. The laboratory may provide such information, or, in some embodiments, provide an expression score or value. If the cancer comprises cells with reduced expression of PHD3, the physician may then administer to the subject an inhibitor of fatty acid metabolism. Alternatively, the physician may order the administration of the inhibitor to the subject, which administration is performed by another medical professional, e.g., a nurse.

In some embodiments, the method can include: requesting a test, or the results of a test, which determines that the subject's cancer comprises cancer cells with reduced PHD3 expression; and administering or ordering administration of an effective amount of an inhibitor of fatty acid metabolism, such as a fatty acid oxidation (FAO) inhibitor, to the subject.

In some embodiments, the cancer is a prostate cancer. In some embodiments, the cancer is a glioblastoma or of hematological origin, e.g., an acute myeloid leukemia.

A “subject,” as used herein, can be any mammal. For example, a subject can be a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the subject is an infant (e.g., a human infant).

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an FAO inhibitor).

The term “preventing” is art-recognized, and when used in relation to a condition, is well understood in the art, and includes administration of a composition which reduces the frequency of, or delays the onset of, symptoms of a medical condition in a subject relative to a subject which does not receive the composition. For example, treatment with an PHD3 inhibitor may delay the onset of, and/or reduce the severity of symptoms upon onset of, a cardiovascular disorder.

In some embodiments, PHD3 expression by the cancer cells is less than or equal to 95 (e.g., less than or equal to 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1) % of normal cells of the same histological type from which the cancer cells are derived.

Inhibitors of fatty acid metabolism include, e.g., agents that inhibit fatty acid storage, agents that block fatty acid synthesis (e.g., ACC1 inhibitors), and inhibitors of FAO. In some embodiments, the FAO inhibitor is a carnitine palmitoyl transferase (CPT-I) inhibitor, such as etomoxir, oxfenicine, or perhexiline. In some embodiments, the CPT-I inhibitor is one identified in International Patent Application Publication Nos. WO 2009/156479, WO 2008/074692, WO 2008/015081, WO 2008/109991, and WO 2006/09220, and U.S. Pat. No. 5,196,418, the disclosures of each of which, as they relate to the compounds, are incorporated herein by reference in their entirety.

In some embodiments, the FAO inhibitor is a 3-ketoacyl-coenzyme A thiolase (3-KAT) inhibitor, such as trimetazidine or ranolazine. In some embodiments, the FAO inhibitor is a mitochondrial thiolase inhibitor, such as 4-bromocrotonic acid.

The disclosure also features a method for treating a subject having a cancer comprising cancer cells with elevated PHD3 expression, methods for detection of which are described above. The method comprises administering to the subject a compound that inhibits the glycolytic pathway, in an amount effective to treat the cancer. In some embodiments, the cancer is one identified as having elevated PHD3 expression prior to administration of the glycolytic pathway inhibitor. In some embodiments, the cancer is identified after treatment with the glycolytic pathway inhibitor has been initiated and, in such embodiments, the methods can include reauthorizing or an affirmation of an order to administer the glycolytic pathway inhibitor to the subject.

In some embodiments, the method include receiving the results of a test determining that the subject's cancer comprises cancer cells with elevated PHD3 expression and, in view of this information, ordering administration of an effective amount of a compound that inhibits the glycolytic pathway to the subject. For example, a physician treating a subject can request that a third party (e.g., a CLIA-certified laboratory) to perform a test to determine whether a subject's cancer expresses PHD3 and the degree to which the cancer expresses PHD3. The laboratory may provide such information, or, in some embodiments, provide an expression score or value. If the cancer comprises cells with elevated expression of PHD3, the physician may then administer to the subject an inhibitor of the glycolytic pathway. Alternatively, the physician may order the administration of the inhibitor to the subject, which administration is performed by another medical professional, e.g., a nurse.

In some embodiments, the method can include: requesting a test, or the results of a test, which determines that the subject's cancer comprises cancer cells with elevated PHD3 expression; and administering or ordering administration of an effective amount of an inhibitor of the glycolytic pathway to the subject.

In some embodiments, the cancer is a pancreatic cancer, kidney cancer, bladder cancer, melanoma, a lung cancer, a follicular lymphoma, a breast cancer, a colorectal cancer, or an ovarian cancer.

In some embodiments, the cancer cells express, or are determined to express, PHD3 mRNA or protein at a level at least 5 (e.g., at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160 170, 180, 190, 200, 300, 400, 500, or 1000)% higher than that of normal cells of the same histological type from which the cancer cells are derived. In some embodiments, the cancer cells express, or are determined to express, PHD3 mRNA or protein at a level at least 2 (e.g., 2.5, 3, 3.5, 4, 4.5, 5, 6, 7, 8, 9, 10, 20, 30, 40 50, 60 70, 80, 90, 100, 200, 300, 400, 500, 1000, 2000, 4000, 5000, or 10000) fold higher than that of normal cells of the same histological type from which the cancer cells are derived.

In some embodiments, the glycolytic pathway inhibitor is a hexokinase inhibitor, such as, but not limited to, 2-deoxyglucose, 3-bromopyruvate, or lonidamine. Suitable hexokinase inhibitors are known in the art and described in, e.g., U.S. Pat. Nos. 5,854,067; 8,119,116; 8,822,447; and International Patent Application Publication Nos. WO 2010/021750, WO 2011/127200, and WO 2012/018949.

In some embodiments, the glycolytic pathway inhibitor is a transketolase inhibitor, such as oxythiamine. Suitable transketolase inhibitors are known in the art and described in, e.g., International Patent Application Publication Nos. WO 2005/095344 and WO 2005/095391.

In some embodiments, the glycolytic pathway inhibitor is imatinib.

In some embodiments, the glycolytic pathway inhibitor is a glucose transporter (GLUT) inhibitor. Suitable GLUT inhibitors are known in the art and described in, e.g., International Patent Application Publication No. WO 2013/148994 and U.S. Patent Application Publication No. 20120252749.

In some embodiments, the glycolytic pathway inhibitor is a phosphofructokinase (PFK) inhibitor. In some embodiments, the glycolytic pathway inhibitor is a glyceraldehyde-3-phosphate dehydrogenase (GAPDH) inhibitor. In some embodiments, the glycolytic pathway inhibitor is a pyruvate kinase (PK) inhibitor. In some embodiments, the glycolytic pathway inhibitor is a lactate dehydrogenase (LDH) inhibitor. Suitable examples of each of the foregoing are known in the art.

The disclosure also features a method for treating a subject having a cancer comprising cancer cells with a reduced level of hydroxylation of ACC2 at proline 450 (or 343 or 2131) relative to SEQ ID NO:2, methods for detection of which are described above. The method comprises administering to the subject a compound that inhibits fatty acid metabolism, e.g., a fatty acid oxidation (FAO) inhibitor, in an amount effective to treat the cancer. In some embodiments, the cancer is one identified as having a reduced level of hydroxylation of ACC2 at proline 450 relative to SEQ ID NO:2 prior to administration of the FAO inhibitor. In some embodiments, the cancer is identified after treatment with the FAO inhibitor has been initiated and, in such embodiments, the methods can include reauthorizing or an affirmation of an order to administer the FAO inhibitor to the subject.

In some embodiments, the method include receiving the results of a test determining that the subject's cancer comprises cancer cells with a reduced level of hydroxylation of ACC2 at proline 450 (or 343 or 2131) relative to SEQ ID NO:2 and, in view of this information, ordering administration of an effective amount of a compound that inhibits fatty acid metabolism, such as a fatty acid oxidation (FAO) inhibitor, to the subject. For example, a physician treating a subject can request that a third party (e.g., a CLIA-certified laboratory) to perform a test to determine the degree to which ACC2 is hydroxylated at proline 450 relative to SEQ ID NO:2 by the subject's cancer cells. The laboratory may provide such information, or, in some embodiments, provide an expression score or value. If the cancer comprises cells with a reduced level of hydroxylation of ACC2 at proline 450 (or 343 or 2131) relative to SEQ ID NO:2, the physician may then administer to the subject an inhibitor of fatty acid metabolism. Alternatively, the physician may order the administration of the inhibitor to the subject, which administration is performed by another medical professional, e.g., a nurse.

In some embodiments, the method can include: requesting a test, or the results of a test, which determines that the subject's cancer comprises cancer cells with a reduced level of hydroxylation of ACC2 (e.g., at proline 450 relative to SEQ ID NO:2); and administering or ordering administration of an effective amount of an inhibitor of fatty acid metabolism, such as a fatty acid oxidation (FAO) inhibitor, to the subject.

In some embodiments, the cancer is a prostate cancer. In some embodiments, the cancer is a glioblastoma or of hematological origin, e.g., an acute myeloid leukemia.

In some embodiments, level of hydroxylation of ACC2 (e.g., at proline 450 relative to SEQ ID NO:2) in the cancer cells is less than or equal to 95 (e.g., less than or equal to 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1) % of normal cells of the same histological type from which the cancer cells are derived.

The disclosure also features a method for treating a subject having a cancer comprising cancer cells with an elevated level of hydroxylation of ACC2 (e.g., at proline 450 relative) to SEQ ID NO:2, methods for detection of which are described above. The method comprises administering to the subject a compound that inhibits the glycolytic pathway, in an amount effective to treat the cancer. In some embodiments, the cancer is one identified as having an elevated level of hydroxylation of ACC2 at proline 450 relative to SEQ ID NO:2 prior to administration of the glycolytic pathway inhibitor. In some embodiments, the cancer is identified after treatment with the glycolytic pathway inhibitor has been initiated and, in such embodiments, the methods can include reauthorizing or an affirmation of an order to administer the glycolytic pathway inhibitor to the subject.

In some embodiments, the method include receiving the results of a test determining that the subject's cancer comprises cancer cells with an elevated level of hydroxylation of ACC2 at proline 450 relative to SEQ ID NO:2, and, in view of this information, ordering administration of an effective amount of a compound that inhibits the glycolytic pathway to the subject. For example, a physician treating a subject can request that a third party (e.g., a CLIA-certified laboratory) to perform a test to determine the degree to which ACC2 is hydroxylated at proline 450 relative to SEQ ID NO:2 in the cancer cells. The laboratory may provide such information, or, in some embodiments, provide an expression score or value. If the cancer comprises cells with an elevated level of hydroxylation of ACC2 at proline 450 relative to SEQ ID NO:2, the physician may then administer to the subject an inhibitor of the glycolytic pathway. Alternatively, the physician may order the administration of the inhibitor to the subject, which administration is performed by another medical professional, e.g., a nurse.

In some embodiments, the method can include: requesting a test, or the results of a test, which determines that the subject's cancer comprises cancer cells with an elevated level of hydroxylation of ACC2 (e.g., at proline 450 relative to SEQ ID NO:2); and administering or ordering administration of an effective amount of an inhibitor of the glycolytic pathway to the subject.

In some embodiments, the cancer is a pancreatic cancer, kidney cancer, bladder cancer, melanoma, a lung cancer, a follicular lymphoma, a breast cancer, a colorectal cancer, or an ovarian cancer.

In some embodiments, the level of hydroxylation of ACC2 (e.g., at proline 450 relative to SEQ ID NO:2) by the cancer cells is at least 5 (e.g., at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160 170, 180, 190, 200, 300, 400, 500, or 1000) % higher than that of normal cells of the same histological type from which the cancer cells are derived. In some embodiments, the level of hydroxylation of ACC2 at proline 450 relative to SEQ ID NO:2 by the cancer cells is at least 2 (e.g., 2.5, 3, 3.5, 4, 4.5, 5, 6, 7, 8, 9, 10, 20, 30, 40 50, 60 70, 80, 90, 100, 200, 300, 400, 500, 1000, 2000, 4000, 5000, or 10000) fold higher than that of normal cells of the same histological type from which the cancer cells are derived.

Also featured are methods for sensitizing cancer cells to inhibitors of fatty acid metabolism, which are useful for, inter alia, treating cancer. The methods include administering to the subject an inhibitor of PHD3 to thereby sensitize the cancer to an inhibitor of fatty acid metabolism, such as a fatty acid oxidation (FAO) inhibitor; and administering to the subject an effective amount of a FAO inhibitor to treat the cancer, wherein the effective amount of the inhibitor is lower than the amount effective to treat the cancer in the absence of PHD3 inhibition. Suitable classes of PHD3 inhibitors are discussed herein. In some embodiments, the PHD3 inhibitor is a small molecule, such as, but not limited to, those described in International Patent Application Publication Nos. WO 2008/135639, WO 2013063221, and WO 2013/032893, and U.S. Patent Application Publication No. US 20140256722. In some embodiments, the PHD3 inhibitor is an antisense oligonucleotide, e.g., an siRNA or shRNA.

In some embodiments, the amount of the fatty acid metabolism inhibitor to be effective in a subject sensitized with the PHD3 inhibitor is less than or equal to 95 (e.g., less than or equal to 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1) % of the amount required for the same level of efficacy in the absence of sensitization.

The inhibitor compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

As used herein the term “effective amount” or “therapeutically effective amount”, in an in vivo setting, means a dosage sufficient to treat, inhibit, or alleviate one or more symptoms of the disorder being treated or to otherwise provide a desired pharmacologic and/or physiologic effect (e.g., modulate (e.g., enhance) an immune response to an antigen. The precise dosage will vary according to a variety of factors such as subject-dependent variables (e.g., age, immune system health, etc.), the disease, and the treatment being effected.

Suitable human doses of any of the compounds described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such compositions can be determined by known pharmaceutical procedures in cell cultures or experimental animals (e.g., animal models of cancer, cardiovascular disease, or metabolic disorders). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such compounds lies generally within a range of circulating concentrations of the compounds that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal modeling can be used to determine a dose required to achieve a therapeutically effective concentration within the local site.

In some embodiments of any of the methods described herein, an agent can be administered to a mammal in conjunction with one or more additional therapeutic agents.

Suitable additional anti-cancer therapies include, e.g., chemotherapeutic agents, ionizing radiation, immunotherapy agents, or hyperthermotherapy. Chemotherapeutic agents include, but are not limited to, aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, taxol, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF)-inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

The term “immunotherapeutic agent” can include any molecule, peptide, antibody or other agent which can stimulate a host immune system to generate an immune response to a tumor or cancer in the subject. Various immunotherapeutic agents are useful in the compositions are known in the art and include, e.g., PD-1 and/or PD-1L inhibitors, CD200 inhibitors, CTLA4 inhibitors, and the like. Exemplary PD-1/PD-L1 inhibitors (e.g., anti-PD-1 and/or anti-PD-L1 antibodies) are known in the art and described in, e.g., International Patent Application Publication Nos. WO 2010036959 and WO 2013/079174, as well as U.S. Pat. Nos. 8,552,154 and 7,521,051, the disclosures of each of which as they relate to the antibody descriptions are incorporated herein by reference in their entirety. Exemplary CD200 inhibitors are also known in the art and described in, e.g., International Patent Application Publication No. WO 2007084321. Suitable anti-CTLA4 antagonist agents are described in International Patent Application Publication Nos. WO 2001/014424 and WO 2004/035607; U.S. Patent Application Publication No. 2005/0201994; and European Patent No. EP 1212422. Additional CTLA-4 antibodies are described in U.S. Pat. Nos. 5,811,097, 5,855,887, 6,051,227, and 6,984,720. It is understood that the immunomodulatory agents can also be used in conjunction with a compound described herein for the treatment of an infection, such a viral, bacterial, or fungal infection, or any other condition in which an enhanced immune response to an antigen of interest would be therapeutically beneficial.

The disclosure also features a method for increasing fatty acid oxidation by a cell, which includes contacting the cell with a compound that inhibits the hydroxylation of ACC2 at proline 450 relative to SEQ ID NO:2 by PHD3 in an amount effective to increase fatty acid oxidation by the cell. The methods can be cell-based or in vivo.

For example, the disclosure features a method for increasing fatty acid oxidation in a subject in need thereof. The method comprises administering to the subject a compound that inhibits the hydroxylation of ACC2 at proline 450 relative to SEQ ID NO:2 by PHD3 in an amount effective to increase fatty acid oxidation in the subject. Also featured are methods for promoting weight loss in a subject, which methods comprise administering to the subject a compound that inhibits the hydroxylation of ACC2 at proline 450 relative to SEQ ID NO:2 by PHD3 in an amount effective to promote weight loss in the subject.

The disclosure also features a method for treating cardiovascular disease in a subject, the method comprising administering to the subject a compound that inhibits the hydroxylation of ACC2 at proline 450 relative to SEQ ID NO:2 by PHD3 in an amount effective to treat the cardiovascular disease in the subject. Also featured is a method for treating a subject afflicted with a metabolic syndrome, diabetes, obesity, atherosclerosis, or cardiovascular disease, the method comprising administering to the subject a compound that inhibits the hydroxylation of ACC2 at proline 450 relative to SEQ ID NO:2 by PHD3 in an amount effective to treat the metabolic syndrome, diabetes, obesity, atherosclerosis, or cardiovascular disease. In some embodiments, the disclosure features a method for delaying on the onset of, and/or reducing the severity of symptoms at onset of, a metabolic syndrome, diabetes, obesity, atherosclerosis, or cardiovascular disease. The method includes administering to the subject a compound that inhibits the hydroxylation of ACC2 at proline 450 relative to SEQ ID NO:2 by PHD3 in an amount effective to delaying on the onset of, and/or reducing the severity of symptoms at onset of, a metabolic syndrome, diabetes, obesity, atherosclerosis, or cardiovascular disease.

In some embodiments, the subject has cardiovascular disease. Cardiovascular disease (CVD) is the general term for heart and blood vessel diseases, including atherosclerosis, coronary heart disease, cerebrovascular disease, aorto-iliac disease, and peripheral vascular disease. Subjects with CVD may develop a number of complications, including, but not limited to, myocardial infarction, stroke, angina pectoris, transient ischemic attacks, congestive heart failure, aortic aneurysm and death. CVD accounts for one in every two deaths in the United States and is the number one killer disease. Thus, prevention of cardiovascular disease is an area of major public health importance.

In some embodiments, the subject has a metabolic disorder. As used herein, a metabolic disorder can be any disorder associated with metabolism, and examples include but are not limited to, obesity, central obesity, insulin resistance, glucose intolerance, abnormal glycogen metabolism, type 2 diabetes, hyperlipidemia, hypoalbuminemia, hypertriglyceridemia, metabolic syndrome, syndrome X, a fatty liver, fatty liver disease, polycystic ovarian syndrome, and acanthosis nigricans. In one embodiment, the methods are directed towards treating at least one component of postprandial metabolism, such as, but not limited to hepatic glycogen synthesis, protein synthesis and clearance of plasma glucose.

In some embodiments, the subject is overweight or obese. “Obesity” refers to a condition in which the body weight of a mammal exceeds medically recommended limits by at least about 20%, based upon age and skeletal size. “Obesity” is characterized by fat cell hypertrophy and hyperplasia. “Obesity” may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased Fat Mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65(2): 432-8 and Butte et al. (1999) Am J Clin Nutr 69(2): 299-307. “Overweight” individuals are generally having a body mass index (BMI) between 25 and 30. “Obese” individuals or individuals suffering from “obesity” are generally individuals having a BMI of 30 or greater. Obesity may or may not be associated with insulin resistance.

In some embodiments, the subject has an obesity-related disorder. An “obesity-related disease” or “obesity related disorder” or “obesity related condition”, which are all used interchangeably, refers to a disease, disorder, or condition, which is associated with, related to, and/or directly or indirectly caused by obesity. The “obesity-related diseases”, or the “obesity-related disorders” or the “obesity related conditions” include but are not limited to, coronary artery disease/cardiovascular disease, hypertension, cerebrovascular disease, stroke, peripheral vascular disease, insulin resistance, glucose intolerance, diabetes mellitus, hyperglycemia, hyperlipidemia, dyslipidemia, hypercholesteremia, hypertriglyceridemia, hyperinsulinemia, atherosclerosis, cellular proliferation and endothelial dysfunction, diabetic dyslipidemia, HIV-related lipodystrophy, peripheral vessel disease, cholesterol gallstones, cancer, menstrual abnormalities, infertility, polycystic ovaries, osteoarthritis, sleep apnea, metabolic syndrome (Syndrome X), type II diabetes, diabetic complications including diabetic neuropathy, nephropathy, retinopathy, cataracts, heart failure, inflammation, thrombosis, congestive heart failure, and any other cardiovascular disease related to obesity or an overweight condition and/or obesity related asthma, airway and pulmonary disorders.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a particular condition. An individual having one or more of these risk factors has a higher probability of developing a condition than an individual without these risk factors. Examples (i.e., categories) of risk groups are well known in the art and discussed herein, such as smoking (risk of cancer) and high-fat diets or elevated LDL levels (obesity and/or heart disease).

In some embodiments, the inhibitor of PHD3 is administered in conjunction with one or more additional agents useful for treating a metabolic syndrome, diabetes, obesity, atherosclerosis, or cardiovascular disease. For example, for cardiovascular disorders, the PHD3 inhibitors can be administered in conjunction with an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 inhibitor, an angiotensin system inhibitor, and/or combinations thereof. The agent is administered in an amount effective to lower the risk of the subject developing a future cardiovascular disorder.

“Anti-inflammatory” agents include but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

“Anti-thrombotic” and/or “fibrinolytic” agents include but are not limited to, Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

“Anti-platelet” agents include but are not limited to, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide.

“Lipid-reducing” agents include but are not limited to, gemfibrozil, cholystyramine, colestipol, nicotinic acid, probucol lovastatin, fluvastatin, simvastatin, atorvastatin, pravastatin, cerivastatin, and other HMG-CoA reductase inhibitors.

“Direct thrombin inhibitors” include but are not limited to, hirudin, hirugen, hirulog, agatroban, PPACK, thrombin aptamers.

“Glycoprotein IIb/IIIa receptor inhibitors” are both antibodies and non-antibodies, and include but are not limited to ReoPro (abcixamab), lamifiban, tirofiban.

“Calcium channel blockers” are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogenous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, aminone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

“Beta-adrenergic receptor blocking agents” are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hyd-roxypropoxy)-3-pyridenecarbonitrilHCl, 1-butylamino-3-(2,5-dichlorophenoxy-)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

Suitable COX-2 inhibitors include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors”; U.S. Pat. No. 5,677,318 Diphenyl-1, 2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-d-ihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

An “angiotensin system inhibitor” is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin 1 from which angiotensin II is ultimately derived. The renin-angiotensin system is involved in the regulation of hemodynamics and water and electrolyte balance.

Angiotensin (renin-angiotensin) system inhibitors are compounds that act to interfere with the production of angiotensin II from angiotensinogen or angiotensin I or interfere with the activity of angiotensin II. Such inhibitors are well known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists.

Examples of angiotensin II antagonists include: peptidic compounds (e.g., saralasin, [(San1)(Val5)(Ala8)]angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4, 5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF 108566 (E-alpha-2-[2-butyl-1-(carboxy phenyl)methyl]1H-imidazole-5-yl[methylane]-2-thiophenepropanoic acid, Smith Kline Beecham Pharmaceuticals, Pa.); Losartan (DUP7531MK954, DuPont Merck Pharmaceutical Company); Remikirin (CR042-5892, F. Hoffman LaRoche AG); A.sub.2 agonists (Marion Merrill Dow) and certain non-peptide heterocycles (G. D. Searle and Company). Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamrates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

The following examples are meant to illustrate, not to limit, the disclosure.

### EXAMPLES

### Example 1. PHD3 Interacts with ACC2 and Modulates FAO

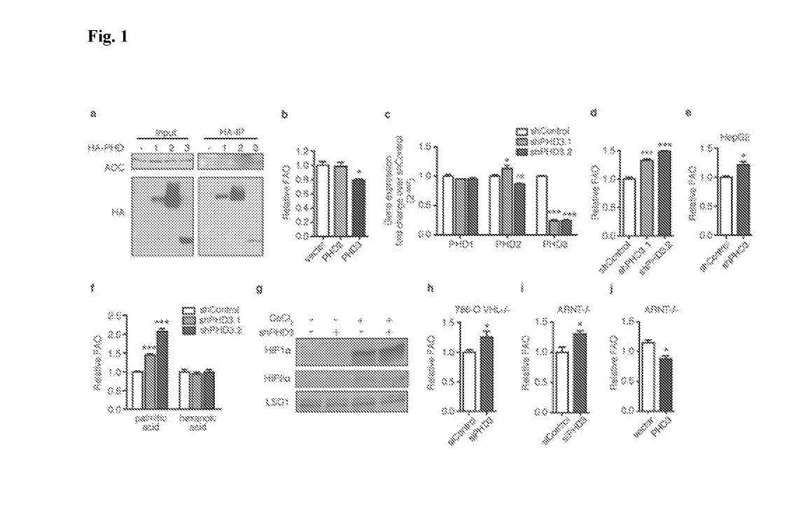

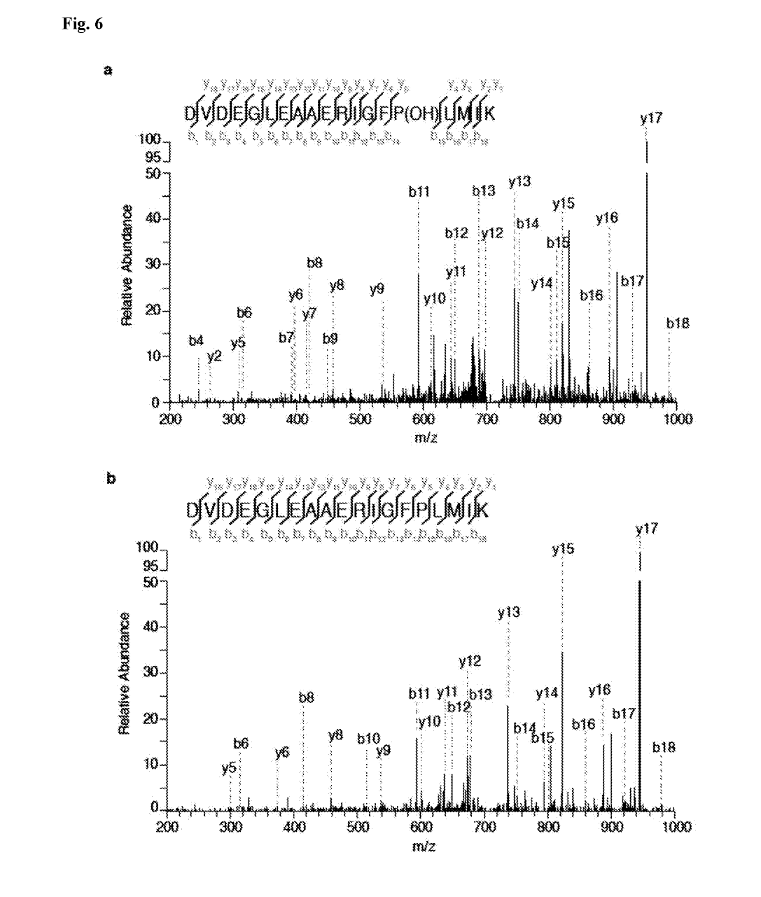

In order to identify novel PHD3 substrates, immunoprecipitation of PHD3 were performed followed by liquid chromatography tandem mass spectrometry (LC-MS2). A novel interaction between PHD3 and acetyl-CoA carboxylase (ACC) was detected. ACC specifically interacted with PHD3 but not PHD1, PHD2, or anti-HA affinity resin alone, as verified by Western blot (FIG. 1, Panel A). ACC is a pivotal regulator of fat metabolism that directs the cell to catabolize or synthesize fatty acids by converting acetyl-coA to malonyl-CoA, which serves as a precursor for fat synthesis and an inhibitor of fatty acid oxidation (FAO) (References 19, 20, and 21). To test if PHD3 impacts fatty acid utilization, the oxidation of palmitate was measured in cells in which PHD3 was overexpressed or missing (by way of siRNA-mediated knockdown). Overexpression of PHD3, but not PHD2, inhibited palmitate oxidation (FIG. 1, Panel B), and conversely knockdown of PHD3 enhanced palmitate oxidation in 293T cells (FIG. 1, Panels C and D, and FIG. 6, Panel A). This showed that PHD3 has an inhibitory effect on FAO, a finding confirmed in HepG2 cells (FIG. 1, Panel E, and FIG. 6, Panel B). PHD1 and PHD2 gene expression were not consistently altered by PHD3 knockdown, indicating the effect on FAO was not due to over-compensation by other PHDs (FIG. 1, Panel C). PHD3 modulates FAO at a magnitude similar to that observed in studies of known lipid metabolism regulators including ACC, adiponectin and sirtuins (References 22-25).

Since ACC gates long chain fatty acid import into the mitochondria, whereas short chain fatty acids can freely diffuse, a series of experiments were conducted to determine whether PHD3 specifically modulates oxidation of long chain fatty acids. Comparison of 16-carbon palmitate oxidation versus 6-carbon hexanoate oxidation revealed PHD3 knockdown only boosts long chain FAO (FIG. 1, Panel F). This indicates that PHD3 knockdown represses ACC, allowing increased flux of long chain fatty acids into the mitochondria for utilization as fuel.

### Example 2. PHD3 Modulation of FAO is Independent of HIF

Next a multifaceted approach was used to systematically assess whether elevated FAO caused by PHD3 knockdown was due to HIF stabilization. HIF1/2α protein levels were not changed with PHD3 knockdown under the experimental conditions (FIG. 1, Panel G), indicating that the effects of PHD3 on FAO are not due to altered HIF. Furthermore, PHD3 modulated FAO in cellular systems where HIF is either constitutively stabilized or inactivated. PHD3 knockdown increased FAO in 786-O von Hippel-Lindau (VHL)-deficient renal carcinoma cells and hypoxia-treated 293T cells, each are cellular conditions in which HIF is stabilized (FIG. 1, Panel H, and FIG. 6, Panels C and D). Additionally, PHD3 alters FAO in mouse hepatoma 4 (B13NBii1) arylhydrocarbon receptor nuclear translocator (ARNT, also known as HIFβ) null cells, which lack functional HIF transcriptional activity (FIG. 1, Panels I-J and FIG. 6, Panel E). Together, these multiple lines of data indicate PHD3 inhibits FAO independently of HIF.

Also assessed whether PHD3 activity toward ACC2 and FAO is sensitive to the cellular nutrient status. In ARNT−/− hepatoma cells expressing endogenous levels of PHD3, FAO was observed to be limited to a basal level under high nutrient conditions with complete media, but reaches higher levels under low nutrient conditions, consisting of serum-free, low glucose media. However, PHD3 overexpression blunts the increase in FAO that otherwise occurs in a low nutrient state. This raises the possibility that PHD3 is sensitive to nutrient availability and bioenergetic status and consequently adjusts fatty acid utilization. These data fit the hypothesis that greater activity of endogenous PHD3 in the presence of abundant nutrients restricts FAO, while reduced PHD3 activity upon nutrient deprivation causes repression of FAO to be lifted.

Next ACC hydroxylation under high and low nutrient conditions was examined. ACC is strongly hydroxylated by endogenous PHD3 in 293T cells grown in complete media, but less hydroxylated in cells grown in serum-free, low glucose media, suggesting PHD3 is active under nutrient replete conditions. In low nutrient conditions, overexpressing PHD3 restores the level of hydroxylation to nearly that of cells in the high nutrient state. Thus, these data suggest endogenous PHD3 hydroxylates ACC2 under nutrient replete conditions to limit FAO, but is less active under nutrient deprivation. This model is further supported by the observation that PHD3 expression is higher in 293T cells grown in complete media compared to low nutrient media.

### Example 3. PHD3 Hydroxylates ACC2 at Proline 450

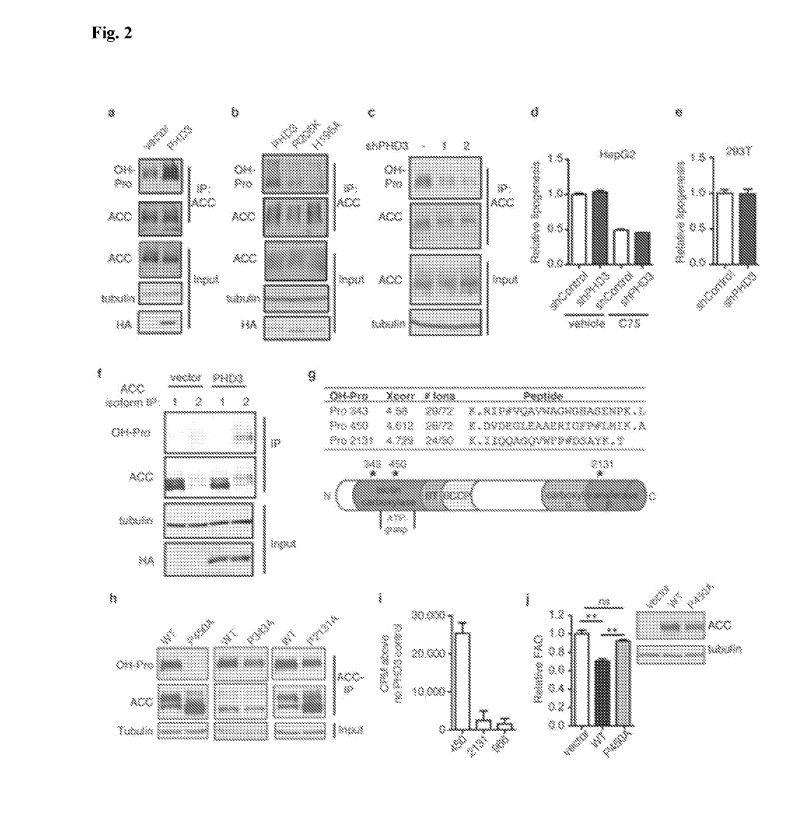

To test the ability of PHD3 to directly modify ACC, PHD3-responsive changes in prolyl hydroxylation were monitored. ACC was found to be hydroxylated, and hydroxylation is increased with PHD3 overexpression (FIG. 2, Panel A). Two previously characterized catalytically inactive PHD3 mutants, H196A and R206K (References 25 and 26), did not augment ACC hydroxylation to the same extent as wild type PHD3 (FIG. 2, Panel B). Furthermore, knockdown of PHD3 decreased ACC hydroxylation (FIG. 2, Panel C). ACC is present in two spatially and functionally distinct isoforms. Cytosolic ACC1 provides malonyl-CoA for fatty acid synthesis, while ACC2 at the outer mitochondrial membrane generates malonyl-CoA to inhibit the fatty acid transport protein CPT1 (Reference 19). Several PHD3-interacting peptides found by mass spectrometry are shared between ACC1 and ACC2 (Table 1).

TABLE 1
PHD3-interacting peptides indistinguishable
between ACC1 and 2 isozymes (SEQ ID NOS 14-18,
respectively, in order of appearance).

| | | # | Redun- | Peptide |
|---|---|---|---|---|
| Xcorr | ΔCorr | Ions | dancy | |
| 4.518 | 0.54 | 33/88 | 8 | R.ITSENPDEGFKPSSGTVQELNFR.S |
| 3.168 | 0.392 | 16/24 | 12 | R.DFTVASPAEFVTR.F |
| 2.409 | 0.071 | 15/24 | 9 | K.EASFEYLQNEGER.L |
| 2.221 | 0.415 | 16/18 | 9 | R.AIGIGAYLVR.L |
| 1.757 | 0.124 | 8/14 | 8 | K.DMYDQVLK.F |

Peptides were filtered using Xcorr and ΔCorr. Xcorr = cross correlation score. ΔCorr = delta correlation.

Next, a series of experiments were performed to determine if PHD3 hydroxylates ACC1 or ACC2. PHD3 regulates FAO, but no effects on fatty acid synthesis on cell lines tested were observed (FIG. 2, Panels D and E), indicating that PHD3 may specifically hydroxylate and regulate ACC2. Immunoprecipitation of endogenous ACC1 or ACC2 by isoform-specific antibodies showed hydroxylation was particular to ACC2 and also stronger in the presence versus absence of PHD3 (FIG. 2, Panel F), demonstrating PHD3 is a direct modulator of ACC2 hydroxylation status.

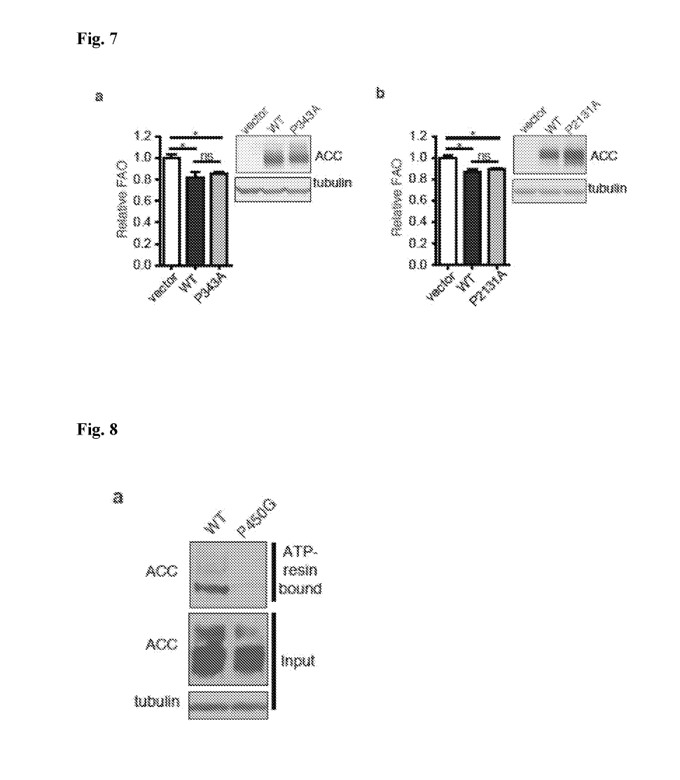

Liquid chromatography coupled to tandem mass spectrometry (LC-MS²) was used to map ACC2 proline residues that were modified by hydroxylation, and three hydroxylated prolines with greater than 5 redundant peptides per hydroxylation site: prolines 343, 450 and 2131 were discovered. These sites are located in the biotin carboxylase, ATP-grasp and carboxyltransferase domains, respectively (FIG. 2, Panel G, representative spectra in FIG. 7, Panels A and B). To further examine hydroxylation at these residues, proline to alanine ACC2 point mutants were generated at each putative hydroxylation site. Immunoprecipitation of wild type or mutant ACC2 revealed P450A mutagenesis most dramatically decreased the level of hydroxylation compared to P343A and P2131A variants (FIG. 2, Panel H). Using a reconstituted in vitro radioactivity-based hydroxylation assay, recombinant PHD3 was shown to hydroxylate a synthetic ACC2 peptide containing P450, but not a peptide containing P2131 or a control ACC2 proline-containing peptide (P966) (FIG. 2, Panel I). To test whether residue P450 impacts ACC2 biology, FAO experiments were performed in 293T cells overexpressing wild-type or proline to alanine variants of ACC2. While overexpression of wild type ACC2, or the P343A and P2131A variants all decreased FAO, the P450A mutant lacking the major hydroxylation site had blunted ability to repress FAO (FIG. 2, Panel J and FIG. 8, Panels A and B). Together, these data demonstrate P450 is a major site of PHD3 hydroxylation and a key regulator of ACC2 function.

### Example 4. Hydroxylation of ACC2 Modulates ACC2 Enzymatic Activity

At only 16 daltons, prolyl hydroxylation is among the smallest of all posttranslational modifications. Nevertheless, the electronegativity it imparts can induce conformational changes in the prolyl peptide bond significant enough to alter protein-protein interactions, substrate stability or activity (References 3 and 28). Thus, the role of site-specific hydroxylation in ACC2 activity was investigated. Residue P450 is conserved from yeast to human (FIG. 3, Panel A) and is located in the ATP-grasp domain, a 196 amino acid region within the biotin carboxylase domain that includes nucleotide-binding amino acids at residues 458-463 (Reference 29). To evaluate the link between PHD3 and ACC enzymatic activity, in vitro ACC activity assays were performed based on the production of [¹⁴C]malonyl-CoA from [¹⁴C]bicarbonate and acetyl-CoA. Although endogenous ACC activity was barely detectable in whole cell lysates, overexpression of ACC2 enabled detection. ACC2 was activated by citrate, a known allosteric modulator (Reference 30), while P450A mutation strongly decreased ACC activity (FIG. 3, Panel B). When assaying the effect of PHD3 on ACC2 function, PHD3 overexpression amplified wild-type ACC2 activity (FIG. 3, Panel C), but had no effect on the P450A variant. These data collectively support the model that PHD3 activates ACC2 via hydroxylation of P450 in order to repress FAO (FIG. 3, Panel D).

To gain mechanistic insight into how hydroxylation activates ACC2, P450 was site mapped in the published human ACC2 biotin carboxylase domain crystal structure (PDB: 3JRW) (Reference 31) (FIG. 3, Panel E). Superposition of this model with the E. coli ATP-bound ACC biotin carboxylase domain (PDB: 1DV2) (Reference 32) showed that P450 is in close proximity to the catalytic site ATP. P450 caps the adenine ring of ATP, while the phosphate groups of ATP abut the previously described nucleotide-binding site within ACC2. The proximity of P450 and ATP indicated that PHD3 may promote ATP-binding by ACC2, which was assessed by immunoprecipitation with ATP-agarose. With knockdown of PHD3, ATP-binding by endogenous ACC2 was diminished (FIG. 3, Panel F). Further, ACC2 proteins lacking the major hydroxylation site due to P450 mutation to either alanine or glycine showed decreased ATP-binding versus wild type ACC2 (FIG. 3, Panel G and FIG. 8, Panel A). Together these data indicate PHD3 activates ACC2 by enabling greater affinity for the co-substrate ATP.

### Example 5. PHD3 Expression and Cancer

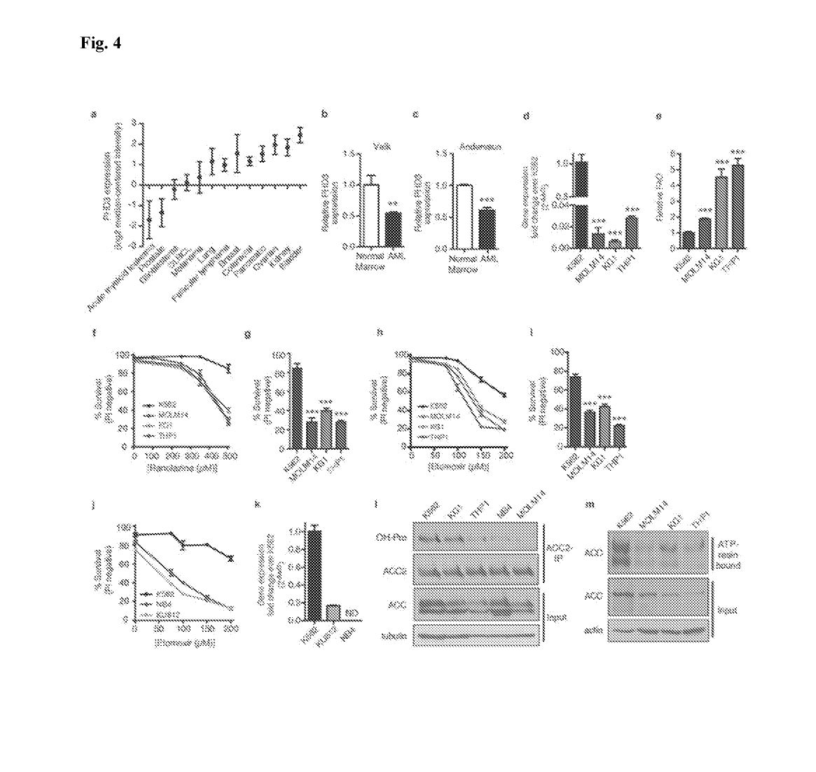

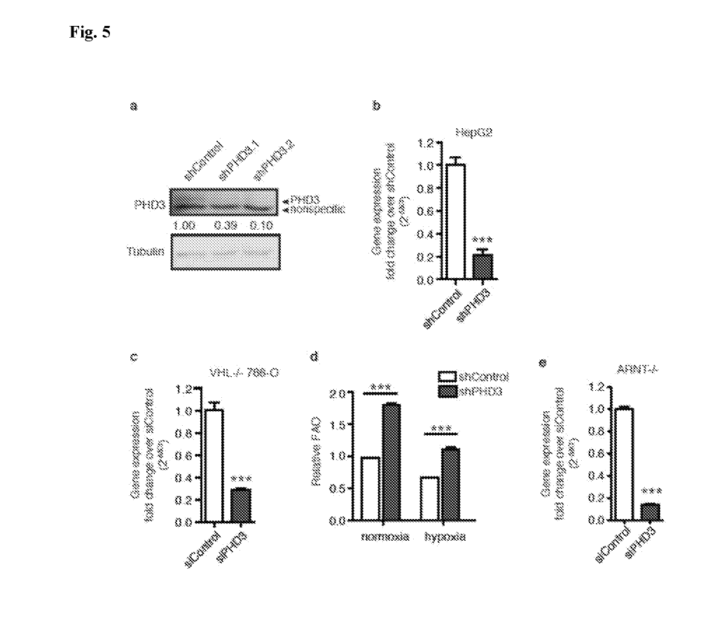

To test a hypothesis that loss of PHD3 provides a mechanism for increased FAO dependency in cancer, first the Ramaswamy Multi-Cancer dataset (Reference 35) from the Oncomine cancer microarray database (http://www.oncomine.org) was analyzed, and it indicated that AML has the lowest PHD3 expression compared to a panel of other cancerous tissues (FIG. 4, Panel A). Valk Leukemia (285 AML and 8 normal marrow samples) and Andersson Leukemia (23 AML and 6 normal marrow samples) datasets also show decreased PHD3 mRNA levels in AML compared to normal marrow patient samples (FIG. 4, Panels B and C) (References 36 and 37).

To define further a role for PHD3 in leukemia, the metabolic consequences of low PHD3 expression in a panel of leukemia cell lines were examined. Gene expression studies revealed that PHD3 is markedly decreased in a panel of AML cell lines (MOLM14, KG1, THP1) compared to the K562 chronic myeloid leukemia (CML) cell line (FIG. 4, Panel D). Low PHD3 expression in AML cells correlated with 2 to 5 times greater palmitate oxidation (FIG. 4, Panel E). It was hypothesized that low-PHD3 leukemia cells possessed a metabolic liability rooted by their dependency on FAO. Thus, a series of experiments were performed to evaluate low-PHD3 leukemia cells to sensitivity to ranolazine or etomoxir, FAO inhibitors that have shown success in treating angina and heart disease, respectively (References 5, 38, and 39). Ranolazine inhibits 3-ketoacylthiolase, the enzyme catalyzing the final step in each round of β-oxidation, and etomoxir represses FAO by inhibiting CPT1 (Reference 5). 96 hour inhibition of FAO by ranolazine drastically reduced cell viability in low-PHD3 leukemia cells while viability was largely maintained for K562 leukemia cells with higher PHD3 (FIG. 4, Panels F and H). Additionally, 96 hour treatment with etomoxir led to substantial cell death in low-PHD3 leukemia cells, but not K562. (FIG. 4, Panels G and I). Sensitivity to FAO inhibition was more strongly linked to PHD3 status than to classification as AML or CML. A CML cell line with low PHD3 expression, KU812, was in fact sensitive to treatment with etomoxir and more closely mimicked another low-PHD3 AML cell line (NB4) rather than a high-PHD3 CML line (K562) (FIG. 4, Panels J and K). Finally leukemia cell lines with decreased PHD3 levels also showed less ACC hydroxylation and ATP binding (FIG. 4, Panels L and M). Thus, these data indicate PHD3 gene expression corresponds to the vulnerability of cancer cells to pharmacological inhibition of FAO and could be considered as a marker to predict the metabolic demands of a particular cancer.

### Example 6—PHD3 Regulates FAO Through a Mechanism Independent of AMPK

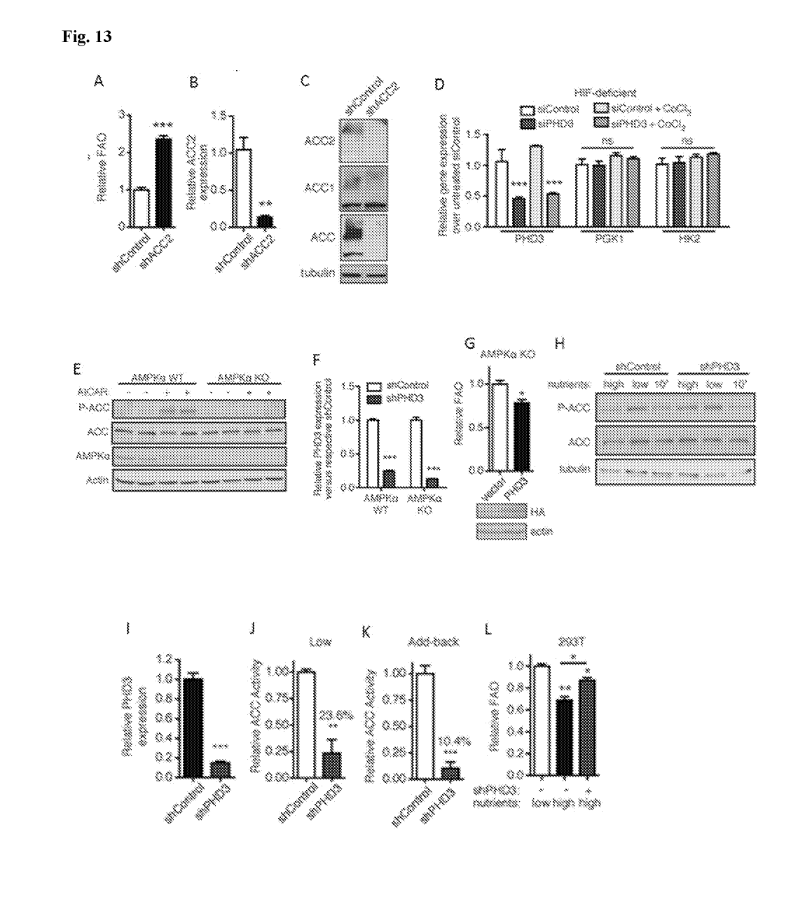

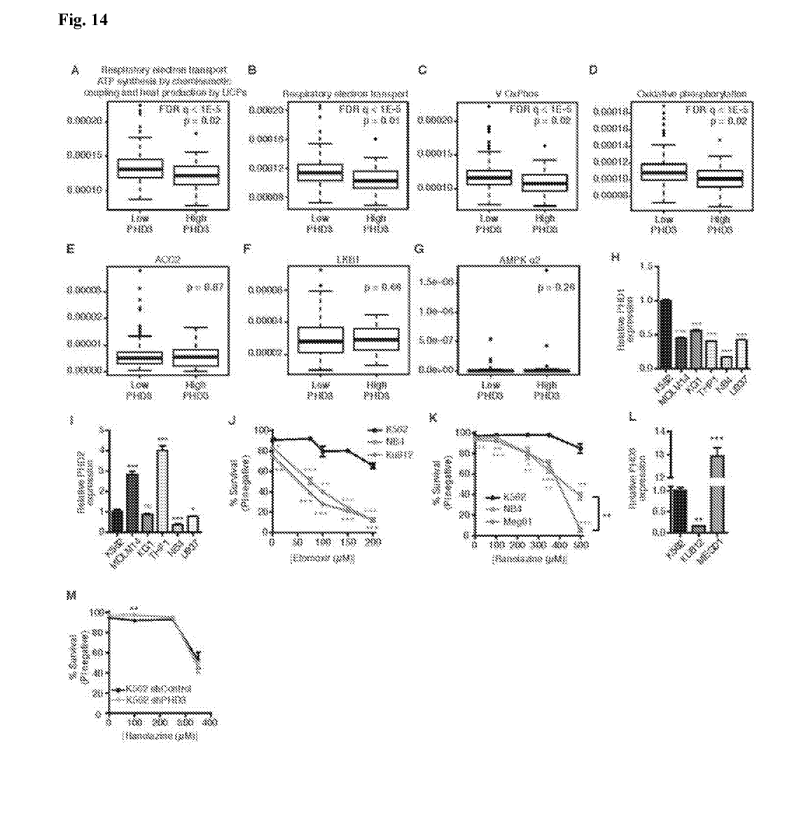

Whether PHD3 activates ACC2 in concert with the major known regulator of this metabolic node, AMP-activated protein kinase (AMPK) was examined. Upon detecting a low cellular energy status, AMPK inhibits ACC2 by phosphorylating serine 222, disrupting the dimer-dimer interface to block formation of the more active ACC oligomer. In this way, AMPK activates FAO as part of a general program to restore cellular ATP levels. To test the interdependency of PHD3 and AMPK, the ability of PHD3 to modulate fatty acid catabolism in systems lacking AMPK activity was assessed. PHD3 knockdown amplified FAO in both wildtype and AMPKα-knockout mouse embryonic fibroblasts (MEFs) (FIG. 10, Panel A; validation of AMPKα-knockout and extent of PHD3 knockdown are shown in FIG. 13, Panel E, and FIG. 13, Panel F). Furthermore, PHD3 overexpression repressed FAO even in the absence of AMPK (FIG. 13, Panel G). Additionally, it was found that AMPK could phosphorylate ACC under low nutrient conditions in both control and PHD3-knockdown MEFs, and ACC phosphorylation was decreased upon return to abundant nutrients regardless of PHD3 status (FIG. 13, Panel H; extent of knockdown shown in FIG. 13, Panel I).

### Example 7—PHD3 Hydroxylates ACC and Represses FAO in Response to Nutrient Abundance

Maintaining energy homeostasis is critical to cellular function. Fatty acids are not a predominant fuel choice under nutrient replete conditions but rather are reserved for times of fasting or nutrient deprivation to restore metabolic homeostasis. During conditions of stress or low energy, cells ramp up ATP production by activating fatty acid oxidation via AMPK signaling. While AMPK boosts FAO by inhibiting ACC2, the data presented herein show PHD3 has the opposite effect of repressing FAO by activating ACC2. Thus, it was determined whether PHD3 might be a candidate for dynamically repressing FAO in response to nutrient abundance. To this end, it was found that in control vector-treated cells, endogenous ACC was strongly hydroxylated in cells grown in high glucose medium containing serum (high) versus cells treated 12 h with serum-free, low glucose medium (low) (FIG. 10, Panel B). Similarly, PHD3 overexpression in cells grown in low nutrient conditions restored ACC hydroxylation nearly to levels observed in the high nutrient state (FIG. 10, Panel B). This suggests that endogenous PHD3 hydroxylates and activates ACC particularly when nutrients are abundant. Further, PHD3 knockdown strongly decreased ACC hydroxylation in high nutrient medium (FIG. 10, Panel C left). In comparison, the effect of PHD3 knockdown on ACC hydroxylation is less evident in low nutrient conditions (FIG. 10, Panel C, right).

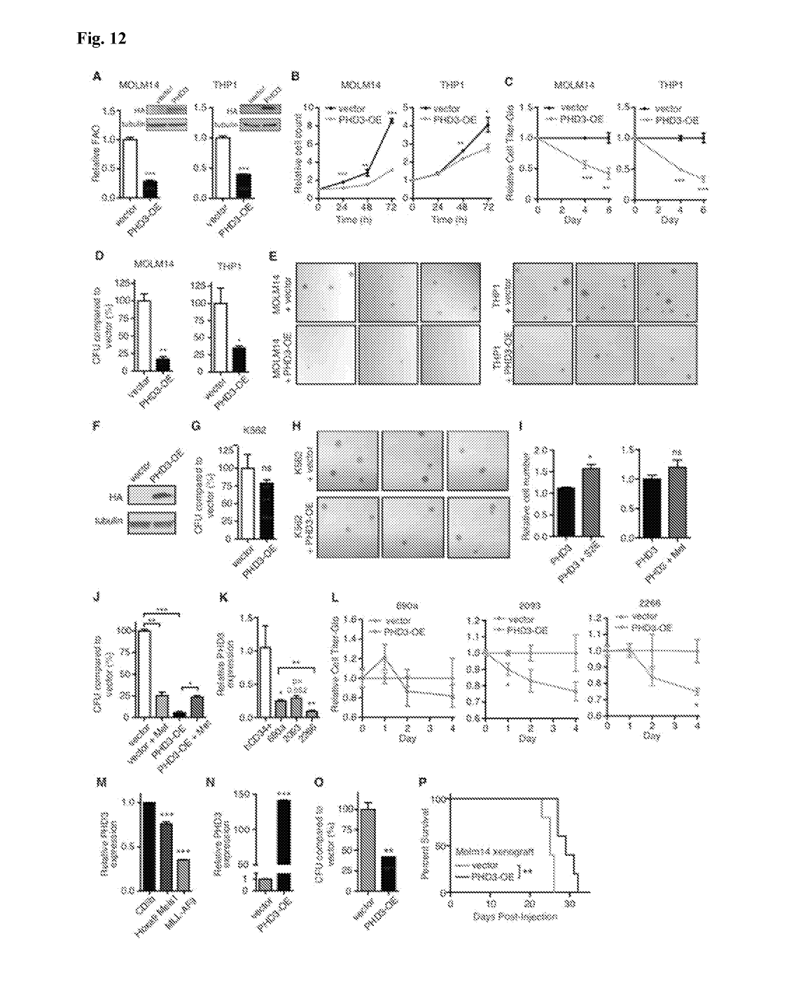

To characterize the dynamic nature of PHD3 response to nutrients and ACC hydroxylation, a time course analysis of ACC2 hydroxylation under high versus low nutrient conditions was performed. It was found that in response to nutrient abundance, PHD3 dramatically altered ACC2 hydroxylation within minutes. ACC2 hydroxylation was strongly decreased following 6 h in low glucose, serum-free medium, and hydroxylation increased after only 10 minutes of returning cells to high nutrient medium (FIG. 10, Panel D). Furthermore, this process was PHD3-dependent. PHD3 silencing most potently repressed ACC2 activity in the time frame immediately after restoring high nutrients to MEFs (FIG. 13, Panel J and FIG. 12, Panel K). Thus, this data suggest that PHD3 is a rapidly triggered metabolic toggle that represses FAO in response to cellular nutrient abundance.

It was reasoned that, in cells with low PHD3, this metabolic switch would be lacking. In multiple cell lines, palmitate oxidation was enhanced in serum-free, low glucose medium but blunted in the presence of high glucose and serum (FIGS. 10, Panel E and 13, Panel L). However, when PHD3 levels were reduced, cells lost sensitivity to this nutrient switch and displayed consistently elevated FAO even in the presence of high nutrients. Similarly, supplementing low nutrient medium with a cell-permeable version of the TCA cycle intermediate ca-ketoglutarate repressed palmitate oxidation in a PHD3-dependent manner (FIG. 10, Panel F). These data indicate that PHD3 limits FAO in nutrient-replete conditions, and that nutrient deprivation lifts PHD3-mediated repression of FAO.

These findings support a model in which PHD3 activates ACC2 to inhibit CPT1 and repress fatty acid catabolism (FIG. 10, Panel H). In support of this mechanism, metabolomics analysis revealed that long chain acylcarnitines, which are generated by CPT1, were elevated following PHD3 knockdown, but short and medium chain acylcarnitines, which bypass the ACC2/CPT1 regulatory node, were unchanged (FIG. 10, Panel G). The data additionally suggest that PHD3 regulation of FAO may function in parallel with AMPK (FIG. 10, Panel H). On one hand, as the bioenergetic rheostat of the cell, AMPK inhibits ACC2 under low-bioenergetic conditions to shift the cell toward higher FAO. This process is inherently sensitive to the cellular AMP/ATP ratio. PHD3 adds a complementary layer of control by activating ACC2 under high nutrient conditions, thereby repressing FAO and allowing fatty acids to be preserved for later use. Together, AMPK and PHD3 toggle FAO in a manner that is sensitive to both high and low nutrient levels (FIG. 10, Panel H).

### Example 8—Low PHD3 Expression Drives Altered Metabolism in AML

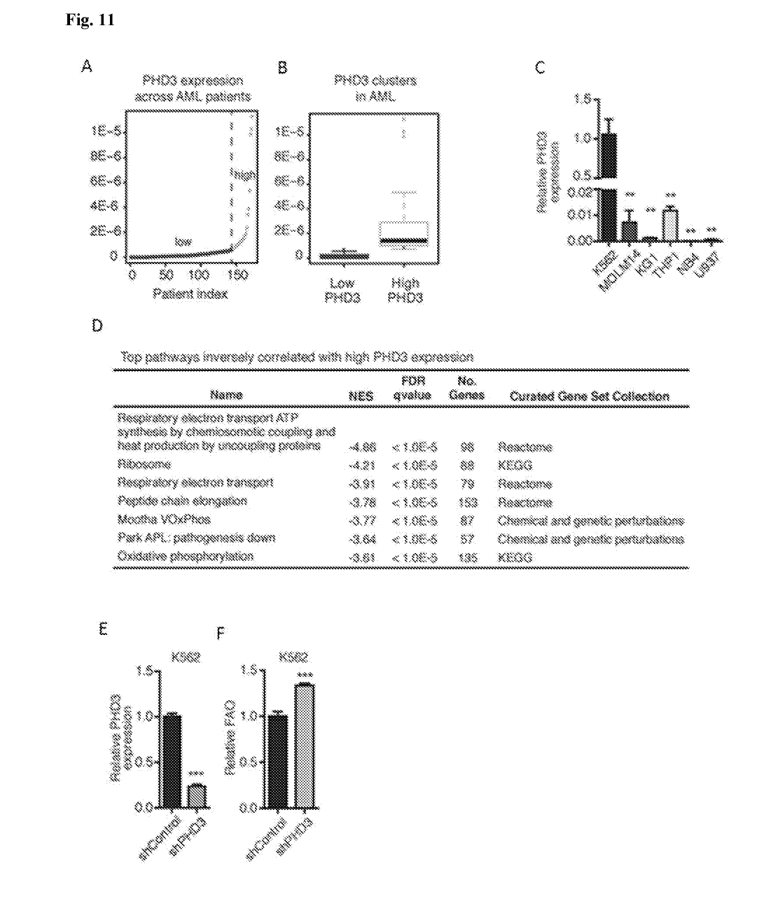

Whether low PHD3 expression might indicate elevated FAO and an altered metabolic state in patients with AML was probed. Using gene expression data from patient samples profiled as part of The Cancer Genome Atlas (TCGA), AML patients were clustered into two groups (PHD3-low and PHD3-high) using a Gaussian mixture model based on the level of PHD3 expression. Nearly 80% of patients fell into the low PHD3 group (FIG. 11, Panels A and B). Gene Set Enrichment Analysis was used to query cellular pathways linked with PHD3 expression in AML patients. This analysis revealed that the top curated gene sets inversely correlated with high PHD3 expression in AML are largely markers of oxidative metabolism (FIG. 11, Panel C, box plots of individual gene sets in FIG. 14, Panels A-D). These include multiple gene sets involving the electron transport chain and oxidative phosphorylation. This suggests that, in AML, a high level of PHD3 expression may serve as an indicator that the cancer cells are not fueled by oxidative metabolism. Of note, no significant link between PHD3 expression and expression of ACC2, AMPK or LKB1 (FIG. 14, Panels E-G) was found in TCGA patient sample data. These data support a model in which low PHD3 expression in AML can enable a metabolic switch toward oxidative metabolism via altered ACC hydroxylation and function.

In line with patient data, PHD3 gene expression was nearly undetectable in a panel of AML cell lines (MOLM14, KG1, THP1, NB4 and U937) compared to the K562 chronic myeloid leukemia (CML) cell line (FIG. 11, Panel D). Low-PHD3 AML cells show reduced ACC hydroxylation and ATP binding (FIG. 4, Panels L and M) and markedly increased palmitate oxidation (FIG. 4, Panel e). PHD1 and PHD2 are not repressed to the same extent as PHD3 in AML cells (FIG. 14, Panels H and I), indicating that PHD3 expression is specifically linked to the observed metabolic traits. In high-PHD3 K562 cells, PHD3 knockdown enabled substantially higher FAO, demonstrating the consequence of PHD3 loss in leukemia cells (FIG. 11, Panels E and F).

Further, it was hypothesized that low-PHD3 leukemia cells possess a metabolic liability rooted in their dependency on FAO. Therefore leukemia cell sensitivity to etomoxir or ranolazine, FAO inhibitors that have shown success in treating angina and heart disease, was examined. Etomoxir represses FAO by inhibiting CPT1, and ranolazine inhibits 3-ketoacylthiolase, the enzyme catalyzing the final step in each round of β-oxidation. It was observed that 96 h inhibition of FAO led to substantial cell death in low-PHD3 leukemia cells, while viability was maintained for high-PHD3 K562 cells (FIG. 4, Panels H-J and FIG. 14, Panel J). Another high-PHD3 CML cell line, MEG01, was also less sensitive to a high dose of ranolazine compared to low-PHD3 AML cells (FIG. 14, Panels K and L). Sensitivity to FAO inhibition was more strongly linked to PHD3 status than to classification as AML or CML; a CML cell line with low PHD3 expression (KU812) was found to be sensitive to treatment with etomoxir and more closely resembled another low-PHD3 AML cell line (NB4) than a high-PHD3 CML line (K562) (FIG. 14, Panels J and L). Thus, blocking fatty acid catabolism has a strong cytotoxic effect particularly in low-PHD3 leukemia cells.

Although PHD3 knockdown in K562 cells enabled higher FAO (FIG. 11, Panel F), it did not create a fixed dependency on FAO or cause susceptibility to FAO inhibitors (FIG. 14, Panel M). K562 cells have a strong preference for glycolytic versus oxidative metabolism, and although PHD3 knockdown enabled higher FAO, it did not force these cells to rely on fatty acids. In contrast, low-PHD3 cancer cells do indeed display limited metabolic plasticity, require sustained FAO and are particularly susceptible to pharmacological inhibitors of FAO. Thus, these data indicate that low PHD3 expression may be a candidate as a biomarker for leukemia cells that may be successfully targeted with FAO inhibitors.

### Example 9—Restoring PHD3 Expression in AML Limits Cancer Cell Growth and Leukemogenic Potential

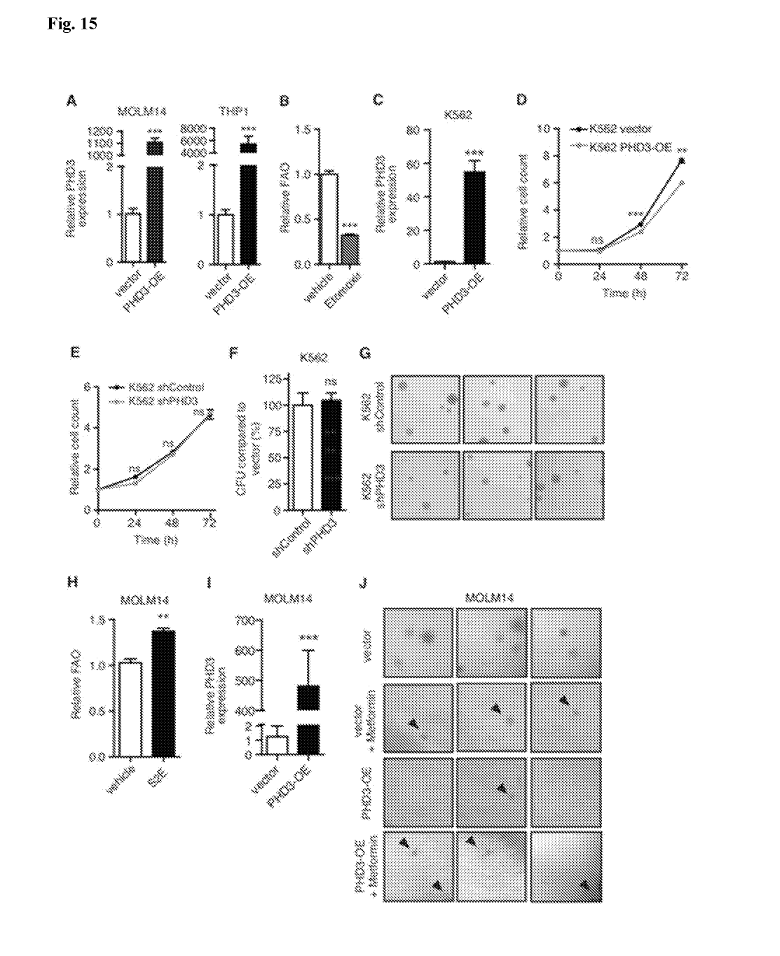

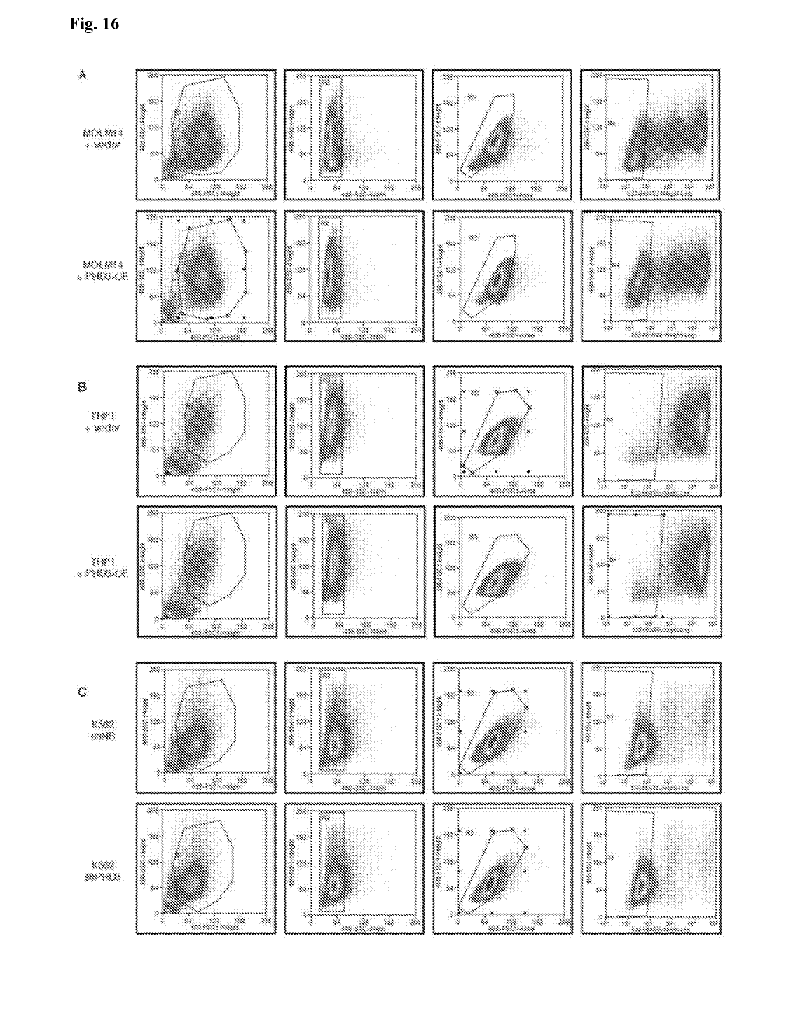

The data suggest that low PHD3 expression is advantageous in AML by enabling increased FAO, a metabolic program vital for growth and proliferation in this cancer setting. Thus, restoring PHD3 levels would limit the proliferation and potency of existing leukemia cells. To this end, the consequences of PHD3 overexpression in the low-PHD3 AML cell lines, MOLM14 and THP1 was examined. Stable PHD3 overexpression repressed FAO by over 50% (FIG. 12, Panel A and FIG. 15, Panel A), matching a level similar to that achieved by etomoxir (FIG. 15, Panel B). This suggests that PHD3 affects FAO at a magnitude similar to what is achieved by direct CPT1 inhibition. Stable PHD3 overexpression in low-PHD3 AML cells also diminished cell proliferation and viability (FIGS. 12, Panel B and 5, Panel C, FACS plots of sorted cells in FIG. 16, Panels A and B). Furthermore, the impact of PHD3 overexpression on leukemia potency was probed using colony formation assays to measure viable and functional progenitor cells. Overexpressing PHD3 dramatically decreased the number of clonogenic MOLM14 and THP1 cells in methylcellulose assays (FIGS. 12, Panel D and 12, Panel E).

To assess whether PHD3 overexpression was generally toxic, PHD3 was overexpressed in K562 cells and examined the effect on growth. Endogenous PHD3 levels in MOLM14 and THP1 cells are 1% of that in K562 cells (FIG. 11, Panel D), and PHD3 overexpression on the order of 1000 to 6000-fold in these cells achieves an amount roughly 10 to 60-fold greater than that found in K562 cells (FIG. 15, Panel A). To assess the toxicity of this amount of PHD3, PHD3 was overexpressed in K562 cells by 60-fold (FIG. 15, Panel C). This level of PHD3 overexpression had only a subtle effect on K562 cell proliferation (FIG. 15, Panel D; HA-PHD3 overexpression in FIG. 12, Panel F) and, more notably, did not impact growth in colony formation assays (FIGS. 12, Panel G and 12, Panel H). Thus, these data indicate the growth inhibitory effects of PHD3 overexpression are specific to low-PHD3 AML cells and are not generally toxic to all cells. Moreover, modulating PHD3 in the opposite direction, via stable knockdown by shRNA, also did not alter proliferation or clonogenic capacity in high-PHD3 K562 cells (FIG. 15, Panels E-G, FACS plots of sorted cells in FIG. 16, Panel C). This supports the idea that the metabolic alterations due to modulating PHD3 are not detrimental to all leukemia cells. Instead, low-PHD3 leukemia cells in particular experience severe detrimental effects when PHD3 is restored.

This model suggests that PHD3 overexpression is harmful to low-PHD3 AML cells by activating ACC2 and thereby repressing fatty acid catabolism. To test this model, whether PHD3 overexpression would still have a strong effect on AML when ACC2 is inhibited using previously described and validated small molecules was assessed. First, it was found that the ACC inhibitor S2E amplified FAO as expected (FIG. 15, Panel H). Importantly, S2E treatment rescued Molm14 cell growth at 72 h following PHD3 overexpression (FIG. 12, Panel I). Furthermore, treatment with metformin, reported to repress ACC via activation of AMPK, caused a trend toward rescued growth and also improved growth of PHD3-overexpressing Molm14 cells in colony formation assays (FIG. 12, Panels I-J and FIG. 15, Panel I-J). Interestingly, while metformin on its own impaired cell growth in soft agar, metformin partially rescues AML cells from FAO inhibition that is caused in this case by overexpressing PHD3 (FIG. 12, Panel J). Overall, it was observed that PHD3 overexpression was most detrimental to low-PHD3 AML cells when ACC2, a key component of the cellular mechanism identified here, was available to be activated.

Next, whether PHD3 overexpression also inhibits proliferation in primary AML samples was determined. Consistent with the bioinformatics analysis, leukemic cells from patient samples obtained from the University of Pennsylvania showed decreased PHD3 expression compared to healthy CD34⁺ control bone marrow cells (FIG. 12, Panel K). Overexpressing PHD3, but not empty vector, decreased cell proliferation in 2 of the 3 patient samples, while the remaining sample trended toward a decrease (FIG. 12, Panel L). PHD3 overexpression led to similar results in leukemic cells derived from the MLL/AF9 mouse model of AML. MLL/AF9 chromosomal translocation is a causative factor in a substantial subset of human AML and is associated with a 5-year survival rate of only 40%. Compared to healthy CD11b control cells, PHD3 was strongly decreased in leukemic cells obtained from the MLL/AF9 mouse model of AML and decreased to a lesser extent in the Hoxa9 Meis1 mouse model of AML (FIG. 12, Panel M). In MLL-AF9 lineage-negative bone marrow cells, PHD3 overexpression decreased AML clonogenic capacity (FIGS. 12, Panel N and 12, Panel O). Thus, in low-PHD3 systems, PHD3 overexpression limits leukemic potency.

Finally, the in vivo impact of PHD3 overexpression in low-PHD3 AML cells was evaluated using a mouse xenotransplanation model. NOD-scid IL2Rgamma^{null} (NSG) mice were chosen for this analysis due to their superiority in allowing engraftment of human AML cells. Cohorts of NSG mice were injected via tail vein with MOLM14 cells overexpressing PHD3 or vector. The length of survival post-injection was used as a readout of AML severity. It was observed that PHD3 overexpression in AML enhanced survival (FIG. 12, Panel P). Taken together, these new data suggest that low-PHD3 leukemia cells possess a metabolic liability rooted in ACC2 activation and a dependency on FAO, and that restoring PHD3 levels limits the proliferation and potency of AML cells.

### Example 10. Materials and Methods

Reagents and Constructs.

For transient overexpression studies, Fugene 6 (Roche) was used to transfect 293T cells. ARNT −/− mouse hepatoma cells were transfected with Fugene D. pcDNA3.1 empty vector and constructs containing HA-PHD1, PHD2 and PHD3 were previously described[53]. HA-PHD3 pcDNA 3.1 point mutants were generated using the QuikChange II Site-Directed Mutagenesis Kit (Agilent). ACC2 cDNA in pENTR223 vector was obtained from the Dana Farber/Harvard Cancer Center Resource Core. For transient overexpression, ACC2 cDNA was cloned into pDEST vector (Wader Harper lab at Harvard Medical School) using Gateway LR Clonase II Enzyme Mix according to manufacturer's instructions. Briefly, 10 μl reactions containing 150 ng ACC2 pENTR223, 150 ng pDEST vector and 2 μl Clonase in TE buffer (pH 8.0) were incubated at 25° for 2 hr. ACC2 point mutants were generated using the QuikChange II XL Site-Directed Mutagenesis Kit (Agilent). Mutagenesis primers are listed below. For stable overexpression via retroviral infection, the HA-PHD3 construct was cloned from pCDNA3.1 into the pBABE puro vector.

MOLM14 cells were retrovirally infected via spin infection. 300,000 cells were resuspended in 2 ml of complete media supplemented with polybrene, and 500 μl virus was added. Cells were centrifuged at 37° C. for 1 hr at 2250 rpm, then re-plated in fresh media in a 6-well plate.

For transient knockdown, cells were transfected with 22.5 nM siRNA and Dharmafect 1 Transfection Reagent (Dharmacon) according to manufacturer's instructions. Cells were transfected with siGENOME SMARTpool EGLN3 siRNA or control Non-Targeting siRNA Pool #2 (Dharmacon).

For stable knockdown, lentiviral shRNA against PHD3 were obtained from The RNAi Consortium at the Broad Institute/Harvard. pLKO empty vector was used as non-silencing control. Stable knockdown cell lines were generated following the Consortium instructions. Target sequences for shRNA are listed below. In experiments using one shRNA against PHD3, shPHD3.2 was used.

Primers for Mutagenesis

| Point mutant | Primer |
|---|---|
| ACC2 P450A | (F) AGAAGCTTTGATCATCAATGCAAAACCAATTCTTTCTGCTGC |
| | (SEQ ID NO: 19) |
| | (R) GCAGCAGAAAGAATTGGTTTTGCATTGATGATCAAAGCTTCT |
| | (SEQ ID NO: 20) |
| ACC2 P343A | (F) CCGCCTGCACGGCGATTCTCTTGGC (SEQ ID NO: 21) |
| | (R) GCCAAGAGAATCGCCGTGCAGGCGG (SEQ ID NO: 22) |
| ACC2 P2131A | (F) GTAGGCTGAGTCTGCGAACCACACCTGTC (SEQ ID NO: 23) |
| | (R) GACAGGTGTGGTTCGCAGACTCAGCCTAC (SEQ ID NO: 24) |
| ACC2 P450G | (F) GGCAGCAGAAAGAATTGGTTTTGGATTGATGATCAAAGCTTC TGA |
| | (SEQ ID NO: 25) |
| | (R) TCAGAAGCTTTGATCATCAATCCAAAACCAATTCTTTCTGCTGCC |
| | (SEQ ID NO: 26) |
| PHD3 H196A | (F) CAGATCGTAGGAACCCAGCCGAAGTGCAGCCCT (SEQ ID NO: 27) |
| | (R) AGGGCTGCACTTCGGCTGGGTTCCTACGATCTG (SEQ ID NO: 28) |
| PHD3 R206K | (F) GCCCTCTTACGCAACCAAATATGCTATGACTGTCT |
| | (SEQ ID NO: 29) |
| | (R) AGACAGTCATAGCATATTTGGTTGCGTAAGAGGGC (SEQ ID NO: |
| | 30) |

shRNA Target Sequences

| Name | Clone ID | Target Sequence |
|---|---|---|
| shPHD3.1 | TRCN0000001048 | CACCTGCATCTACTATCTGAA |
| | | (SEQ ID NO: 31) |
| shPHD3.2 | TRCNO000001050 | GTGGCTTGCTATCCGGGAAAT |
| | | (SEQ ID NO: 32) |

Cell Culture.

293T cells and 786-O VHL^{-/-} cells were cultured in 4.5 g/L glucose DMEM (Invitrogen) supplemented with 10% FBS and penicillin/streptomycin. Low glucose DMEM contained 1 g/L glucose. ARNT-deficient mouse hepatoma c4 (B13NBii1) cells previously derived from Hepa clc7 cells were cultured in Minimum Essential Media alpha (Invitrogen) supplemented with 10% heat-inactivated FBS and penicillin/streptomycin. K562, MOLM14, THP1, KU812 and NB4 cells were maintained in RPMI 1640 media (Invitrogen) supplemented with 10% FBS and penicillin/streptomycin. KG1 cells were maintained in IMDM (Invitrogen) supplemented with 20% FBS and penicillin/streptomycin. HepG2 cells were cultured in Minimum Essential Medium Eagle (Sigma) supplemented with 10% FBS, penicillin/streptomycin, 1% sodium pyruvate and 1% non-essential amino acids. All cell lines were tested with the Universal Mycoplasma Detection Kit (ATCC) to ensure absence of mycoplasma.

Quantitative RT-PCR Analysis.

RNA was isolated by extraction with Trizol according to manufacturer instructions (Invitrogen). cDNA was synthesized using iScript cDNA synthesis kit (BioRad). Quantitative real-time PCR was performed with Sybr Green I Mastermix (Roche) or Sybr Green Fast Mix (Quanta Biosciences) on a Roche Lightcycler 480 and analyzed by using AACt calculations. qPCR analyses in human cell lines are relative to the reference gene B2M. qPCR analyses in mouse ARNT −/− hepatoma cell line are relative to RPS4X. Primer sequences are provided below.

| | |
|---|---|
| Gene | Primer |

Human

| | |
|---|---|
| PHD1 | (F) ACGGGCTCGGGTACGTAAG |
| | (SEQ ID NO: 33) |
| | (R) CCCAGTTCTGATTCAGGTAATAGATACA |
| | (SEQ ID NO: 34) |
| PHD2 | (F) GACCTGATACGCCACTGTAACG |
| | (SEQ ID NO: 35) |
| | (R) CCCGGATAACAAGCAACCAT |
| | (SEQ ID NO: 36) |
| PHD3 | (F) ATACTACGTCAAGGAGAGGT |
| | (SEQ ID NO: 37) |
| | (R) TCAGCATCAAAGTACCAGA |
| | (SEQ ID NO: 38) |
| B2M | (F) AGATGAGTATGCCTGCCGTGTGAA |
| | (SEQ ID NO: 39) |
| | (R) TGCTGCTTACATGTCTCGATCCCA |
| | (SEQ ID NO: 40) |
| ACC1 | (F) ATCCCGTACCTTCTTCTACTG |
| | (SEQ ID NO: 41) |
| | (R) CCCAAACATAAGCCTTCACTG |
| | (SEQ ID NO: 42) |
| ACC2 | (F) CTCTGACCATGTTCGTTCTC |
| | (SEQ ID NO: 43) |
| | (R) ATCTTCATCACCTCCATCTC |
| | (SEQ ID NO: 44) |
| CPT1a | (F) GATTTTGCTGTCGGTCTTGG |
| | (SEQ ID NO: 45) |
| | (R) CTCTTGCTGCCTGAATGTGA |
| | (SEQ ID NO: 46) |
| CPT1b | (F) ATTCCCACCGCGGAAGGTGC |
| | (SEQ ID NO: 47) |
| | (R) GCAGCCTGGGGGCAGTCTTG |
| | (SEQ ID NO: 48) |
| ACADM | (F) TCATTGTGGAAGCAGATACCC |
| | (SEQ ID NO: 49) |
| | (R) CAGCTCCGTCACCAATTAAAAC |
| | (SEQ ID NO: 50) |
| LIPG | (F) TGTGGAAGGAGTTTCGCAG |
| | (SEQ ID NO: 51) |
| | (R) GGGATATGCTGGTGTTCTCAG |
| | (SEQ ID NO: 52) |
| PGK1 | (F) CCACTTGCTGT GCCAAATGGA |
| | (SEQ ID NO: 53) |
| | (R) GAAGGACTTTACCTTCCAGGA |
| | (SEQ ID NO: 54) |
| HK2 | (F) GATTGTCCGTAACATTCTCATCGA |
| | (SEQ ID NO: 55) |
| | (R) TGTCTTGAGCCGCTCTGAGAT |
| | (SEQ ID NO: 56) |

Mouse

| | |
|---|---|
| RPS4X | (F) ACCCTGCTGGGTTTATGGATGTCA |
| | (SEQ ID NO: 57) |
| | (R) TACGATGAACAGCAAAGCGACCCT |
| | (SEQ ID NO: 58) |
| PHD3 | (F) CAGACCGCAGGAATCCACAT |
| | (SEQ ID NO: 59) |
| | (R) TTCAGCATCGAAGTACCAGACAGT |
| | (SEQ ID NO: 60) |

Immunoprecipitation, Western Blotting and Antibodies.

Western blotting was performed using antibodies against ACC (Cell Signaling Technologies (CST) no. 3676), ACC1 isoform (CST no. 4190), ACC2 isoform (CST no. 8578), HA (CST no. 2367), hydroxyproline (Abcam no. ab37067), tubulin (Sigma no. T5168), HIF1α (BD no. 610959), HIF2α (CST no. 7096), a ctin (Sigma no. A2066), LSD1 (CST no. 2139) and PHD3 (Novus Biologicals no. NB100-139). For immunoprecipitations of transiently overexpressed HA-tagged proteins, lysates were immunoprecipitated using EZview anti-HA Affinity Gel (Sigma no. E6779). For endogenous immunoprecipitations, lysates were immunoprecipitated with ACC antibody (CST no. 3767) or ACC2 antibody (CST no. 8578) and EZview Red Protein G Affinity Gel (Sigma no. E3403).

Mass Spectrometry.

To identify hydroxylated proline sites, ACC2 was transiently overexpressed in 293T cells. 48 hours later, cell lysates were collected and ACC2 was immunoprecipitated with ACC2 antibody and Protein G Affinity Gel described above. Bound material was washed and separated by SDS-PAGE. The Coomassie stained band was excised, analyzed by LC-MS2 and searched against the Uniprot Human database (downloaded August 2011) using Sequest with proline hydroxylation set as a variable modification (+15.9949 molecular weight shift).

In Vitro Hydroxylation Assay.

The in vitro hydroxylation assay was modified from a previously described assay based on the fact that hydroxylation by PHDs results in decarboxylation of α-ketoglutarate to form carbon dioxide [56]. Briefly, 250 ml reactions were performed in glass vials sealed with rubber stoppers and parafilm wax. Reaction mixtures containing 12.5 nmol synthetic peptide (Peptide 2.0), 50 mM Tris/HCl (pH 7.8), 2 mg/ml BSA, 4200 U/ml catalase, 0.1 mM DTT, 2 mM ascorbate, 500 μM FeSO4.7H₂O, 0.02 μmol [1-¹⁴C]α-ketoglutarate (Perkin Elmer) and 1.2 μg recombinant PHD3 were incubated at 37° for 30 min. Reactions were stopped by injection of 0.25 ml of 1 M KH₂PO₄ (pH 5) into vials. Vials were agitated on slow speed for 30 min at room temperature to allow capture of [¹⁴C]CO₂ onto solubilized Whatman paper positioned at the top of the vials. CPM were measured by scintillation counts on filter paper.

Peptides for In Vitro Hydroxylation

| Proline | Sequence |
|---|---|
| 450 | ERIGFPLMIKASEGGGGK (SEQ ID NO: 61) |
| 2131 | AGQVWFPDSAYKTAQ (SEQ ID NO: 62) |
| 966 | ARLELDDPSKVHPAE (SEQ ID NO: 63) |

ACC Activity Assay.

Reactions were performed as previously described[57], with the exception of using 16.7 mM MgCl₂ instead. 50 μg protein lysate was used for each reaction. Following addition of 1 N HCl to quench reactions and convert remaining [¹⁴C]bicarbonate (American Radiolabeled Chemicals) to CO₂, reactions were evaporated 2 hours at 60° and 15 min at 85° in a thermo shaker. ACC activity was calculated as incorporation of [¹⁴C]bicarbonate into [¹⁴C]malonyl CoA (the acid and heat stable product) as measured by scintillation counting.

ATP Binding Assays.

ATP immunoprecipitations were performed using the ATP AffiPur Kit (Jena Bioscience), which contained aminophenyl-ATP-Agarose, C10-spacer. Procedure was done according to manufacturer's instructions, except for the following distinction. Transiently transfected 293T cells were lysed in ACC activity assay buffer[57] to promote native protein folding. Following dialysis to remove endogenous ATP and immunoprecipitation with ATP-affinity resin, bound material was washed and eluted by addition of sample buffer containing beta-mercaptoethanol. Samples were boiled 5 min at 95° for analysis by Western blot.

Fatty Acid Oxidation.

For FAO assays, cells in 12 well-plates were pre-incubated with 100 μM palmitate or hexanoate and 1 mM carnitine for 4 hr in serum-free low glucose media, unless otherwise noted. Cells were then changed to 600 μl media containing 1 μCi [9,10(n)-³H]palmitic acid (GE Healthcare) or 1.8 μCi n-[5,6-³H]hexanoic acid (American Radiolabeled Chemicals) and 1 mM carnitine for 2 hr. The medium was collected and eluted in columns packed with DOWEX 1X2-400 ion exchange resin (Sigma) to analyze the released ³H₂O, formed during oxidation of [³H]palmitate. FAO in complete media indicates media including serum was used for pre-incubation and FAO analysis. Basal FAO indicates cells were not pre-incubated with fatty acids prior to FAO analysis. For all FAO experiments, counts per minute (CPM) were normalized to protein content in cell lysates.

Lipogenesis.

Lipogenesis was performed as previously described[58] with the following modifications. Cells were pulsed for 4 hr with 4 μCi [¹⁴C]acetate±20 μM C75, then lipids were extracted. Scintillation counts were normalized to protein concentration in parallel plates.

Drug Treatment and PI Staining.

Cells were treated 96 hr with a range of doses of etomoxir (Cayman Chemical) or ranolazine dihydrochloride (VWR/Selleck Chemicals) or vehicle. Fresh etomoxir was spiked in at 24, 48 and 72 hr. Fresh ranolazine was spiked in at 48 hr. Dosing schedules were determined by identifying the minimum number of times drug must be re-added to observe an effect on cell viability. Cell viability at 96 hr was determined by staining cells with 1 μg/ml propidium iodide (Sigma) in PBS and flow cytometry on the BD LSR-II analyzer.

Growth Rates.

MOLM14 cells were plated in the wells of a 24 well plate (50,000 cells/well). At indicated times, cells were counted on the Beckman Z 1 Coulter Counter.

### Molecular Modeling.

Using CCP4 mg molecular graphic software, the biotin-carboxylase domain of human ACC2 (PDB: 3JRW) was superposed with the e. coli biotin-carboxylase domain bound to ATP (PDB: 1DV2) to highlight the likely position of ATP in the catalytic site of human ACC2.

Statistical Analysis.

Unpaired two-tailed Student's t tests were used. All experiments were performed at least two to three times.

### REFERENCES

1. Kaelin, W. G., Jr. & Ratcliffe, P. J. Oxygen Sensing by Metazoans: The Central Role of the HIF Hydroxylase Pathway. Molecular Cell 30, 393-402 (2008).
2. Cantor, J. R. & Sabatini, D. M. Cancer Cell Metabolism: One Hallmark, Many Faces. Cancer Discovery 2, 881-898 (2012).
3. Gorres, K. L. & Raines, R. T. Prolyl 4-hydroxylase. Critical Reviews in Biochemistry and Molecular Biology 45, 106-124 (2010).
4. DeBerardinis, R. J., Lum, J. J., Hatzivassiliou, G. & Thompson, C. B. The Biology of Cancer: Metabolic Reprogramming Fuels Cell Growth and Proliferation. Cell Metabolism 7, 11-20 (2008).
5. Carracedo, A., Cantley, L. C. & Pandolfi, P. P. Cancer metabolism: fatty acid oxidation in the limelight. Nat Rev Cancer 13, 227-232 (2013).
6. Tong, L. Acetyl-coenzyme A carboxylase: crucial metabolic enzyme and attractive target for drug discovery. Cellular and Molecular Life Sciences (CMLS) 62, 1784-1803 (2005).
7. Ward, P. S. & Thompson, C. B. Metabolic Reprogramming: A Cancer Hallmark Even Warburg Did Not Anticipate. Cancer Cell 21, 297-308 (2012).
8. Mullen, A. R. & DeBerardinis, R. J. Genetically-defined metabolic reprogramming in cancer. Trends in Endocrinology & Metabolism 23, 552-559 (2012).
9. Wallace, D. C. Mitochondria and cancer. Nat Rev Cancer 12, 685-698 (2012).
10. Finley, L. W. S. et al. SIRT3 Opposes Reprogramming of Cancer Cell Metabolism through HIF1&alpha; Destabilization. Cancer Cell 19, 416-428 (2011).
11. Losman, J. A. & Kaelin, W. G. What a difference a hydroxyl makes: mutant IDH, (R)-2-hydroxyglutarate, and cancer. Genes & Development 27, 836-852 (2013).
12. Masson, N. & Ratcliffe, P. J. Hypoxia signaling pathways in cancer metabolism: the importance of co-selecting interconnected physiological pathways. Cancer Metab 2, 3 (2014).
13. Vander Heiden, M. G. et al. Metabolic Pathway Alterations that Support Cell Proliferation. Cold Spring Harbor Symposia on Quantitative Biology 76, 325-334 (2012).
14. Luo, W. et al. Pyruvate Kinase M2 Is a PHD3-Stimulated Coactivator for Hypoxia-Inducible Factor 1. Cell 145, 732-744 (2011).
15. Berra, E. et al. HIF prolyl-hydroxylase 2 is the key oxygen sensor setting low steady-state levels of HIF-1α in normoxia. The EMBO Journal 22, 4082-4090 (2003).
16. Wong, B. W., Kuchnio, A., Bruning, U. & Carmeliet, P. Emerging novel functions of the oxygen-sensing prolyl hydroxylase domain enzymes. Trends in Biochemical Sciences 1-9 (2012). doi:10.1016/j.tibs.2012.10.004
17. Myllyharju, J. & Koivunen, P. Hypoxia-inducible factor prolyl 4-hydroxylases: common and specific roles. Biological Chemistry 394,
18. Smirnova, N. A. et al. Catalytic mechanism and substrate specificity of HIF prolyl hydroxylases. Biochemistry Moscow 77, 1108-1119 (2012).
19. Brownsey, R. W., Boone, A. N., Elliott, J. E., Kulpa, J. E. & Lee, W. M. Regulation of acetyl-CoA carboxylase. Biochem. Soc. Trans. 34, 223-227 (2006).
20. Saddik, M., Gamble, J., Witters, L. A. & Lopaschuk, G. D. Acetyl-CoA carboxylase regulation of fatty acid oxidation in the heart. J. Biol. Chem. 268, 25836-25845 (1993).
21. Abu-Elheiga, L., Oh, W., Kordari, P. & Wakil, S. J. Acetyl-CoA carboxylase 2 mutant mice are protected against obesity and diabetes induced by high-fat/high-carbohydrate diets. Proc. Natl. Acad. Sci. U.S.A. 100, 10207-10212 (2003).
22. Fullerton, M. D. et al. Single phosphorylation sites in Acc1 and Acc2 regulate lipid homeostasis and the insulin-sensitizing effects of metformin. Nat Med 19, 1649-1654 (2013).
23. Yoon, M. J. Adiponectin Increases Fatty Acid Oxidation in Skeletal Muscle Cells by Sequential Activation of AMP-Activated Protein Kinase, p38 Mitogen-Activated Protein Kinase, and Peroxisome Proliferator-Activated Receptor. Diabetes 55, 2562-2570 (2006).
24. Laurent, G. et al. SIRT4 Coordinates the Balance between Lipid Synthesis and Catabolism by Repressing Malonyl CoA Decarboxylase. Molecular Cell 50, 686-698 (2013).
25. Gerhart-Hines, Z. et al. Metabolic control of muscle mitochondrial function and fatty acid oxidation through SIRT1/PGC-1alpha. The EMBO Journal 26, 1913-1923 (2007).
26. Bruick, R. K. A Conserved Family of Prolyl-4-Hydroxylases That Modify HIF. Science 294, 1337-1340 (2001).
27. Rantanen, K. et al. Prolyl Hydroxylase PHD3 Activates Oxygen-dependent Protein Aggregation. Molecular Biology of the Cell 19, 2231-2240 (2008).
28. Loenarz, C. & Schofield, C. J. Physiological and biochemical aspects of hydroxylations and demethylations catalyzed by human 2-oxoglutarate oxygenases. Trends in Biochemical Sciences 36, 7-18 (2011).
29. Almarza-Ortega, D. B. Human Acetyl-CoA Carboxylase 2. MOLECULAR CLONING, CHARACTERIZATION, CHROMOSOMAL MAPPING, AND EVIDENCE FOR TWO ISOFORMS. Journal of Biological Chemistry 272, 10669-10677 (1997).
30. Ruderman, N. & Prentki, M. AMP kinase and malonyl-CoA: targets for therapy of the metabolic syndrome. Nat Rev Drug Discov 3, 340-351 (2004).
31. Cho, Y. S. et al. Molecular mechanism for the regulation of human ACC2 through phosphorylation by AMPK. Biochemical and Biophysical Research Communications 391, 187-192 (2010).
32. Thoden, J. B. Movement of the Biotin Carboxylase B-domain as a Result of ATP Binding. Journal of Biological Chemistry 275, 16183-16190 (2000).
33. Caro, P. et al. Metabolic Signatures Uncover Distinct Targets in Molecular Subsets of Diffuse Large B Cell Lymphoma. Cancer Cell 22, 547-560 (2012).
34. Samudio, I. et al. Pharmacologic inhibition of fatty acid oxidation sensitizes human leukemia cells to apoptosis induction. J. Clin. Invest. 120, 142-156 (2010).
35. Ramaswamy, S. et al. Multiclass cancer diagnosis using tumor gene expression signatures. Proc. Natl. Acad. Sci. U.S.A. 98, 15149-15154 (2001).
36. Valk, P. J. M. et al. Prognostically useful gene-expression profiles in acute myeloid leukemia. N. Engl. J. Med. 350, 1617-1628 (2004).
37. Andersson, A. et al. Microarray-based classification of a consecutive series of 121 childhood acute leukemias: prediction of leukemic and genetic subtype as well as of minimal residual disease status. Leukemia 21, 1198-1203 (2007).
38. Holubarsch, C. J. F. et al. A double-blind randomized multicentre clinical trial to evaluate the efficacy and safety of two doses of etomoxir in comparison with placebo in patients with moderate congestive heart failure: the ERGO (etomoxir for the recovery of glucose oxidation) study. Clin. Sci. 113, 205-212 (2007).
39. Nash, D. T. & Nash, S. D. Ranolazine for chronic stable angina. Lancet 372, 1335-1341 (2008).
40. Schafer, Z. T. et al. Antioxidants and metabolic defects Nature 2009. Nature 461, 109-113 (2009).
41. Jeon, S.-M., Chandel, N. S. & Hay, N. AMPK regulates NADPH homeostasis to promote tumour cell survival during energy stress. Nature 485, 661-665 (2012).
42. Zaugg, K. et al. Carnitine palmitoyltransferase 1C promotes cell survival and tumor growth under conditions of metabolic stress. Genes & Development 25, 1041-1051 (2011).

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

What is claimed is:

1. A method for treating a subject having a cancer comprising cancer cells with reduced PHD3 expression, the method comprising administering to the subject a fatty acid oxidation (FAO) inhibitor in an amount effective to treat the cancer.

2. A method for treating a subject having a cancer, the method comprising administering to the subject a fatty acid oxidation (FAO) inhibitor in an amount effective to treat the cancer, wherein the cancer has been identified as comprising cancer cells with reduced PHD3 expression.

3. A method for treating a subject having a cancer, the method comprising:
receiving the results of a test determining that the subject's cancer comprises cancer cells with reduced PHD3 expression; and
ordering administration of an effective amount of a fatty acid oxidation (FAO) inhibitor to the subject.

4. A method for treating a subject having a cancer, the method comprising:
requesting a test, or the results of a test, determining that the subject's cancer comprises cancer cells with reduced PHD3 expression; and
ordering administration of an effective amount of a fatty acid oxidation (FAO) inhibitor to the subject.

5. The method according to any one of claims 1 to 4, wherein the cancer is a prostate cancer.

6. The method according to any one of claims 1 to 4, wherein the cancer is a glioblastoma.

7. The method according to any one of claims 1 to 4, wherein the cancer is of hematological origin.

8. The method according to any one of claims 1 to 7, wherein the subject is a human.

9. The method according to any one of claims 1 to 8, wherein PHD3 expression by the cancer cells is less than or equal to 90% of normal cells of the same histological type from which the cancer cells are derived.

10. The method according to any one of claims 1 to 8, wherein PHD3 expression by the cancer cells is less than or equal to 80% of normal cells of the same histological type from which the cancer cells are derived.

11. The method according to any one of claims 1 to 8, wherein PHD3 expression by the cancer cells is less than or equal to 70% of normal cells of the same histological type from which the cancer cells are derived.

12. The method according to any one of claims 1 to 8, wherein PHD3 expression by the cancer cells is less than or equal to 50% of normal cells of the same histological type from which the cancer cells are derived.

13. The method according to any one of claims 1 to 8, wherein PHD3 expression by the cancer cells is less than or equal to 25% of normal cells of the same histological type from which the cancer cells are derived.

14. The method according to any one of claims 1 to 8, wherein PHD3 expression by the cancer cells is less than or equal to 15% of normal cells of the same histological type from which the cancer cells are derived.

15. The method according to any one of claims 1 to 14, further comprising determining whether the cancer cells have reduced PHD3 expression.

16. The method according to any one of claims 1 to 15, wherein the FAO inhibitor is a carnitine palmitoyl transferase (CPT-I) inhibitor.

17. The method according to claim 16, wherein the CPT-I inhibitor is etomoxir, oxfenicine, or perhexiline.

18. The method according to any one of claims 1 to 15, wherein the FAO inhibitor is a 3-ketoacyl-coenzyme A thiolase (3-KAT) inhibitor.

19. The method according to claim 18, wherein the 3-KAT inhibitor is trimetazidine or ranolazine.

20. The method according to any one of claims 1 to 15, wherein the FAO inhibitor is a mitochondrial thiolase inhibitor.

21. The method according to claim 20, wherein the mitochondrial thiolase inhibitor is 4-bromocrotonic acid.

22. A method for treating a subject having a cancer comprising cancer cells with elevated PHD3 expression, the method comprising administering to the subject a glycolytic pathway inhibitor in an amount effective to treat the cancer.

23. A method for treating a subject having a cancer, the method comprising administering to the subject a glycolytic pathway inhibitor in an amount effective to treat the cancer, wherein the cancer has been identified as comprising cancer cells with elevated PHD3 expression.

24. A method for treating a subject having a cancer, the method comprising:
receiving the results of a test determining that the subject's cancer comprises cancer cells with reduced PHD3 expression; and
ordering administration of an effective amount of a glycolytic pathway inhibitor to the subject.

25. A method for treating a subject having a cancer, the method comprising:
requesting a test, or the results of a test, determining that the subject's cancer comprises cancer cells with elevated PHD3 expression; and
ordering administration of an effective amount of a glycolytic pathway inhibitor to the subject.

26. The method according to any one of claims 22 to 25, wherein the cancer is a pancreatic cancer.

27. The method according to any one of claims 22 to 25, wherein the cancer is a kidney cancer or bladder cancer.

28. The method according to any one of claims 22 to 25, wherein the cancer is a melanoma, a lung cancer, a follicular lymphoma, a breast cancer, a colorectal cancer, or an ovarian cancer.

29. The method according to any one of claims 22 to 28, wherein the subject is a human.

30. The method according to any one of claims 22 to 29, wherein PHD3 expression by the cancer cells is at least 20% greater than that of normal cells of the same histological type from which the cancer cells are derived.

31. The method according to any one of claims 22 to 29, wherein PHD3 expression by the cancer cells is at least 50% greater than that of normal cells of the same histological type from which the cancer cells are derived.

32. The method according to any one of claims 22 to 29, wherein PHD3 expression by the cancer cells is at least 75% greater than that of normal cells of the same histological type from which the cancer cells are derived.

33. The method according to any one of claims 22 to 29, wherein PHD3 expression by the cancer cells is at least 100% greater than that of normal cells of the same histological type from which the cancer cells are derived.

34. The method according to any one of claims 22 to 29, wherein PHD3 expression by the cancer cells is at least 2.5 fold greater than that of normal cells of the same histological type from which the cancer cells are derived.

35. The method according to any one of claims 22 to 29, wherein PHD3 expression by the cancer cells is at least 5 fold greater than that of normal cells of the same histological type from which the cancer cells are derived.

36. The method according to any one of claims 22 to 35, further comprising determining whether the cancer cells have elevated PHD3 expression.

37. The method according to any one of claims 22 to 36, wherein the glycolytic pathway inhibitor is a hexokinase inhibitor.

38. The method according to claim 37, wherein the hexokinase inhibitor is 2-deoxyglucose.

39. The method according to claim 37, wherein the hexokinase inhibitor is 3-bromopyruvate.

40. The method according to claim 37, wherein the hexokinase inhibitor is lonidamine.

41. The method according to any one of claims 22 to 36, wherein the glycolytic pathway inhibitor is a transketolase inhibitor.

42. The method according to claim 41, wherein the transketolase inhibitor is oxythiamine.

43. The method according to any one of claims 22 to 36, wherein the glycolytic pathway inhibitor is imatinib.

44. The method according to any one of claims 22 to 36, wherein the glycolytic pathway inhibitor is a glucose transporter (GLUT) inhibitor.

45. The method according to any one of claims 22 to 36, wherein the glycolytic pathway inhibitor is a phosphofructokinase (PFK) inhibitor.

46. The method according to any one of claims 22 to 36, wherein the glycolytic pathway inhibitor is a glyceraldehyde-3-phosphate dehydrogenase (GAPDH) inhibitor.

47. The method according to any one of claims 22 to 36, wherein the glycolytic pathway inhibitor is a pyruvate kinase (PK) inhibitor.

48. The method according to any one of claims 22 to 36, wherein the glycolytic pathway inhibitor is a lactate dehydrogenase (LDH) inhibitor.

49. A method for treating a subject having a cancer characterized by cancer cells having a reduced level of hydroxylation of ACC2 at proline 450 relative to SEQ ID NO:2, the method comprising administering to the subject a fatty acid oxidation (FAO) inhibitor in an amount effective to treat the cancer.

50. A method for treating a subject having a cancer, the method comprising administering to the subject a fatty acid oxidation (FAO) inhibitor in an amount effective to treat the cancer, wherein the cancer has been identified as comprising cancer cells having a reduced level of hydroxylation of ACC2 at proline 450 relative to SEQ ID NO:2.

51. A method for treating a subject having a cancer, the method comprising:
receiving the results of a test determining that the subject's cancer comprises cancer cells having a reduced level of hydroxylation of ACC2 at proline 450 relative to SEQ ID NO:2; and
ordering administration of an effective amount of a fatty acid oxidation (FAO) inhibitor to the subject.

52. A method for treating a subject having a cancer, the method comprising:
requesting a test, or the results of a test, determining that the subject's cancer comprises cancer cells having a reduced level of hydroxylation of ACC2 at proline 450 relative to SEQ ID NO:2; and
ordering administration of an effective amount of a fatty acid oxidation (FAO) inhibitor to the subject.

53. The method according to any one of claims 49 to 52, wherein the cancer is a prostate cancer.

54. The method according to any one of claims 49 to 52, wherein the cancer is a glioblastoma.

55. The method according to any one of claims 49 to 52, wherein the cancer is of hematological origin.

56. The method according to any one of claims 49 to 55, wherein the subject is a human.

57. The method according to any one of claims 49 to 56, wherein the level of hydroxylation of ACC2 at proline 450 by the cancer cells is less than or equal to 90% of normal cells of the same histological type from which the cancer cells are derived.

58. The method according to any one of claims 49 to 56, wherein the level of hydroxylation of ACC2 at proline 450 by the cancer cells is less than or equal to 80% of normal cells of the same histological type from which the cancer cells are derived.

59. The method according to any one of claims 49 to 56, wherein the level of hydroxylation of ACC2 at proline 450 by the cancer cells is less than or equal to 70% of normal cells of the same histological type from which the cancer cells are derived.

60. The method according to any one of claims 49 to 56, wherein the level of hydroxylation of ACC2 at proline 450 by the cancer cells is less than or equal to 50% of normal cells of the same histological type from which the cancer cells are derived.

61. The method according to any one of claims 49 to 56, wherein the level of hydroxylation of ACC2 at proline 450 by the cancer cells is less than or equal to 25% of normal cells of the same histological type from which the cancer cells are derived.

62. The method according to any one of claims 49 to 56, wherein the level of hydroxylation of ACC2 at proline 450 by the cancer cells is less than or equal to 15% of normal cells of the same histological type from which the cancer cells are derived.

63. A method for treating a subject having a cancer comprising cancer cells with an elevated level of hydroxylation of ACC2 at proline 450 relative to SEQ ID NO:2, the method comprising administering to the subject a glycolytic pathway inhibitor in an amount effective to treat the cancer.

64. A method for treating a subject having a cancer, the method comprising administering to the subject a glycolytic pathway inhibitor in an amount effective to treat the cancer, wherein the cancer has been identified as comprising cancer cells with an elevated level of hydroxylation of ACC2 at proline 450 relative to SEQ ID NO:2.

65. A method for treating a subject having a cancer, the method comprising:
receiving the results of a test determining that the subject's cancer comprises cancer cells with an elevated level of hydroxylation of ACC2 at proline 450 relative to SEQ ID NO:2; and
ordering administration of an effective amount of a glycolytic pathway inhibitor to the subject.

66. A method for treating a subject having a cancer, the method comprising:
requesting a test, or the results of a test, determining that the subject's cancer comprises cancer cells with an elevated level of hydroxylation of ACC2 at proline 450 relative to SEQ ID NO:2; and
ordering administration of an effective amount of a glycolytic pathway inhibitor to the subject.

67. The method according to any one of claims 63 to 66, wherein the cancer is a pancreatic cancer.

68. The method according to any one of claims 63 to 66, wherein the cancer is a kidney cancer or bladder cancer.

69. The method according to any one of claims 63 to 66, wherein the cancer is a melanoma, a lung cancer, a follicular lymphoma, a breast cancer, a colorectal cancer, or an ovarian cancer.

70. The method according to any one of claims 63 to 66, wherein the subject is a human.

71. The method according to any one of claims 63 to 70, wherein the level of hydroxylation of ACC2 at proline 450 by the cancer cells is at least 20% greater than that of normal cells of the same histological type from which the cancer cells are derived.

72. The method according to any one of claims 63 to 70, wherein the level of hydroxylation of ACC2 at proline 450 by the cancer cells is at least 50% greater than that of normal cells of the same histological type from which the cancer cells are derived.

73. The method according to any one of claims 63 to 70, wherein the level of hydroxylation of ACC2 at proline 450 by the cancer cells is at least 75% greater than that of normal cells of the same histological type from which the cancer cells are derived.

74. The method according to any one of claims 63 to 70, wherein the level of hydroxylation of ACC2 at proline 450 by the cancer cells is at least 100% greater than that of normal cells of the same histological type from which the cancer cells are derived.

75. The method according to any one of claims 63 to 70, wherein the level of hydroxylation of ACC2 at proline 450 by the cancer cells is at least 2.5 fold greater than that of normal cells of the same histological type from which the cancer cells are derived.

76. The method according to any one of claims 63 to 70, wherein the level of hydroxylation of ACC2 at proline 450 by the cancer cells is at least 5 fold greater than that of normal cells of the same histological type from which the cancer cells are derived.

77. An isolated antibody, or fragment thereof, that preferentially binds to an ACC2 polypeptide when hydroxylated at proline 450 relative to SEQ ID NO:2 over the ACC2 polypeptide when not hydroxylated at proline 450 relative to SEQ ID NO:2.

78. The isolated antibody, or fragment thereof, according to claim 77, wherein the isolated antibody, or fragment thereof, only binds to an ACC2 polypeptide when hydroxylated at proline 450 relative to SEQ ID NO:2.

79. An isolated antibody, or fragment thereof, that only binds to an ACC2 polypeptide when not hydroxylated at proline 450 relative to SEQ ID NO:2.

80. An isolated antibody, or fragment thereof, that specifically binds to an ACC2 polypeptide that is hydroxylated at proline 450 relative to SEQ ID NO:2, wherein the antibody specifically binds to an epitope that is within the amino acid sequence of any one of SEQ ID NOs:6-9.

81. The isolated antibody, or fragment thereof, of any one of claims 77 to 80, wherein the fragment is a Fab, Fv, single-chain (scFv), Fab′, or F(ab′)₂.

82. The isolated antibody, or fragment thereof, according to any one of claims 77 to 80, wherein the antibody is a minibody or domain antibody.

83. The isolated antibody, or fragment thereof, of any one of claims 77 to 80, wherein the antibody is a whole antibody.

84. A method for detecting P450-hydroxylated ACC2 in a biological sample, the method comprising:
(a) contacting a biological sample with at least one antibody according to any one of claims 77 to 83 under conditions suitable for formation of a complex between the antibody and ACC2 that is hydroxylated at proline 450 relative to SEQ ID NO:2, if such hydroxylated ACC2 is present in the biological sample; and
(b) detecting the presence of the complex in the biological sample, wherein the presence of the complex indicates the presence of hydroxylated ACC2 in the biological sample.

85. A method for detecting P450-hydroxylated ACC2 in a biological sample, the method comprising:
(a) contacting a biological sample with at least one antibody according to any one of claims 77 to 83 under conditions suitable for formation of a complex between the antibody and ACC2 that is hydroxylated at proline 450 relative to SEQ ID NO:2, if such hydroxylated ACC2 is present in the biological sample;
(b) contacting the complex of (a) with a detection reagent; and
(c) detecting the presence or amount of the detection reagent as a measure of the presence or amount of the complex in the biological sample, wherein the presence of the complex indicates the presence of P450-hydroxylated ACC2 in the biological sample.

86. A method for detecting P450-hydroxylated ACC2 in a biological sample, the method comprising:
(a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample; and
(b) detecting the presence or amount of the detection reagent as a measure of the presence or amount of the complex in the biological sample, wherein the presence of the complex indicates the presence of hydroxylated ACC2 in the biological sample.

87. A kit for the detection of P450-hydroxylated ACC2 in a biological sample, the kit comprising: (a) at least one antibody according to any one of claims 77 to 83, and (b) at least one secondary reagent.

88. The kit according to claim 87, wherein the at least one secondary reagent is an antibody that binds to the at least one antibody of (a).

89. A nucleic acid encoding the antibody according to any one of claims 77 to 83.

90. An expression vector comprising the nucleic acid of claim 89.

91. A cell comprising the nucleic acid according to claim 89 or the expression vector according to claim 90.

92. An isolated polypeptide comprising at least 10 consecutive amino acids of SEQ ID NO:2, but no more than 2000 consecutive amino acids of SEQ ID NO:2, wherein the polypeptide comprises proline 450 of SEQ ID NO:2.

93. An isolated polypeptide comprising at least 10 consecutive amino acids of SEQ ID NO:2, including proline 450 of SEQ ID NO:2, wherein the polypeptide comprises at most 98% of SEQ ID NO:2.

94. An isolated polypeptide comprising at least 10 consecutive amino acids of SEQ ID NO:2 inclusive of the proline residue at position 450 of SEQ ID NO:2, wherein the proline residue at position 450 is mutated, modified, or deleted.

95. A polypeptide comprising: (i) the amino acid sequence depicted in SEQ ID NO:2, wherein the proline residue at position 450 is mutated, modified, or deleted; (ii) a variant of the amino acid sequence depicted in SEQ ID NO:2 having not more than 100 amino acid substitutions, deletions, or insertions, and wherein the proline residue at position 450 is mutated, modified, or deleted; or (iii) an amino acid sequence that is at least 80% identical to any one of the amino acid sequences depicted in SEQ ID NO:2, wherein the proline residue at position 450 is mutated, modified, or deleted.

96. The isolated polypeptide according to claim 94 or 95, wherein the proline residue at position 450 is hydroxylated.

97. A nucleic acid encoding the polypeptide according to any one of claims 92 to 95.

98. An expression vector comprising the nucleic acid of claim 97.

99. A cell comprising the nucleic acid according to claim 97 or the expression vector according to claim 98.

100. A method for determining whether a cancer is susceptible to a fatty acid oxidation inhibitor, the method comprising:
(a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and
(b) detecting the presence or amount of the detection reagent as a measure of the presence or amount of the complex in the biological sample, wherein a reduced level of ACC2 hydroxylated at proline 450, relative to a control level, indicates that the cancer is susceptible to a fatty acid oxidation inhibitor.

101. A method for determining whether a cancer patient will benefit from treatment with a fatty acid oxidation inhibitor, the method comprising:
(a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and
(b) detecting the presence or amount of the detection reagent as a measure of the presence or amount of the complex in the biological sample, wherein a reduced level of ACC2 hydroxylated at proline 450, relative to a control level, indicates that the cancer patient will benefit from treatment with a fatty acid oxidation inhibitor.

102. A method for determining whether a cancer is susceptible to a glycolytic pathway inhibitor, the method comprising:
(a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and
(b) detecting the presence or amount of the detection reagent as a measure of the presence or amount of the complex in the biological sample, wherein an elevated level of ACC2 hydroxylated at proline 450, relative to a control level, indicates that the cancer is susceptible to a glycolytic pathway inhibitor.

103. A method for determining whether a cancer patient will benefit from treatment with a glycolytic pathway inhibitor, the method comprising:
(a) contacting a biological sample with a detection reagent under conditions suitable for formation of a complex between the detection reagent and ACC2 that is hydroxylated at proline 450 relative to SEQ ID NO:2, if such hydroxylated ACC2 is present in the biological sample, wherein the biological sample comprises cancer cells or lysates of cancer cells from a subject; and
(b) detecting the presence or amount of the detection reagent as a measure of the presence or amount of the complex in the biological sample, wherein an elevated level of ACC2 hydroxylated at proline 450, relative to a control level, indicates that the cancer patient will benefit from treatment with a glycolytic pathway inhibitor.

104. A method for increasing fatty acid oxidation by a cell, the method comprising contacting the cell with a compound that inhibits the hydroxylation of ACC2 at proline 450 relative to SEQ ID NO:2 by PHD3 in an amount effective to increase fatty acid oxidation by the cell.

105. A method for increasing fatty acid oxidation in a subject in need thereof, the method comprising administering to the subject a compound that inhibits the hydroxylation of ACC2 at proline 450 relative to SEQ ID NO:2 by PHD3 in an amount effective to increase fatty acid oxidation in the subject.

106. A method for promoting weight loss in a subject, the method comprising administering to the subject a compound that inhibits the hydroxylation of ACC2 at proline 450 relative to SEQ ID NO:2 by PHD3 in an amount effective to promote weight loss in the subject.

107. A method for treating cardiovascular disease in a subject, the method comprising administering to the subject a compound that inhibits the hydroxylation of ACC2 at proline 450 relative to SEQ ID NO:2 by PHD3 in an amount effective to treat the cardiovascular disease in the subject.

108. A method for treating a subject afflicted with a metabolic syndrome, diabetes, obesity, atherosclerosis, or cardiovascular disease, the method comprising administering to the subject a compound that inhibits the hydroxylation of ACC2 at proline 450 relative to SEQ ID NO:2 by PHD3 in an amount effective to treat the metabolic syndrome, diabetes, obesity, atherosclerosis, or cardiovascular disease.

109. The method according to any one of claims 105 to 108, wherein the subject is obese.

110. The method according to any one of claims 105 to 108, wherein the subject is overweight.

111. The method according to any one of claims 105 to 110, wherein the subject has coronary artery disease.

112. The method according to any one of claims 105 to 111, wherein the subject has diabetes.

113. A method for treating or delaying the onset of an obesity-related disorder in a subject, the method comprising administering to the subject a compound that inhibits the hydroxylation of ACC2 at proline 450 relative to SEQ ID NO:2 by PHD3 in an amount effective to treat or delay the onset of an obesity-related disorder in the subject.

114. A method for treating a subject having a cancer, the method comprising:
administering to the subject an inhibitor of PHD3 to thereby sensitize the cancer to a fatty acid oxidation (FAO) inhibitor; and
administering to the subject an effective amount of a FAO inhibitor to treat the cancer, wherein the effective amount of the FAO inhibitor is lower than the amount effective to treat the cancer in the absence of PHD3 inhibition.

115. The method according to claim 114, wherein the inhibitor of PHD3 is administered first in time and the FAO inhibitor administered second in time.

116. The method according to claim 114, wherein the inhibitor of PHD3 and the FAO inhibitor are administered concurrently.

117. The method according to any one of claims 114 to 116, wherein the inhibitor of PHD3 binds to and inhibits the activity of PHD3.

118. The method according to claim 117, wherein the inhibitor of PHD3 is a small molecule, a macrocycle compound, a polypeptide, a nucleic acid, or a nucleic acid analog.

119. The method according to any one of claims 114 to 116, wherein the inhibitor of PHD3 reduces the expression or stability of an mRNA encoding PHD3 protein.

120. The method according to claim 119, wherein the compound is an antisense oligonucleotide, an siRNA, an shRNA, or a ribozyme.

121. The method according to any one of claims 114 to 120, wherein the cancer is a prostate cancer, a glioblastoma, or a cancer is of hematological origin.

122. The method according to any one of claims 114 to 121, wherein the subject is a human.

123. The method according to any one of claims 114 to 122, wherein PHD3 expression by the cancer cells is less than or equal to 90% of normal cells of the same histological type from which the cancer cells are derived.

124. The method according to any one of claims 114 to 123, further comprising determining whether the cancer cells have reduced PHD3 expression.

125. A method for identifying a modulator of PHD3 activity, the method comprising:
contacting, in the presence of a substrate ACC2 protein, a PHD3 protein or an enzymatically-active fragment thereof with a candidate compound; and
detecting hydroxylation of the substrate ACC2 protein by the PHD3 protein or enzymatically-active fragment thereof, wherein a difference in the amount of hydroxylation of the substrate ACC2 protein by the PHD3 protein or enzymatically-active fragment thereof in the presence of the candidate compound, as compared to the amount of hydroxylation of the substrate ACC2 protein by the PHD3 protein or enzymatically-active fragment thereof in the absence of the candidate compound, indicates that the candidate compound modulates PHD3 activity.

126. A method of screening for candidate compounds which are capable of modulating the activity of a PHD3 protein or enzymatically-active fragment thereof to hydroxylate a substrate ACC2 protein, the method comprising determining whether at least one candidate compound has the property of modulating the activity of a PHD3 protein or enzymatically-active fragment thereof to hydroxylate a substrate ACC2 protein under conditions in which the PHD3 protein or enzymatically-active fragment thereof is capable of hydroxylating the substrate ACC2 protein in the absence of the candidate compound.

127. The method according to claim 126, wherein the method comprises:
(a) contacting at least one candidate compound, a substrate ACC2 protein and the PHD3 protein or enzymatically-active fragment thereof under conditions in which the PHD3 protein or enzymatically-active fragment thereof is capable of hydroxylating position P450 of the substrate ACC2 protein in the absence of the candidate compound;
(b) determining whether the candidate compound modulates the hydroxylation of the substrate ACC2 protein at position P450 by the PHD3 protein or enzymatically-active fragment thereof; and
(c) identifying the candidate compound as a modulator of PHD3 protein if the compound modulates the hydroxylation of the substrate ACC2 protein at position P450 by the PHD3 protein or enzymatically-active fragment thereof.

128. The method according to any one of claims 125 to 127, wherein the candidate compound inhibits hydroxylation of the substrate ACC2 protein by the PHD3 protein or enzymatically-active fragment thereof.

129. The method according to any one of claims 125 to 128, wherein the contacting occurs in a cell.

130. The method according to claim 129, wherein the cell comprises one or both of: (a) a transgene encoding the substrate ACC2 protein and (b) a transgene encoding the PHD3 protein or enzymatically-active fragment thereof.

131. The method according to any one of claims 125 to 127, wherein the contacting occurs in vitro.

132. A method of identifying an agent which inhibits hydroxylation of a substrate ACC2 protein by a PHD3 protein or enzymatically-active fragment thereof, the method comprising:
introducing into a cell that expresses a substrate ACC2 protein a vector that expresses a PHD3 protein or enzymatically-active fragment thereof;
contacting the cell with a test compound under conditions in which P450 in the substrate ACC2 protein is hydroxylated by PHD3 in the absence of the test substance; and
determining hydroxylation of the substrate, wherein a decrease in the hydroxylation of P450 of the substrate ACC2 protein in the presence of the test compound as compared to the hydroxylation of P450 of the substrate ACC2 protein in the absence of the test compound identifies the test substance as an agent that inhibits hydroxylation of ACC2 by PHD3.
